(19) United States
(12) Patent Application Publication
(10) Pub. No.: US 2024/0383850 A1
(43) Pub. Date: Nov. 21, 2024

(54) INDOLE DERIVATIVES AS SEROTONERGIC AGENTS USEFUL FOR THE TREATMENT OF DISORDERS RELATED THERETO

(71) Applicant: Mindset Pharma Inc., St-Laurent (CA)

(72) Inventors: Abdelmalik Slassi, Mississauga (CA); Joseph A. Araujo, Grimsby (CA); Guy Higgins, Elora (CA)

(73) Assignee: Mindset Pharma Inc., St-Laurent (CA)

(21) Appl. No.: 18/683,842

(22) PCT Filed: Aug. 19, 2022
(86) PCT No.: PCT/CA2022/051264

§ 371 Date: Feb. 15, 2024

Related U.S. Application Data
(60) Provisional application No. 63/260,470, filed on Aug. 20, 2021.
(60) Provisional application No. 63/326,406, filed on Apr. 1, 2022.
(60) Provisional application No. 63/347,845, filed on Jun. 1, 2022.

(51) Int. Cl. C07D 209/18; A61K 31/404; A61K 31/454; A61K 45/06; C07D 401/04; C07D 403/06

(52) U.S. Cl. CPC C07D 209/18; A61K 31/404; A61K 31/454; A61K 45/06; C07D 401/04; C07D 403/06

(57) ABSTRACT

The present application relates to indole compounds of general formula I, to processes for their preparation, to compositions comprising them and to their use in activation of a serotonin receptors in a cell, as well as to treating diseases, disorders or conditions by activation of a serotonin receptors in a cell. The diseases, disorders or conditions include, for example, psychosis, mental illnesses and CNS disorders, Formula I (I) wherein Q is selected from (Q1), (Q2), (Q2′), (Q3), (Q4) and (Q5).

### CROSS-REFERENCE TO RELATED APPLICATIONS

The present application claims the benefit of priority to co-pending U.S. provisional patent application Ser. No. 63/260,470 filed on Aug. 20, 2021, to co-pending U.S. provisional patent application Ser. No. 63/326,406 filed on Apr. 1 2022 and to co-pending U.S. provisional patent application Ser. No. 63/347,845 filed on Jun. 1 2022, the contents of each of which are incorporated herein by reference in their entirety.

### FIELD

The application relates to indole compounds for the treatment of different conditions that are treated by activation of serotonin receptors, for example, mental illnesses and neurological disease, in the fields of psychiatry, neurobiology and pharmacotherapy.

### BACKGROUND OF THE APPLICATION

The present applicant has several co-pending applications disclosing novel indole derivatives as serotonergic psychedelic agents for the treatment of various CNS disorders, including PCT patent application publication nos. WO2021/155470, WO2021/155468 and WO2021/155467, and PCT application S.N. PCT/CA2022/-50295 and U.S. provisional application Ser. No. 63/260,470 These applications describe the various pharmaceutical applications for such agents, including in the treatment of mental illnesses and neurological disease, and use in the fields of psychiatry, neurobiology and pharmacotherapy.

Serotonin syndrome (SS), also referred to as serotonin toxicity, is a potentially life-threatening drug-induced condition associated with increased serotonergic activity in both the peripheral and central nervous systems. It is characterized by a dose-relevant spectrum of clinical findings related to the level of free serotonin (5-hydroxytrypatmine or 5-HT) or 5-HT receptor activation. The predominant 5-HT receptors involved in SS are the 5-HT_{1A} and 5-HT_{2A} subtypes. Some of the main clinical symptoms include neuromuscular abnormalities, autonomic hyperactivity and mental state changes. Severe SS is usually caused by the simultaneous introduction of 2 or more serotonergic drugs, but the syndrome can also occur after the introduction of only one serotonergic drug in susceptible individuals, the addition of one or more therapeutics to a subject on long-term serotonergic drug therapy (such as a monoamine oxidase inhibitor), or after an overdose. SS has been observed across all age groups and is increasing in incidence, likely due to the increasing therapeutic use of serotonergic drugs. Several review articles have been published recently on this syndrome highlighting its significance in current healthcare (see for example: Francescangeli, J. et al., Int. J. Mol. Sci. 2019, 20:2288; Birmes, P. et al., Can. Med. Assoc. J. 2003, 168(11), 1439-1442; Koleva, K. and Nikolov, R., Pharmacia, 2018, 65(1), 64-70; Scotten, W. J. et al., Int. J. Tryptophan Res. 2019, 12, 1-14).

Conjugation of active pharmaceutical agents with fatty acids has been used, for example, to increase half-life, to enhance cellular uptake and retention, for targeted tumor delivery, to reduce chemoresistance in cancer and to improve blood brain barrier (BBB) penetration (Bhat, M. et al., Chem. Phys. Lipids, 2021, 236, 105053). This approach has been applied to many classes of drugs, including for example, non-steroid anti-inflammatory drugs (NSAIDs), angiotensin-converting enzyme inhibitors, angiotensin, nucleosides and testosterone.

It has been reported that fatty acids may enhance the activity of certain antidepressant drugs at low non-antidepressant doses (Laino, C. H. et al., Europ. J. Pharmacol. (2010), 648:117-126; Carlezon, W. A. Jr. et al. Biol. Psychiatry (2005), 57:343-350). Some reports show that omega-3 fatty acids have antidepressant activity when administered on a chronic basis (Lakhwani, L. et al., Acta Poloniae Pharmaceutica—Drug Res. (2007), 64:271-276). Docosahexanaenoic acid (DHA) has been reported to provide a synergistic effect in enhancing the absorption of carotenoids, such as lutein (US patent application no. US2006/0270739). Moreover, DHA has been implicated in augmented brain and cognitive development and maintenance in aging and neuropsychiatric disorders in both humans and animals (Ciappolino, V. et al., Nutrients (2019), 11:769 doi 10.3990/nu11040769; Lauritzen, L. et al., Nutrients (2016), 8:6 doi 10.3390/nu8010006; Weiser, M. J. et al., Nutrients (2016), 8:99 doi 10.3990/nu8020099).

### SUMMARY OF THE APPLICATION

Compounds of the present application modulate the activity of serotonin receptor subtypes, in particular 5-HT2A, by direct binding to these receptors and/or via the corresponding N-unsubstituted analogs which are produced in vivo via hydrolysis. Accordingly, in some embodiments, compounds of the present application act as prodrugs.

Accordingly, the present application includes compounds of Formula I:

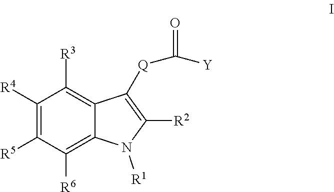

or a pharmaceutically acceptable salt, solvate and/or prodrug thereof,
wherein:
R¹ is selected from H and C₁₋₆alkyl;
Q is selected from Q1, Q2, Q2′, Q3, Q4 and Q5:

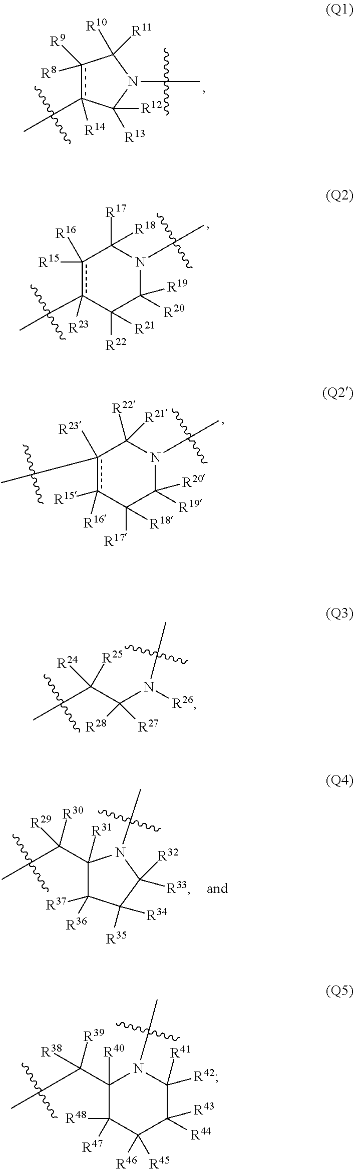

[symbol] is a single bond or a double bond provided, when [symbol] in Q1 is a double bond then R⁸ and R¹⁴ are not present, when [symbol] in Q2 is a double bond then R¹⁶ and R²³ are not present and when [symbol] in Q2′ is a double bond then R^{16′} and R^{23′} are not present;
[symbol] over a bond means that the bond is attached to a remaining portion of the compound, wherein the [symbol] over the bond attached to the N in Q1, Q2, Q2′, Q3, Q4 and Q5 indicates the bond that is attached to C(O)—Y and the [symbol] over the bond attached to the C in Q1, Q2, Q2′, Q3, Q4 and Q5 indicates the bond that is attached to the indole;
Y is selected from R⁴⁹, O—R⁴⁹ and O—C₁₋₄alkylene-O—C(O)—R⁴⁹;
R⁴⁹ is selected from C₇₋₃₀alkyl and C₇₋₃₀alkenyl;
R² is selected from H, halo, C₁₋₆alkyl and C₁₋₆alkoxy;
one of R³ and R⁴ is selected from H, OH, halo, C₁₋₆alkyl and C₁₋₆alkoxy and the other or R³ and R⁴ is selected from H, OH, halo, C₁₋₆alkyl, C₁₋₆alkoxy and OP(O)(OR⁵⁰)₂;
R⁵ and R⁶ are independently selected from H, OH, halo, C₁₋₆alkyl and C₁₋₆alkoxy; R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R^{15′}, R^{16′}, R^{17′}, R^{18′}, R^{19′}, R²⁰, R^{21′}, R²², R^{23′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, halo and C₁₋₆alkyl; R²⁶ is selected from H and C₁₋₆alkyl;
R⁵⁰ is selected from H and C₁₋₆alkyl; and
all available hydrogen atoms are optionally and independently replaced with a fluorine and/or chlorine atom and/or all available atoms are optionally replaced with alternate isotope thereof,
provided that
   (1) when Q is Q3, either (i) R²⁶ is C₁₋₆alkyl or (ii) Y is selected from O—R⁴⁹ and O—C₁₋₄alkylene-O—C(O)—R⁴⁹, or (iii) the compound of Formula I comprises at least one deuterium, and
   (2) when Q is Q2, [symbol] is a single bond and R¹⁵, R¹⁶, R¹⁷, R¹⁸, R¹⁹, R²⁰, R²¹, R²² and R²³ are all H, then either (i) Y is selected from O—R⁴⁹ and O—C₁₋₄alkylene-O—C(O)—R⁴⁹, or (ii) the compound of Formula I comprises at least one deuterium.

The present application also includes a pharmaceutical composition comprising one or more compounds of the application and pharmaceutically acceptable carrier.

Also included in the present application is a method of treating a disease, disorder or condition by activation of a serotonin receptor comprising administering a therapeutically effective amount of one or more compounds of the application to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

The present application will be described in greater detail with reference to the attached drawings in which:

FIG. 1 shows the scores for exemplary compound 1-51 in the mouse head twitch assays at doses of up to 60 mg/kg.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I and includes pharmaceutically acceptable salts, solvates and/or prodrugs thereof as well as all stereoisomers and regioisomers.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used in the present application, the singular forms “a”, “an” and “the” include plural references unless the content clearly dictates otherwise. For example, an embodiment including “a compound” should be understood to present certain embodiments with one compound, or two or more additional compounds.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers and/or steps and also exclude the presence of other unstated features, elements, components, groups, integers and/or steps.

The term “consisting essentially of”, as used herein, is intended to specify the presence of the stated features, elements, components, groups, integers and/or steps as well as those that do not materially affect the basic and novel characteristic(s) of these features, elements, components, groups, integers and/or steps.

In embodiments comprising an “additional” or “second” component, such as an additional or second compound, the second component as used herein is chemically different from the other components or first component. A “third” component is different from the other, first and second components and further enumerated or “additional” components are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “solvate” as used herein means a compound, or a salt and/or prodrug of a compound, wherein molecules of a suitable solvent are incorporated in the crystal lattice.

The term “prodrug” as used herein means a compound, or salt of a compound, that, after administration, is converted into an active drug.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or 6 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₁₀alkylene means an alkylene group having 1, 2, 3, 4, 5, 6, 7, 8, 9 or 10 carbon atoms. All alkyl groups are optionally fluorosubstituted unless otherwise indicated.

As used herein, the term “alkoxy” as used herein, alone or in combination, includes an alkyl group connected to an oxygen-connecting atom.

The term “cycloalkyl,” as used herein, whether it is used alone or as part of another group, means a saturated carbocyclic group containing from 3 to 6 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5 or 6 carbon atoms.

The term “heterocycloalkyl” as used herein, whether it is used alone or as part of another group, refers to cyclic groups containing at least one non-aromatic ring containing from 3 to 6 atoms in which one or more of the atoms are a heteromoiety selected from O, S, S(O), SO₂, N, NH and NHC₁₋₆alkyl and the remaining atoms are C. Heterocycloalkyl groups are either saturated or unsaturated (i.e. contain one or more double bonds). When a heterocycloalkyl group contains the prefix Cₙ₁₋ₙ₂ or “n1 to n2” this prefix indicates the number of carbon atoms in the corresponding carbocyclic group, in which one or more, suitably 1 to 4, of the ring atoms is replaced with a heteromoeity as selected from O, S, S(O), SO₂, N, NH and NHC₁₋₆alkyl and the remaining atoms are C.

The term “heteroaryl” as used herein, whether it is used alone or as part of another group, refers to cyclic groups containing at least one heteroaromatic ring containing 5-6 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 4, of the ring atoms is replaced with a heteroatom as defined above.

The term “halogen” (or “halo”) whether it is used alone or as part of another group, refers to a halogen atom and includes fluoro, chloro, bromo and iodo.

The term “haloalkyl” as used herein refers to an alkyl group as defined above in which one or more of the available hydrogen atoms have been replaced with a halogen. Thus, for example, “C₁₋₆haloalkyl” refers to a C₁ to C₆ linear or branched alkyl group as defined above with one or more halogen substituents.

The term “deuteroalkyl” as used herein refers to an alkyl group as defined above in which one or more of the available hydrogen atoms have been replaced with a deuterium. Thus, for example, “C₁₋₆deuteroalkyl” refers to a C₁ to C₆ linear or branched alkyl group as defined above with one or more dueterium substituents.

The term “available”, as in “available hydrogen atoms” or “available atoms” refers to atoms that would be known to a person skilled in the art to be capable of replacement by a substituent.

As used herein, the term “one or more” item includes a single item selected from the list as well as mixtures of two or more items selected from the list.

The term “alternate isotope thereof” as used herein refers to an isotope of an element that is other than the isotope that is most abundant in nature.

In the compounds of general Formula I and pharmaceutically acceptable salts and/or solvates thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general Formula I and pharmaceutically acceptable salts and/or solvates thereof. For example, different isotopic forms of hydrogen (H) include protium (¹H), deuterium (²H) and tritium (³H). Protium is the predominant hydrogen isotope found in nature.

The term “compound” refers to the compound and, in certain embodiments, to the extent they are stable, any hydrate or solvate thereof. A hydrate is the compound complexed with water and a solvate is the compound complexed with a solvent, which may be an organic solvent or an inorganic solvent. A “stable” compound is a compound that can be prepared and isolated and whose structure and properties remain or can be caused to remain essentially unchanged for a period of time sufficient to allow use of the compound for the purposes described herein (e.g., therapeutic administration to a subject). The compounds of the present application are limited to stable compounds embraced by general Formula (I-A), or pharmaceutically acceptable salts and/or solvates thereof.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus, the methods of the present application are applicable to both human therapy and veterinary applications.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early neurological disease can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alliteratively comprise a series of administrations.

As used herein, the term “effective amount” or “therapeutically effective amount” means an amount of one or more compounds of the application that is effective, at dosages and for periods of time necessary to achieve the desired result. For example, in the context of treating a disease, disorder or condition mediated or treated by agonism or activation of serotonergic receptors and downstream second messengers, an effective amount is an amount that, for example, increases said activation compared to the activation without administration of the one or more compounds.

“Palliating” a disease, disorder or condition means that the extent and/or undesirable clinical manifestations of a disease, disorder or condition are lessened and/or time course of the progression is slowed or lengthened, as compared to not treating the disorder.

The term “administered” as used herein means administration of a therapeutically effective amount of one or more compounds or compositions of the application to a cell, tissue, organ or subject.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition treated or treatable by activation a serotonin receptor, for example 5-HT_{2A} and particularly using a serotonin receptor agonist, such as one or more compounds of the application herein described. The disease, disorder or condition may also be treated or treatable via alternative mechanisms, for example by modulation, deactivation, antagonism or reverse agonism of a serotonin receptor, including 5-HT_{2A} and/or 5-HT_{1A}.

The term “treating a disease, disorder or condition by activation of a serotonin receptor” as used herein means that the disease, disorder or condition to be treated is affected by, modulated by and/or has some biological basis, either direct or indirect, that includes serotonergic activity, in particular increases in serotonergic activity. These diseases respond favourably when serotonergic activity associated with the disease, disorder or condition is agonized by one or more of the compounds or compositions of the application.

The term “activation” as used herein includes agonism, partial agonist and positive allosteric modulation of a serotonin receptor.

The terms “5-HT_{1A}” and “5-HT_{2A}” are used herein mean the 5-HT_{1A} and 5-HT_{2A} receptor subtypes of the 5-HT2 serotonin receptor.

The term “therapeutic agent” as used herein refers to any drug or active agent that has a pharmacological effect when administered to a subject.

### II. Compounds

The present application includes compounds of Formula I:

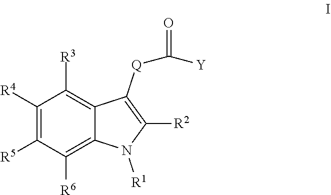

or a pharmaceutically acceptable salt, solvate and/or prodrug thereof,
wherein:
R¹ is selected from H and C₁₋₆alkyl;
Q is selected from Q1, Q2, Q2′, Q3, Q4 and Q5:

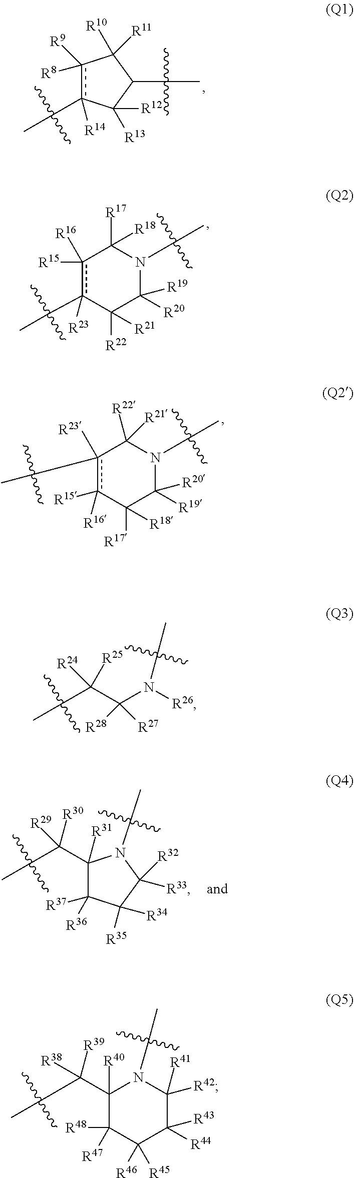

[symbol] is a single bond or a double bond provided that when [symbol] in Q1 is a double bond then R⁸ and R¹⁴ are not present, when [symbol] in Q2 is a double bond then R¹⁶ and R²³ are not present and when [symbol] in Q2′ is a double bond then R^{17′} and R^{25′} are not present;
[symbol] over a bond means that the bond is attached to a remaining portion of the compound, wherein the [symbol] over the bond attached to the N in Q1, Q2, Q2′, Q3, Q4 and Q5 indicates the bond that is attached to C(O)—Y and the [symbol] over the bond attached to the C in Q1, Q2, Q2′, Q3, Q4 and Q5 indicates the bond that is attached to the indole;
Y is selected from R⁴⁹, O—R⁴⁹ and O—C₁₋₄alkylene-O—C(O)—R⁴⁹;
R⁴⁹ is selected from C₇₋₃₀alkyl and C₇₋₃₀alkenyl;
R² is selected from H, halo, C₁₋₆alkyl and C₁₋₆alkoxy;
one of R³ and R⁴ is selected from H, OH, halo, C₁₋₆alkyl and C₁₋₆alkoxy and the other of R³ and R⁴ is selected from H, OH, halo, C₁₋₆alkyl, C₁₋₆alkoxy and OP(O)(OR⁵⁰)₂;
R⁵ and R⁶ are independently selected from H, OH, halo, C₁₋₆alkyl and C₁₋₆alkoxy;
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R^{15′}, R^{16′}, R^{17′}, R^{18′}, R^{19′}, R^{20′}, R^{21′}, R^{22′}, R^{23′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, halo and C₁₋₆alkyl;
R²⁶ is selected from H and C₁₋₆alkyl; and
all available hydrogen atoms are optionally and independently replaced with a fluorine and/or chlorine and/or all available atoms are optionally replaced with alternate isotope thereof,
provided that
   (1) when Q is Q3, either (i) R²⁶ is C₁₋₆alkyl or (ii) Y is selected from O—R⁴⁹ and O—C₁₋₄alkylene-O—C(O)—R⁴⁹, or (iii) the compound of Formula I comprises at least one deuterium, and
   (2) when Q is Q2, [symbol] is a single bond and R¹⁵, R¹⁶, R¹⁷, R¹⁸, R¹⁹, R²⁰, R²¹, R²² and R²³ are all H, then either (i) Y is selected from O—R⁴⁹ and O—C₁₋₄alkylene-O—C(O)—R⁴⁹, or (ii) the compound of Formula I comprises at least one deuterium.

The present application also includes compounds of Formula I:

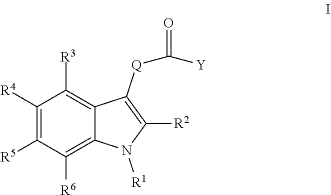

or a pharmaceutically acceptable salt, solvate and/or prodrug thereof,
wherein:
R¹ is selected from H and C₁₋₆alkyl;
Q is selected from Q1, Q2, Q3, Q4 and Q5:

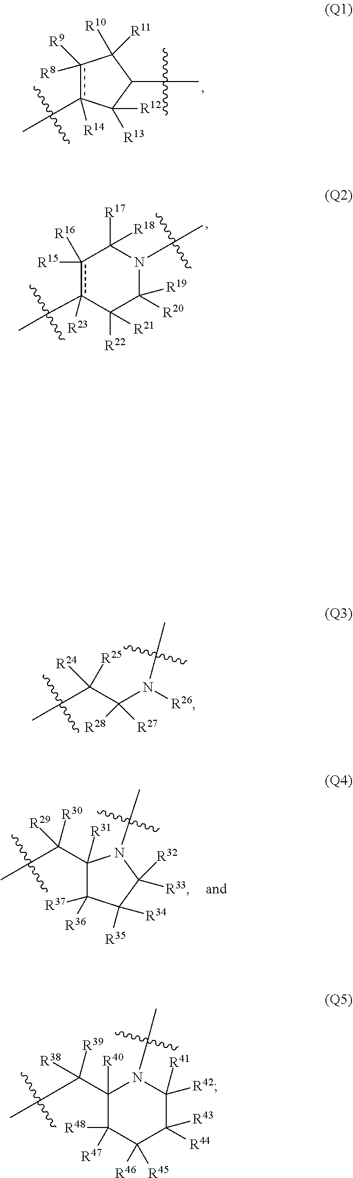

[symbol] is a single bond or a double bond provided that when [symbol] in Q1 is a double bond then R⁸ and R¹⁴ are not present and when [symbol] in Q2 is a double bond then R¹⁶ and R²³ are not present;
[symbol] over a bond means that the bond is attached to a remaining portion of the compound, wherein the [symbol] over the bond attached to the N in Q1, Q2, Q2′, Q3, Q4 and Q5 indicates the bond that is attached to C(O)—Y and the other [symbol] over the bond attached to the C in Q1, Q2, Q2′, Q3, Q4 and Q5 indicates the bond that is attached to the indole;
Y is selected from R⁴⁹, O—R⁴⁹ and O—C₁₋₄alkylene-O—C(O)—R⁴⁹;
R⁴⁹ is selected from C₇₋₃₀alkyl and C₇₋₃₀alkenyl;
R² is selected from H, halo, C₁₋₆alkyl and C₁₋₆alkoxy;
R³, R⁴, R⁵ and R⁶ are independently selected from H, OH, halo, C₁₋₆alkyl and C₁₋₆alkoxy;
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R^{15′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, halo and C₁₋₆alkyl;
R²⁶ is selected from H and C₁₋₆alkyl;
R⁵⁰ is selected from H and C₁₋₆alkyl; and
all available hydrogen atoms are optionally and independently replaced with a fluorine atom and/or all available atoms are optionally replaced with alternate isotope thereof,
provided that
   (1) when Q is Q3, either (i) R²⁶ is C₁₋₆alkyl or (ii) Y is selected from O—R⁴⁹ and O—C₁₋₄alkylene-O—C(O)—R⁴⁹, or (iii) the compound of Formula I comprises at least one deuterium, and
   (2) when Q is Q2, [symbol] is a single bond and R¹⁵, R¹⁶, R¹⁷, R¹⁸, R¹⁹, R²⁰, R²¹, R²² and R²³ are all H, then either (i) Y is selected from O—R⁴⁹ and O—C₁₋₄alkylene-O—C(O)—R⁴⁹, or (ii) the compound of Formula I comprises at least one deuterium.

In some embodiments, all available hydrogen atoms are optionally and independently replaced with F.

In some embodiments, Q is Q1,

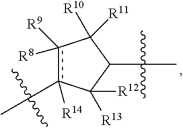

and [symbol] is a single bond and the compound of Formula I has the following structure:

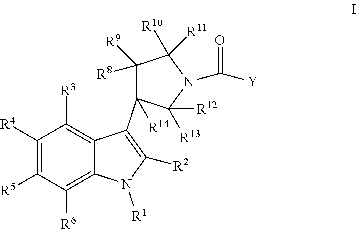

or a pharmaceutically acceptable salt and/or solvate thereof,
wherein:
R¹, R², R³, R⁴, R⁵, R⁶, R⁸, R⁹, R¹⁰, R¹¹, R¹², R¹³, R¹⁴ and Y are as defined for Formula I, and all available hydrogen atoms are optionally and independently replaced with a fluorine atom and/or all available hydrogen atoms are optionally and independently replaced substituted with deuterium.

In some embodiments, Q is Q1,

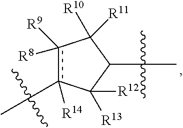

and [symbol] is a double bond and the compound of Formula I has the following structure:

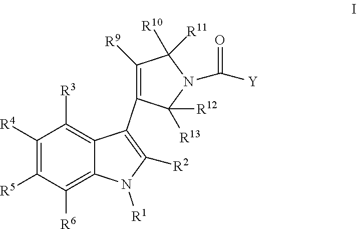

or a pharmaceutically acceptable salt and/or solvate thereof,
wherein:
R¹, R², R³, R⁴, R⁵, R⁶, R⁹, R¹⁰, R¹¹, R¹², R¹³ and Y are as defined for Formula I, and
all available hydrogen atoms are optionally and independently replaced with a fluorine atom and/or all available hydrogen atoms are optionally and independently replaced substituted with deuterium.

In some embodiments, Q is Q2,

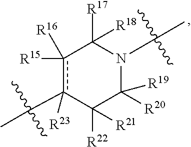

and [symbol] is a single bond and the compound of Formula I has the following structure:

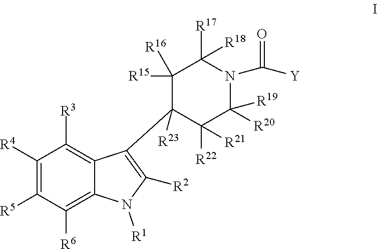

or a pharmaceutically acceptable salt and/or solvate thereof,
wherein:
R¹, R², R³, R⁴, R⁵, R⁶, R¹⁵, R¹⁶, R¹⁷, R¹⁸, R¹⁹, R²⁰, R²¹, R²², R²³ and Y are as defined for Formula I, provided that when Q is Q2, [symbol] is a single bond and R¹⁵, R¹⁶, R¹⁷, R¹⁸, R¹⁹, R²⁰, R²¹, R²² and R²³ are all H, then either (i) Y is selected from O—R⁴⁹ and O—C₁₋₄alkylene-O—C(O)—R⁴⁹, or (ii) the compound of Formula I comprises at least one deuterium; and
all available hydrogen atoms are optionally and independently replaced with a fluorine atom and/or all available hydrogen atoms are optionally and independently replaced substituted with deuterium.

In some embodiments, Q is Q2,

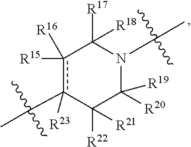

and [symbol] is a double bond and the compound of Formula I has the following structure:

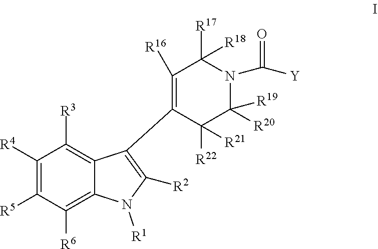

or a pharmaceutically acceptable salt and/or solvate thereof,
wherein:
R¹, R², R³, R⁴, R⁵, R⁶, R¹⁶, R¹⁷, R¹⁸, R¹⁹, R²⁰, R²¹, R²² and Y are as defined for Formula I, and all available hydrogen atoms are optionally and independently replaced with a fluorine atom and/or all available hydrogen atoms are optionally and independently replaced substituted with deuterium.

In some embodiments, Q is Q2 and [symbol] is a single bond and the compound of Formula I has the following structure:

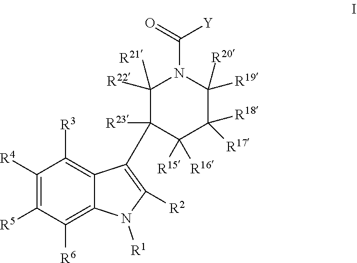

or a pharmaceutically acceptable salt and/or solvate thereof,
wherein:
R¹, R², R³, R⁴, R⁵, R⁶, R^{15′}, R¹⁶, R^{17′}, R^{18′}, R^{19′}, R^{20′}, R^{21′}, R^{22′} and R^{23′} are as defined for Formula I, and
all available hydrogen atoms are optionally and independently replaced with a fluorine atom and/or all available hydrogen atoms are optionally and independently replaced substituted with deuterium.

In some embodiments, Q is Q2′ and [symbol] is a double bond and the compound of Formula I has the following structure:

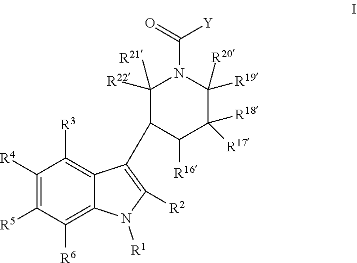

or a pharmaceutically acceptable salt and/or solvate thereof,
wherein:
R¹, R², R³, R⁴, R⁵, R⁶, R^{16′}, R^{17′}, R^{18′}, R^{19′}, R^{20′}, R^{21′} and R^{22′} are as defined for Formula I, and
all available hydrogen atoms are optionally and independently replaced with a fluorine atom and/or all available hydrogen atoms are optionally and independently replaced substituted with deuterium.

In some embodiments, Q is Q3,

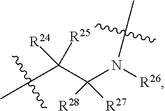

and the compound of Formula I has the following structure:

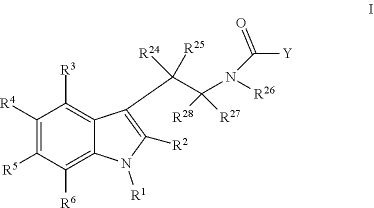

or a pharmaceutically acceptable salt and/or solvate thereof,
wherein:
R¹, R², R³, R⁴, R⁵, R⁶, R²⁴, R²⁵, R²⁶, R²⁷, R²⁸ and Y are as defined for Formula I provided that either (i) R²⁶ is C₁₋₆alkyl or (ii) Y is selected from —O—R⁴⁹ and —O—C₁₋₄alkylene-O—C(O)—R⁴⁹, or (iii) the compound of Formula I comprises at least one deuterium, and
all available hydrogen atoms are optionally and independently replaced with a fluorine atom and/or all available hydrogen atoms are optionally and independently replaced substituted with deuterium.

In some embodiments, Q is Q4,

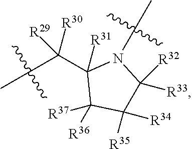

and the compound of Formula I has the following structure:

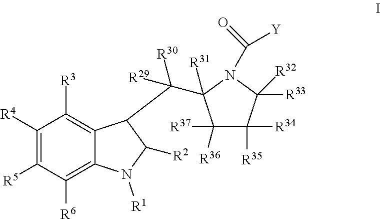

or a pharmaceutically acceptable salt and/or solvate thereof,
wherein:
R¹, R², R³, R⁴, R⁵, R⁶, R²⁹, R³⁰, R³¹, R³², R³³, R³⁴, R³⁵, R³⁶, R³⁷ and Y are as defined for Formula I, and
all available hydrogen atoms are optionally and independently replaced with a fluorine atom and/or all available hydrogen atoms are optionally and independently replaced substituted with deuterium.

In some embodiments, Q is Q5

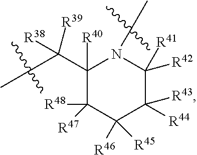

and the compound of Formula I has the following structure:

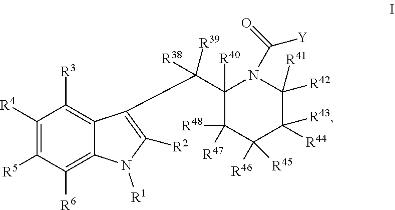

or a pharmaceutically acceptable salt and/or solvate thereof,
wherein:
R¹, R², R³, R⁴, R⁵, R⁶, R³⁸, R³⁹, R⁴⁰, R⁴¹, R⁴², R⁴³, R⁴⁴, R⁴⁵, R⁴⁶, R⁴⁷, R⁴⁸ and Y, are as defined for Formula I, and
all available hydrogen atoms are optionally and independently replaced with a fluorine atom and/or all available hydrogen atoms are optionally and independently replaced substituted with deuterium.

In some embodiments, R¹, R², R³, R⁵ and R⁶ are all H and the compound of Formula I has the following structure:

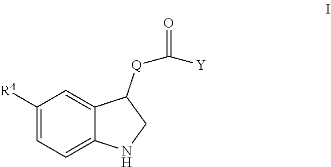

or a pharmaceutically acceptable salt and/or solvate thereof,
wherein:
R⁴, Q and Y are as defined for Formula I, and
all available hydrogen atoms are optionally and independently replaced with a fluorine atom and/or all available hydrogen atoms are optionally and independently replaced substituted with deuterium.

In some embodiments, R¹ and R² are both H and R³, R⁴, R⁵ and R⁶ are all D and the compound of Formula I has the following structure:

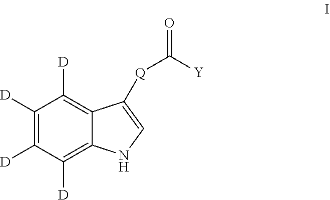

or a pharmaceutically acceptable salt and/or solvate thereof,
wherein:
Q and Y are as defined for Formula I, and
all available hydrogen atoms are optionally and independently replaced with a fluorine atom and/or all available hydrogen atoms are optionally and independently replaced substituted with deuterium.

In some embodiments, Y is selected from R⁴⁹, O—R⁴⁹ and O—C₁₋₄alkylene-O—C(O)—R⁴⁹, wherein all available hydrogen atoms are optionally and independently replaced with a fluorine atom or deuterium atom. In some embodiments, Y is selected from R⁴⁹, O—R⁴⁹ and O—C₁alkylene-O—C(O)—R⁴⁹, wherein all available hydrogen atoms are optionally and independently replaced with a fluorine atom or deuterium atom.

In some embodiments, R⁴⁹ is C₁₀₋₂₅alkyl, wherein all available hydrogen atoms are optionally and independently replaced with a fluorine and/or chlorine or deuterium atom. In some embodiments, R⁴⁹ is C₁₃₋₂₁alkyl, wherein all available hydrogen atoms are optionally and independently replaced with a fluorine atom or deuterium atom.

In some embodiments, R⁴⁹ is C₁₀₋₂₅alkenyl, wherein all available hydrogen atoms are optionally and independently replaced with a fluorine and/or chlorine or deuterium atom. In some embodiments, R⁴⁹ is C₁₃₋₂₁alkenyl, wherein all available hydrogen atoms are optionally and independently replaced with a fluorine atom or deuterium atom. In some embodiments, R⁴⁹ is C₁₀₋₂₅alkenyl and comprises 1, 2, 3, 4, 5 or 6 double bonds.

In some embodiments, the alkyl or alkene group of R⁴⁹ is an alkyl or alkenyl group present in a fatty acid, wherein all available H atoms are optionally substituted with deuterium. In some embodiments, R⁴¹ is an alkenyl group present in a fatty acid, wherein all available H atoms are optionally substituted with deuterium. In some embodiments, the fatty acid is an omega-6 fatty (i.e. an unsaturated or polyunsaturated fatty acid wherein the double bond that is closest to the methyl end of the molecule is located at carbon numbered 6 starting from the end methyl group)acid or an omega-3 fatty acid (i.e. an unsaturated or polyunsaturated fatty acid wherein the double bond that is closest to the methyl end of the molecule is located at carbon numbered 3 starting from the end methyl group), wherein all available H atoms are optionally substituted with deuterium. In some embodiments, R⁴⁹ is an alkyl group present in a fatty acid wherein all available H atoms are optionally substituted with deuterium. In some embodiments, the alkyl or alkene group of R⁴⁹ is an alkyl or alkene group present in a fatty acid selected from:

| | Lipid | |
|---|---|---|
| Common Name | Number | Chemical Name |
| linoleic acid (LA) | 18:2 (n-6) | all-cis-9,12-octadecadienoic |
| | | acid, |
| rumenic acid | 18:2 (n-6) | 9Z,11E-octadecadienoic |
| (conjugated linoleic acid) | | acid, |
| conjugated linoleic acid | 18:2 (n-6) | 10E,12Z-octadecadienoic |
| | | acid, |
| conjugated linoleic acid | 18:2 (n-6) | 9Z,12E-octadecadienoic |
| | | acid, |
| gamma-linolenic acid (GLA) | 18:3 (n-6) | all-cis-6,9,12- |
| | | octadecatrienoic acid, |
| calendic acid | 18:3 (n-6) | 8E,10E,12Z- |
| | | octadecatrienoic acid, |
| eicosadienoic acid | 20:2 (n-6) | all-cis-11,14-eicosadienoic |
| | | acid, |
| dihomo-gamma-linolenic | 20:3 (n-6) | all-cis-8,11,14- |
| acid (DGLA) | | eicosatrienoic acid, |
| arachidonic acid (AA, ARA) | 20:4 (n-6) | all-cis-5,8,11,14- |
| | | eicosatetraenoic acid |
| docosadienoic acid | 22:2 (n-6) | all-cis-13,16-docosadienoic |
| | | acid, |
| adrenic acid | 22:4 (n-6) | all-cis-7,10,13,16- |
| | | docosatetraenoic acid, |
| osbond acid | 22:5 (n-6) | all-cis-4,7,10,13,16- |
| | | docosapentaenoic acid, |
| tetracosatetraenoic acid | 24:4 (n-6) | all-cis-9,12,15,18- |
| | | tetracosatetraenoic acid, |
| tetracosapentaenoic acid | 24:5 (n-6) | all-cis-6,9,12,15,18- |
| | | tetracosapentaenoic acid, |
| α-linolenic acid (ALA) | 18:3 (n-3) | all-cis-9,12,15- |
| | | octadecatrienoic acid, |
| stearidonic acid (SDA) | 18:4 (n-3) | all-cis-6,9,12,15- |
| | | octadecatetraenoic acid, |
| hexadecatrienoic acid | 16:3 (n-3) | all-cis-7,10,13- |
| (HTA) | | hexadecatrienoic acid, |
| eicosatrienoic acid (ETE) | 20:3 (n-3) | all-cis-11,14,17- |
| | | eicosatrienoic acid, |
| eicosatetraenoic acid (ETA) | 20:4 (n-3) | all-cis-8,11,14,17- |
| | | eicosatetraenoic acid, |
| eicosapentaenoic acid | 20:5 (n-3) | all-cis-5,8,11,14,17- |
| (EPA) | | eicosapentaenoic acid, |
| heneicosapentaenoic acid | 21:5 (n-3) | all-cis-6,9,12,15,18- |
| (HPA) | | heneicosapentaenoic acid, |
| docosapentaenoic acid | 22:5 (n-3) | all-cis-7,10,13,16,19- |
| (DPA) | | docosapentaenoic acid, |
| docosahexaenoic acid | 22:6 (n-3) | all-cis-4,7,10,13,16,19- |
| (DHA) | | docosahexaenoic acid, |
| tetracosapentaenoic acid | 24:5 (n-3) | all-cis-9,12,15,18,21- |
| | | tetracosapentaenoic acid, |
| tetracosahexaenoic acid | 24:6 (n-3) | all-cis-6,9,12,15,18,21- |
| (Nisinic acid) | | tetracosahexaenoic acid, |
| myristoleic acid | 14:1 (n-5) | 9Z-tetradecenoic acid, |
| palmitoleic acid | 16:1 (n-7) | (9Z)-hexadecenoic acid, |
| sapienic acid | 16:1 (n-10) | (6Z)-hexadecenoic acid, |
| oleic acid | 18-1 (n-9) | (9Z)-octadecenoic acid, |
| elaidic acid | 18:1 (n-9) | (E)-octadecenoic acid, |
| vaccenic acid | 18:1 (n-7) | (11E)-octadecenoic acid, |
| eruric acid | 22-1 (n-9) | (13Z)-Docosenoic acid, |
| caprylic acid | 8:0 | octanoic acid, |
| capric acid | 10:0 | decanoic acid, |
| lauric acid | 12:0 | dodecanoic acid, |
| myristic acid | 14:0 | tetradecanoic acid, |
| palmitic acid | 16:0 | hexadecenoic acid, |
| stearic acid | 18:0 | octadecanoic acid, |
| arachidic acid | 20:0 | Icosanoic acid, |
| behenic acid | 22:0 | docosanoic acid, |
| lignoceric acid | 24:0 | tetracosanoic acid, and |
| cerotic acid | 26:0 | hexacosanoic acid, |

wherein all available H atoms are optionally substituted with deuterium.

In some embodiments, alkene group of R⁴⁹ is an alkyl or alkenyl group present in linoleic acid, docosadienoic acid or eicosadienoic acid. In some embodiments, alkene group of R⁷ is an alkyl or alkenyl group present in linoleic acid.

In some embodiments, when R⁴⁹ is the alkyl or alkenyl group of a fatty acid wherein 1-10, 2-8, 2-6 or 2-4H atoms are substituted with deuterium.

In some embodiments, R⁴⁹ is (CH₂)₇CH═CH(CH₂)₇CH₃. In some embodiments, R⁴⁹ is (CH₂)₇CH═CHCH₂CH═CH(CH₂)₄CH₃. In some embodiments, R⁴⁹ is (CH₂)₈CH═CHCH₂CH═CH(CH₂)₄CH₃. In some embodiments, R⁴⁹ is (CH₂)₇CH═CHCH₂CH═CHCH₂CH═CH(CH₂)₁CH₃. In some embodiments, R⁴⁹ is (CH₂)₃CH═CHCH₂CH═CH(CH₂)₁CH═CHCH₂CH═CH(CH₂)₃CH₃. In some embodiments, R⁴⁹ is (CH₂)₂CH═CHCH₂CH═CHCH₂CH═CHCH₂CH═CHCH₂CH═CHCH₂CH═CH(CH₂)₁CH₃.

In some embodiments, Y is R⁴⁹.

In some embodiments, Y is —O—R⁴⁹.

In some embodiments, Y is —O—C₃alkylene-O—C(O)—R⁴⁹.

In some embodiments, the compound of Formula I has the following structure:

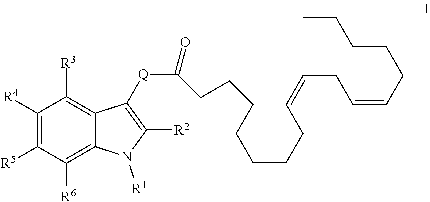

or a pharmaceutically acceptable salt and/or solvate thereof,
wherein:
R¹, R², R³, R⁴, R⁵, R⁶ and Q are as defined for Formula I, and
all available hydrogen atoms are optionally and independently replaced with a fluorine atom and/or all available hydrogen atoms are optionally and independently replaced with deuterium.

In some embodiments, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, D, F, Cl, C₁₋₆alkyl, C₁₋₆fluoroalkyl and C₁₋₆deuteroalkyl. In some embodiments, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, F, D, CH₃, CD₂H, CDH₂, CD₃, CF₃, CHF₂, CH₂CH₃, CH₂CH₂D, CH₂CD₂H and CD₂CD₃. In some embodiments, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, F, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H and D.

In some embodiments, R^{15′}, R^{16′}, R^{17′}, R^{18′}, R^{19′}, R^{20′}, R^{21′}, R^{22′} and R^{23′} are independently selected from H, D, F, Cl, C₁₋₆alkyl, C₁₋₆fluoroalkyl and C₁₋₆deuteroalkyl. In some embodiments, R^{15′}, R^{16′}, R^{17′}, R^{18′}, R^{19′}, R^{20′}, R^{21′}, R^{22′} and R^{23′} are independently selected from H, F, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{15′}, R^{16′}, R^{17′}, R^{18′}, R^{19′}, R^{20′}, R^{21′}, R^{22′} and R^{23′} are independently selected from H and D. In some embodiments, when Q is

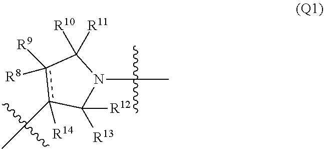

and [symbol] in Q1 is a single bond or Q is

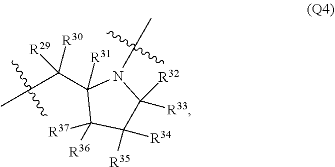

the stereochemistry at the carbon to which R¹⁴ or R³¹ is bonded is either R or S. Therefore in some embodiments, Q1 is

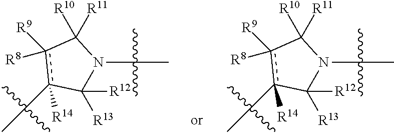

### and Q4 is

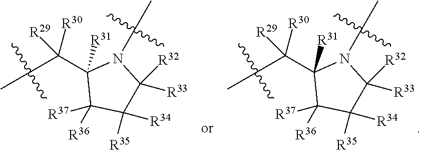

In some embodiments, the stereochemistry at the carbon to which R¹⁴ or R³¹ is bonded is R. In some embodiments, the stereochemistry at the carbon to which R¹⁴ or R³¹ is bonded is S.

In some embodiments, when Q is

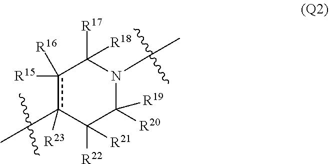

and [symbol] in Q2 is a single bond, the stereochemistry at the carbon to which R²³ is bonded is either R or S. Therefore in some embodiments Q2 is

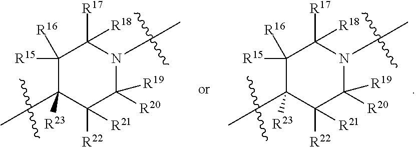

In some embodiments, when Q is

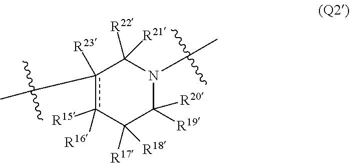

and [symbol] in Q2′ is a single bond, the stereochemistry at the carbon to which R^{23′} is bonded is either R or S. Therefore in some embodiments Q2′ is

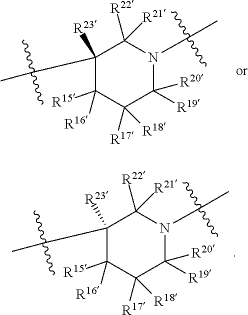

In some embodiments, when Q is Q5

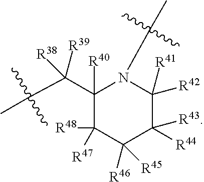

the stereochemistry at the carbon to which R⁴⁰ is bonded is either R or S. Therefore in some embodiments, Q5 is

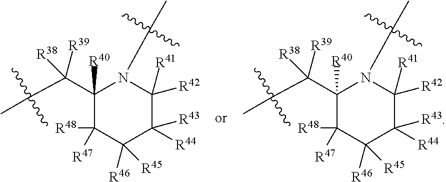

In some embodiments, Q is selected from one of the following groups:

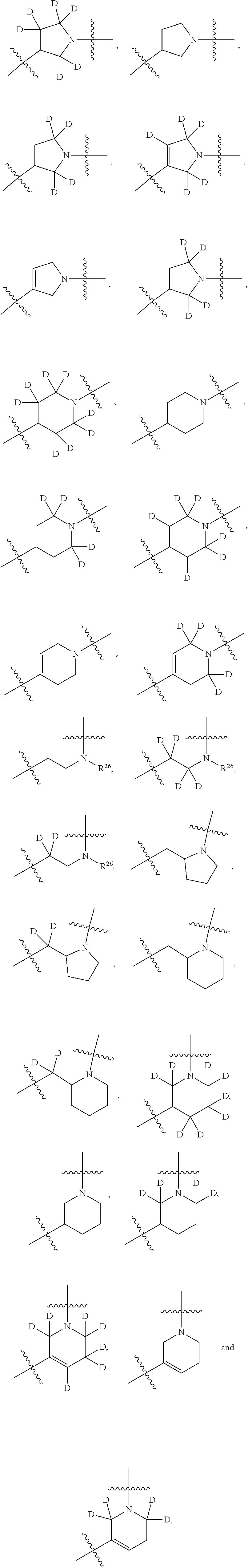

wherein R²⁶ is selected from H, O, C₁₋₆alkyl, C₁fluoroalkyl and C₁₋₆deuteroalkyl. In some embodiments, R²⁶ is selected from H, C₁₋₄alkyl, C₁₋₄fluoroalkyl and C₁₋₄deuteroalkyl. In some embodiments, R²⁶ is selected from H, CH₃, CD₃, CF₂H and CF₃. In some embodiments, R²⁶ is selected from H and CH₃. In some embodiments, R²⁶ is CH(CH₃)₂.

In some embodiments Q is selected from one of the following groups:

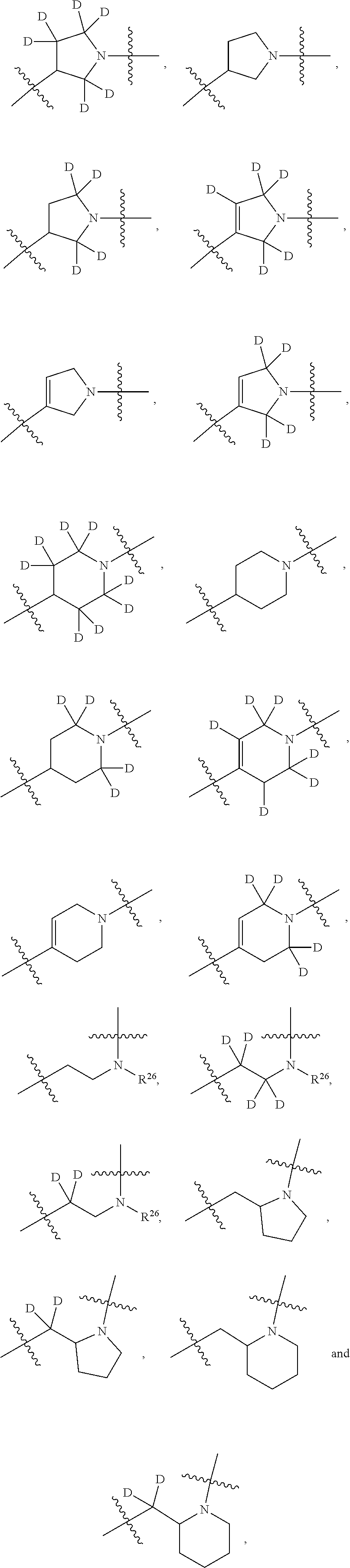

wherein R²⁶ is selected from H, D, C₁₋₆alkyl, C₁₋₆fluoroalkyl and C₁₋₆deuteroalkyl. In some embodiments, R²⁶ is selected from H, C₁₋₄alkyl, C₁₋₄fluoroalkyl and C₁₋₄deuteroalkyl. In some embodiments, R²⁶ is selected from H, CH₃, CD₃, CF₂H and CF₃. In some embodiments, R²⁶ is selected from H and CH₃.

In some embodiments R¹ is H.

In some embodiments, R² is selected from H, halo, C₁₋₄alkyl and C₁₋₄alkoxy. In some embodiments, R² is selected from H, D, F, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R² is selected from H, D, F, Cl, CH₃, CF₃, CF₂H, CD₃, CH₃O, CF₃O, CHF₂O, and CD₃O. In some embodiments, R² is selected from H, D, F, Cl, CH₃, CH₃CH₂, CH₃CH₂O, (CH₃)₂CH, (CH₃)₂CHO, CF₃, CF₂H, CD₃, CH₃O, CF₃O, CHF₂O, and CD₃O. In some embodiments, R² is H or D. In some embodiments, R² is H or D.

In some embodiments, R², R⁵ and R⁶ are independently H, D or F. In some embodiments, R², R⁵ and R⁶ are all H or are all D. In some embodiments, R², R⁵ and R⁶ are all H. In some embodiments, R², R⁵ and R⁶ are independently selected from H, D, F, CH₃ and CD₃.

In some embodiments, R³ is selected from H, D, F, OH, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R³ is selected from H, D, F, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R³ is selected from H, D, F, C, OH, CH₃, CF₃, CF₂H, CD₃, CH₃O, CF₃O, CHF₂O, and CD₃O. In some embodiments, R³ is selected from H, D, F, C, CH₃, CF₃, CF₂H, CD₃, CH₃O, CF₃O, CHF₂O, and CD₃O. In some embodiments, R³ is selected from H, D, F, C, CH₃, CH₃CH₂, CH₃CH₂O, (CH₃)₂CH, (CH₃)₂CHO, CF₃, CF₂H, CD₃, CH₃O, CF₃O, CHF₂O, and CD₃O In some embodiments, R³ is selected from H, D, OH and CH₃O. In some embodiments, R³ is selected from H, D and CH₃O. In some embodiments, R³ is H.

In some embodiments, R⁴ is selected from H, D, F, OH, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R⁴ is selected from H, D, F, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R⁴ is selected from H, D, F, C, OH, CH₃, CF₃, CF₂H, CD₃, CH₃O, CF₃O, CHF₂O, and CD₃O. In some embodiments, R⁴ is selected from H, D, F, Cl, CH₃, CH₃CH₂, CH₃CH₂O, (CH₃)₂CH, (CH₃)₂CHO, CF₃, CF₂H, CD₃, CH₃O, CF₃O, CHF₂O, and CD₃O. In some embodiments, R⁴ is selected from H, D, F, Cl, CH₃, CF₃, CF₂H, CD₃, CH₃O, CF₃O, CHF₂O, and CD₃O. In some embodiments, R⁴ is CH₃O. In some embodiments, R⁴ is H.

In some embodiments, one of R³ and R⁴ is OH.

In some embodiments, one or both of R³ and R⁴ is independently selected from H, D, F, Cl, CH₃, CH₂CH₃, CH₂CH₂CH₃, CH(CH₃)₂, CF₃, CF₂H, CH₂CF₂H, CH₂CF₃, CH₂CFH₂, CH(CF₃)₂, CD₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, and CD₃O. In some embodiments, one or both of R³ and R⁴ is independently selected from H, D, F, Cl, CH₃, CH(CH₃)₂, CF₃, CF₂H, CD₃, CH(CH₃)₂O, CH₃O, CF₃O, CHF₂O, and CD₃O.

In some embodiments both of R³ and R⁴ are independently selected from D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments both of R³ and R⁴ are independently selected from D, F, Cl, CH₃, CH(CH₃)₂, CF₃, CF₂H, CD₃, CH(CH₃)₂O, CH₃O, CF₃O, CHF₂O, and CD₃O.

In some embodiments, R³ is H or D and R⁴ is selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R³ is H or D and R⁴ is selected from D, F, C, CH₃, CH(CH₃)₂, CF₃, CF₂H, CD₃, CH(CH₃)₂O, CH₃O, CF₃O, CHF₂O, and CD₃O.

In some embodiments, R⁴ is H or D and R³ is selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R⁴ is H or D and R³ is selected from D, F, C, CH₃, CH(CH₃)₂, CF₃, CF₂H, CD₃, CH(CH₃)₂O, CH₃O, CF₃O, CHF₂O, and CD₃O.

In some embodiments, one of R³ and R⁴ is OP(O)(OR⁵⁰)₂, and the other of R³ and R⁴ is selected from H, halo, C₁₋₄alkyl, C₁₋₄fluoroalkyl and C₁₋₄deuteroalkyl. In some embodiments, one of R³ and R⁴ is OP(O)(OR⁵⁰)₂, and the other of R³ and R⁴ is selected from H, C, F, C₁₋₂alkyl, C₁₋₄fluoroalkyl and C₁₋₂deuteroalkyl. In some embodiments, one of R³ and R⁴ is OP(O)(OR⁵⁰)₂, and the other of R³ and R⁴ is H.

In some embodiments, R⁵⁰ is selected from H, C₁₋₄alkyl, C₁₋₄fluoroalkyl and C₁₋₄deuteroalkyl. In some embodiments, R⁵⁰ is selected from H, C₁₋₂alkyl, C₁₋₂fluoroalkyl and C₁₋₂deuteroalkyl. In some embodiments, R⁵⁰ is selected from H, D, CH₃, CF₃, CF₂H and CD₃.

In some embodiments, the compounds of Formula I are selected from the compounds listed below, or a pharmaceutically acceptable salt, solvate and/or prodrug thereof:

| Compound I.D | Chemical Structure |
|---|---|
| I-1 | 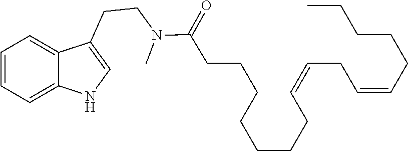 [SMILES: CCCCC/C=C\C/C=C\CCCCCCCC(=O)N(C)CCc1c[nH]c2ccccc12] |
| I-2 | 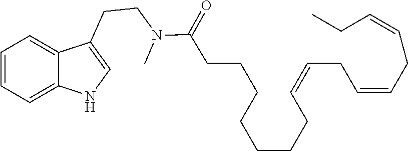 [SMILES: CC/C=C\C/C=C\C/C=C\CCCCCCCC(=O)N(C)CCc1c[nH]c2ccccc12] |
| I-3 | 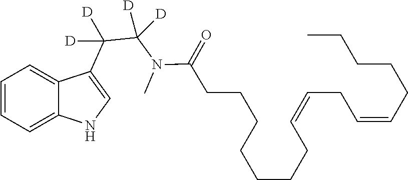 [SMILES: [2H]C([2H])(c1c[nH]c2ccccc12)C([2H])([2H])N(C)C(=O)CCCCCCC/C=C\C/C=C\CCCCC] |
| I-4 | 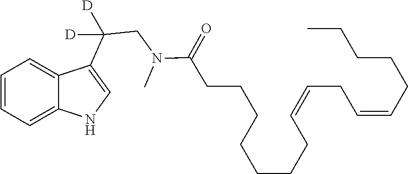 [SMILES: [2H]C([2H])(CN(C)C(=O)CCCCCCC/C=C\C/C=C\CCCCC)c1c[nH]c2ccccc12] |
| I-5 | 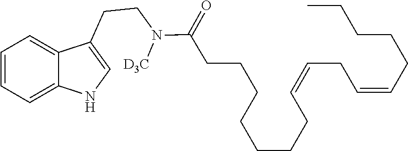 [SMILES: [2H]C([2H])([2H])N(CCc1c[nH]c2ccccc12)C(=O)CCCCCCC/C=C\C/C=C\CCCCC] |
| I-6 | 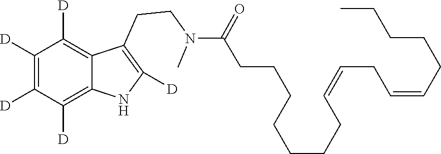 [SMILES: [2H]c1[nH]c2c([2H])c([2H])c([2H])c([2H])c2c1CCN(C)C(=O)CCCCCCC/C=C\C/C=C\CCCCC] |
| I-7 | 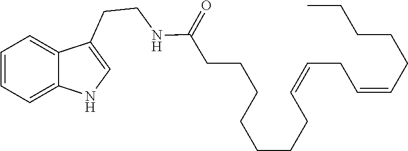 [SMILES: CCCCC/C=C\C/C=C\CCCCCCCC(=O)NCCc1c[nH]c2ccccc12] |
| I-8 | 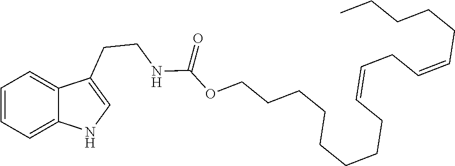 [SMILES: CCCCC/C=C\C/C=C\CCCCCCCCOC(=O)NCCc1c[nH]c2ccccc12] |
| I-9 | 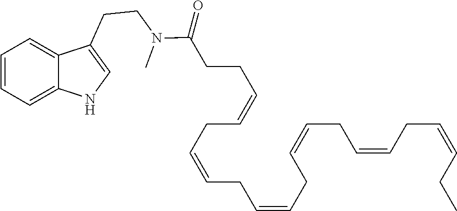 [SMILES: CC/C=C\C/C=C\C/C=C\C/C=C\C/C=C\C/C=C\CCC(=O)N(C)CCc1c[nH]c2ccccc12] |
| I-10 | 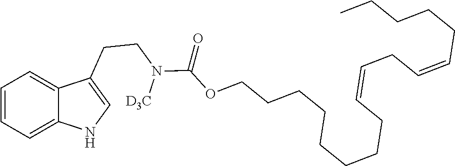 [SMILES: [2H]C([2H])([2H])N(CCc1c[nH]c2ccccc12)C(=O)OCCCCCCCC/C=C\C/C=C\CCCCC] |
| I-11 | 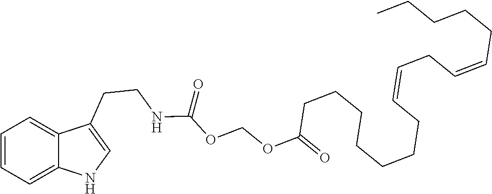 [SMILES: CCCCC/C=C\C/C=C\CCCCCCCC(=O)OCOC(=O)NCCc1c[nH]c2ccccc12] |
| I-12 | 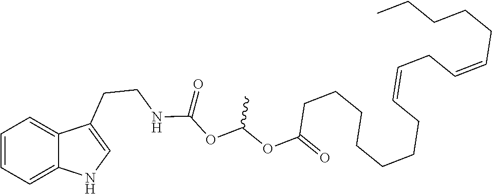 [SMILES: CCCCC/C=C\C/C=C\CCCCCCCC(=O)OC(C)OC(=O)NCCc1c[nH]c2ccccc12] |
| I-13 | 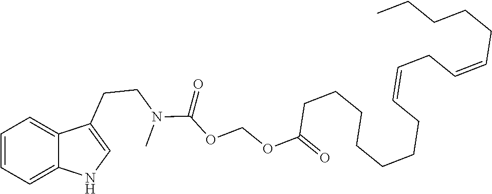 [SMILES: CCCCC/C=C\C/C=C\CCCCCCCC(=O)OCOC(=O)N(C)CCc1c[nH]c2ccccc12] |
| I-14 | 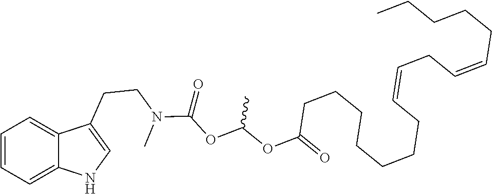 [SMILES: CCCCC/C=C\C/C=C\CCCCCCCC(=O)OC(C)OC(=O)N(C)CCc1c[nH]c2ccccc12] |
| I-15 | 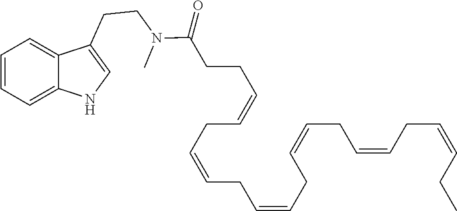 [SMILES: CC/C=C\C/C=C\C/C=C\C/C=C\C/C=C\C/C=C\CCC(=O)N(C)CCc1c[nH]c2ccccc12] |
| I-16 | 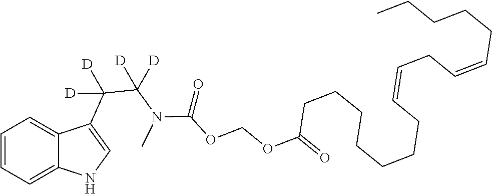 [SMILES: [2H]C([2H])(c1c[nH]c2ccccc12)C([2H])([2H])N(C)C(=O)OCOC(=O)CCCCCCC/C=C\C/C=C\CCCCC] |
| I-17 | 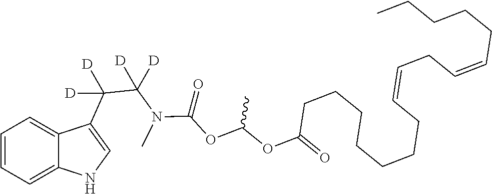 [SMILES: [2H]C([2H])(c1c[nH]c2ccccc12)C([2H])([2H])N(C)C(=O)OC(C)OC(=O)CCCCCCC/C=C\C/C=C\CCCCC] |
| I-18 | 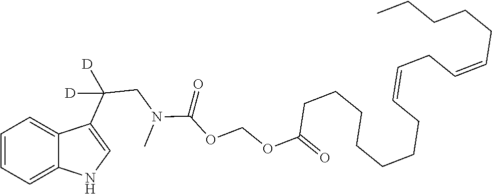 [SMILES: [2H]C([2H])(CN(C)C(=O)OCOC(=O)CCCCCCC/C=C\C/C=C\CCCCC)c1c[nH]c2ccccc12] |
| I-19 | 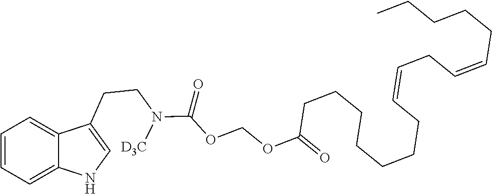 [SMILES: [2H]C([2H])([2H])N(CCc1c[nH]c2ccccc12)C(=O)OCOC(=O)CCCCCCC/C=C\C/C=C\CCCCC] |
| I-20 | 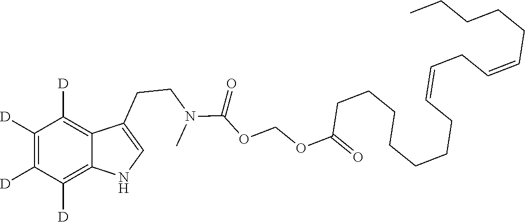 [SMILES: [2H]c1c([2H])c([2H])c2c(CCN(C)C(=O)OCOC(=O)CCCCCCC/C=C\C/C=C\CCCCC)c[nH]c2c1[2H]] |
| I-21 | 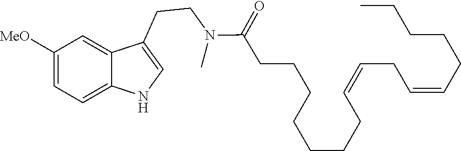 [SMILES: CCCCC/C=C\C/C=C\CCCCCCCC(=O)N(C)CCc1c[nH]c2ccc(OC)cc12] |
| I-22 | 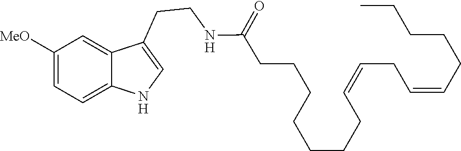 [SMILES: CCCCC/C=C\C/C=C\CCCCCCCC(=O)NCCc1c[nH]c2ccc(OC)cc12] |
| I-23 | 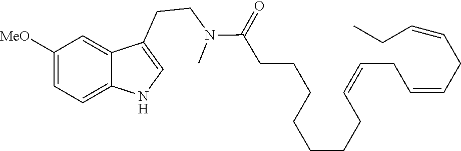 [SMILES: CC/C=C\C/C=C\C/C=C\CCCCCCCC(=O)N(C)CCc1c[nH]c2ccc(OC)cc12] |
| I-24 | 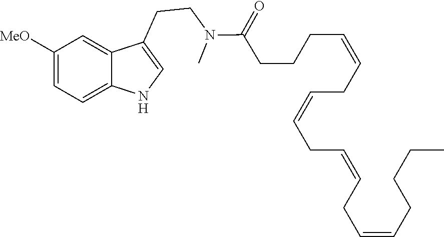 [SMILES: CCCC/C=C\C/C=C\C/C=C\C/C=C\CCCC(=O)N(C)CCc1c[nH]c2ccc(OC)cc12] |
| I-25 | 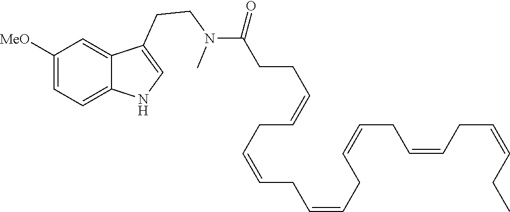 [SMILES: CC/C=C\C/C=C\C/C=C\C/C=C\C/C=C\C/C=C\CCC(=O)N(C)CCc1c[nH]c2ccc(OC)cc12] |
| I-26 | 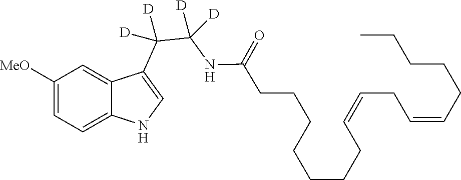 [SMILES: [2H]C([2H])(NC(=O)CCCCCCC/C=C\C/C=C\CCCCC)C([2H])([2H])c1c[nH]c2ccc(OC)cc12] |
| I-27 | 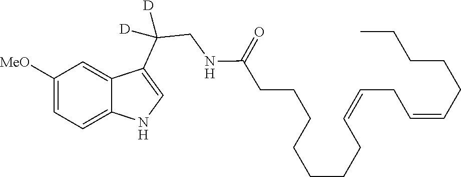 [SMILES: [2H]C([2H])(CNC(=O)CCCCCCC/C=C\C/C=C\CCCCC)c1c[nH]c2ccc(OC)cc12] |
| I-28 | 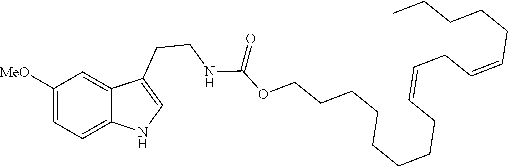 [SMILES: CCCCC/C=C\C/C=C\CCCCCCCCOC(=O)NCCc1c[nH]c2ccc(OC)cc12] |
| I-29 | 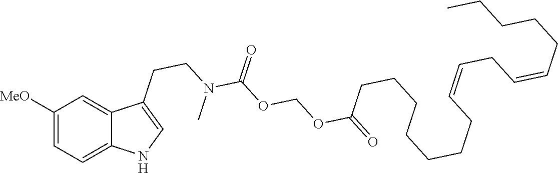 [SMILES: CCCCC/C=C\C/C=C\CCCCCCCC(=O)OCOC(=O)N(C)CCc1c[nH]c2ccc(OC)cc12] |
| I-30 | 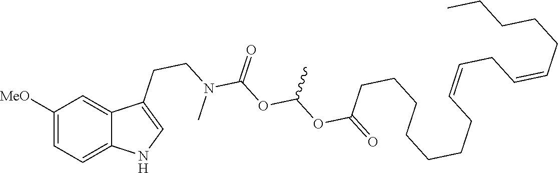 [SMILES: CCCCC/C=C\C/C=C\CCCCCCCC(=O)OC(C)OC(=O)N(C)CCc1c[nH]c2ccc(OC)cc12] |
| I-31 | 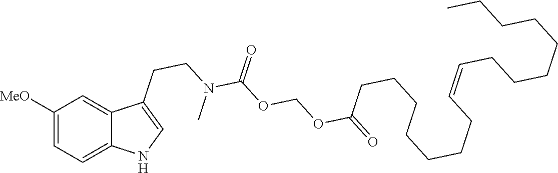 [SMILES: CCCCCCCC/C=C\CCCCCCCC(=O)OCOC(=O)N(C)CCc1c[nH]c2ccc(OC)cc12] |
| I-32 | 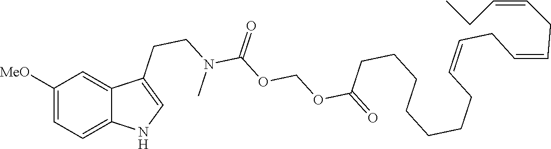 [SMILES: CC/C=C\C/C=C\C/C=C\CCCCCCCC(=O)OCOC(=O)N(C)CCc1c[nH]c2ccc(OC)cc12] |
| I-33 | 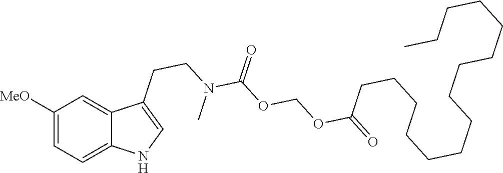 [SMILES: CCCCCCCCCCCCCCCC(=O)OCOC(=O)N(C)CCc1c[nH]c2ccc(OC)cc12] |
| I-34 | 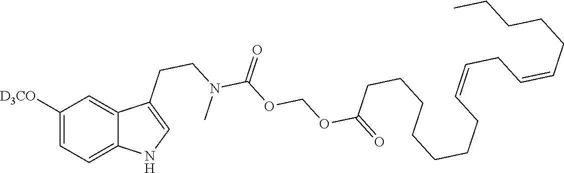 |
| I-35 | 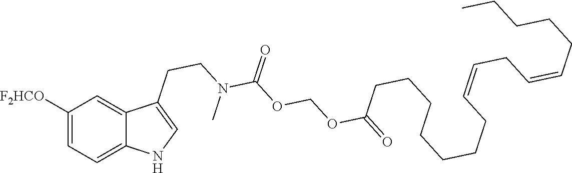 [SMILES: CCCCC/C=C\C/C=C\CCCCCCCC(=O)OCOC(=O)N(C)CCc1c[nH]c2ccc(OC(F)F)cc12] |
| I-36 | 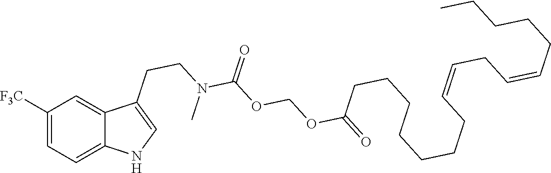 [SMILES: CCCCC/C=C\C/C=C\CCCCCCCC(=O)OCOC(=O)N(C)CCc1c[nH]c2ccc(C(F)(F)F)cc12] |
| I-37 | 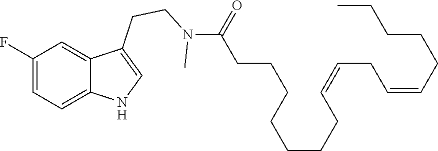 [SMILES: CCCCC/C=C\C/C=C\CCCCCCCC(=O)N(C)CCc1c[nH]c2ccc(F)cc12] |
| I-38 | 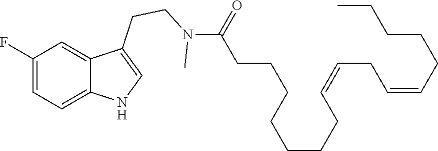 [SMILES: CCCCC/C=C\C/C=C\CCCCCCCC(=O)N(C)CCc1c[nH]c2ccc(F)cc12] |
| I-39 | 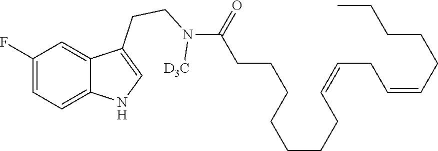 [SMILES: [2H]C([2H])([2H])N(CCc1c[nH]c2ccc(F)cc12)C(=O)CCCCCCC/C=C\C/C=C\CCCCC] |
| I-40 | 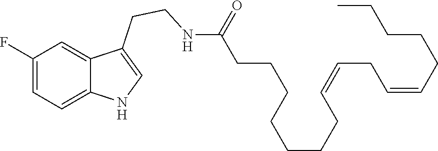 [SMILES: CCCCC/C=C\C/C=C\CCCCCCCC(=O)NCCc1c[nH]c2ccc(F)cc12] |
| I-41 | 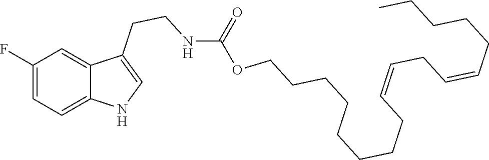 [SMILES: CCCCC/C=C\C/C=C\CCCCCCCCOC(=O)NCCc1c[nH]c2ccc(F)cc12] |
| I-42 | 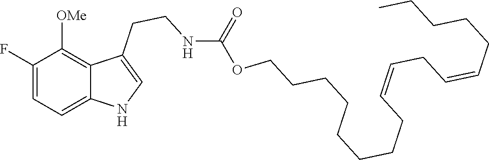 [SMILES: CCCCC/C=C\C/C=C\CCCCCCCCOC(=O)NCCc1c[nH]c2ccc(F)c(OC)c12] |
| I-43 | 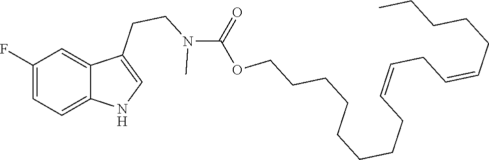 [SMILES: CCCCC/C=C\C/C=C\CCCCCCCCOC(=O)N(C)CCc1c[nH]c2ccc(F)cc12] |
| I-44 | 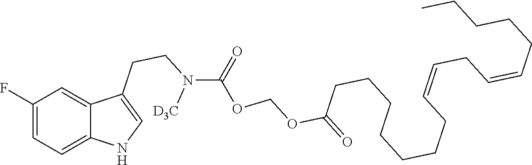 [SMILES: [2H]C([2H])([2H])N(CCc1c[nH]c2ccc(F)cc12)C(=O)OCOC(=O)CCCCCCC/C=C\C/C=C\CCCCC] |
| (R)-I-45 | 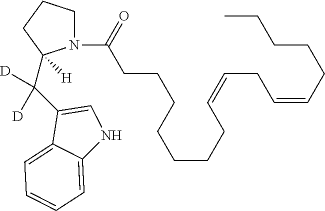 [SMILES: [2H]C([2H])(c1c[nH]c2ccccc12)[C@H]1CCCN1C(=O)CCCCCCC/C=C\C/C=C\CCCCC] |
| (R)-I-46 | 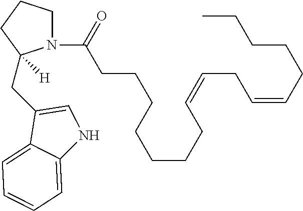 [SMILES: CCCCC/C=C\C/C=C\CCCCCCCC(=O)N1CCC[C@@H]1Cc1c[nH]c2ccccc12] |
| (R)-I-47 | 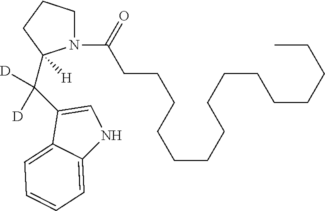 [SMILES: [2H]C([2H])(c1c[nH]c2ccccc12)[C@H]1CCCN1C(=O)CCCCCCCCCCCCCCC] |
| (R)-I-48 | 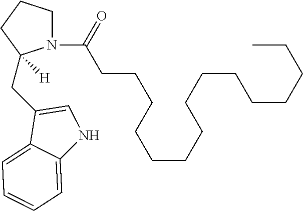 [SMILES: CCCCCCCCCCCCCCCC(=O)N1CCC[C@@H]1Cc1c[nH]c2ccccc12] |
| (R)-I-49 | 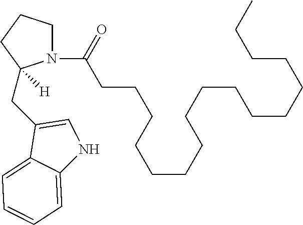 [SMILES: CCCCCCCCCCCCCCCCCC(=O)N1CCC[C@@H]1Cc1c[nH]c2ccccc12] |
| (R)-I-50 | 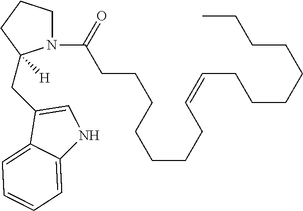 [SMILES: CCCCCCCC/C=C\CCCCCCCC(=O)N1CCC[C@@H]1Cc1c[nH]c2ccccc12] |
| (R)-I-51 | 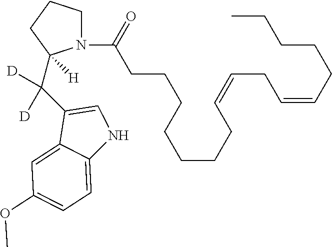 [SMILES: [2H]C([2H])(c1c[nH]c2ccc(OC)cc12)[C@H]1CCCN1C(=O)CCCCCCC/C=C\C/C=C\CCCCC] |
| (R)-I-52 | 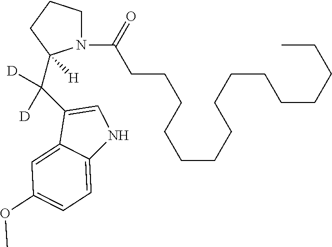 [SMILES: [2H]C([2H])(c1c[nH]c2ccc(OC)cc12)[C@H]1CCCN1C(=O)CCCCCCCCCCCCCCC] |
| (R)-I-53 | 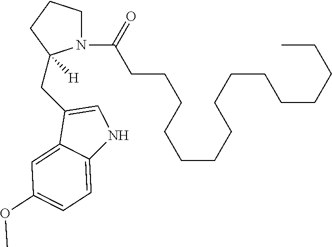 [SMILES: CCCCCCCCCCCCCCCC(=O)N1CCC[C@@H]1Cc1c[nH]c2ccc(OC)cc12] |
| (R)-I-54 | 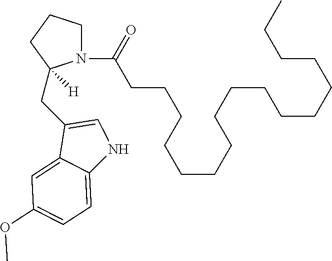 [SMILES: CCCCCCCCCCCCCCCCCC(=O)N1CCC[C@@H]1Cc1c[nH]c2ccc(OC)cc12] |
| (R)-I-55 | 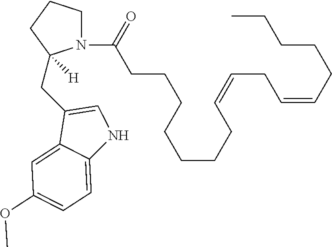 [SMILES: CCCCC/C=C\C/C=C\CCCCCCCC(=O)N1CCC[C@@H]1Cc1c[nH]c2ccc(OC)cc12] |
| (R)-I-56 | 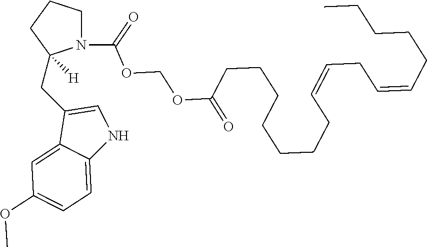 [SMILES: CCCCC/C=C\C/C=C\CCCCCCCC(=O)OCOC(=O)N1CCC[C@@H]1Cc1c[nH]c2ccc(OC)cc12] |
| (R)-I-57 | 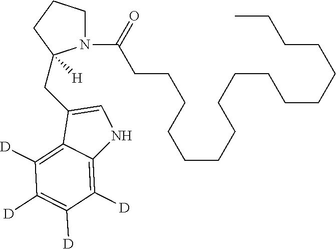 [SMILES: [2H]c1c([2H])c([2H])c2c(C[C@H]3CCCN3C(=O)CCCCCCCCCCCCCCCCC)c[nH]c2c1[2H]] |
| (R)-I-58 | 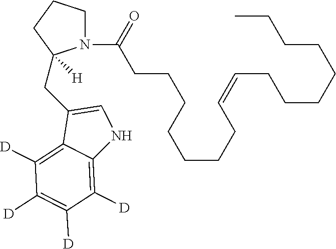 [SMILES: [2H]c1c([2H])c([2H])c2c(C[C@H]3CCCN3C(=O)CCCCCCC/C=C\CCCCCCCC)c[nH]c2c1[2H]] |
| (R)-I-59 | 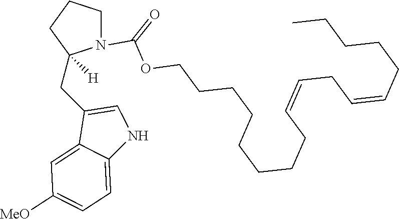 [SMILES: CCCCC/C=C\C/C=C\CCCCCCCCOC(=O)N1CCC[C@@H]1Cc1c[nH]c2ccc(OC)cc12] |
| (R)-I-60 | 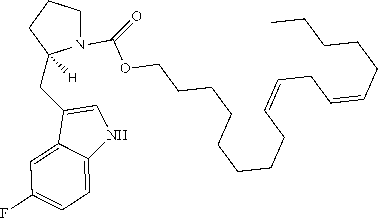 [SMILES: CCCCC/C=C\C/C=C\CCCCCCCCOC(=O)N1CCC[C@@H]1Cc1c[nH]c2ccc(F)cc12] |
| (R)-I-61 | 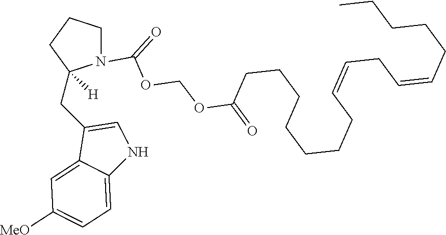 [SMILES: CCCCC/C=C\C/C=C\CCCCCCCC(=O)OCOC(=O)N1CCC[C@@H]1Cc1c[nH]c2ccc(OC)cc12] |
| (R)-I-62 | 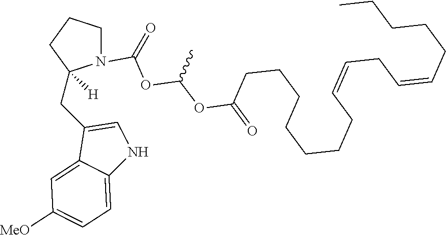 [SMILES: CCCCC/C=C\C/C=C\CCCCCCCC(=O)OC(C)OC(=O)N1CCC[C@@H]1Cc1c[nH]c2ccc(OC)cc12] |
| (R)-I-63 | 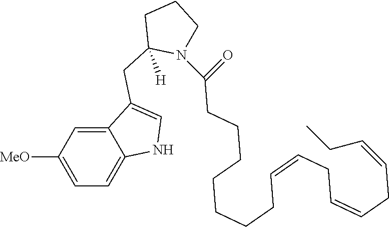 [SMILES: CC/C=C\C/C=C\C/C=C\CCCCCCCC(=O)N1CCC[C@@H]1Cc1c[nH]c2ccc(OC)cc12] |
| (S)-I-64 | 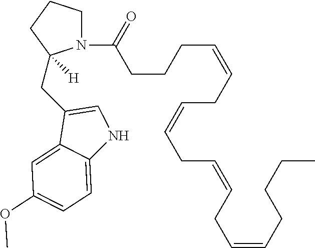 [SMILES: CCCC/C=C\C/C=C\C/C=C\C/C=C\CCCC(=O)N1CCC[C@@H]1Cc1c[nH]c2ccc(OC)cc12] |
| (R)-I-65 | 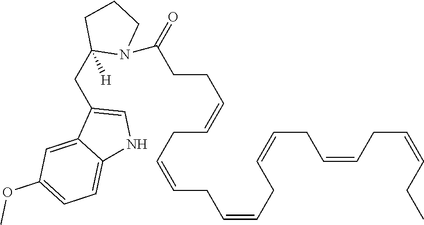 [SMILES: CC/C=C\C/C=C\C/C=C\C/C=C\C/C=C\C/C=C\CCC(=O)N1CCC[C@@H]1Cc1c[nH]c2ccc(OC)cc12] |
| (R)-I-66 | 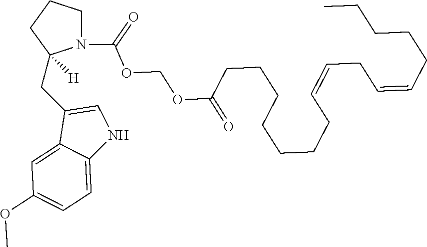 [SMILES: CCCCC/C=C\C/C=C\CCCCCCCC(=O)OCOC(=O)N1CCC[C@@H]1Cc1c[nH]c2ccc(OC)cc12] |
| (R)-I-67 | 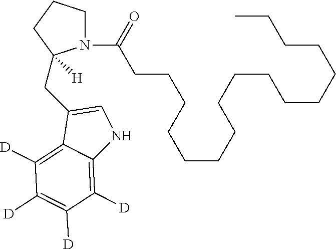 [SMILES: [2H]c1c([2H])c([2H])c2c(C[C@H]3CCCN3C(=O)CCCCCCCCCCCCCCCCC)c[nH]c2c1[2H]] |
| (R)-I-68 | 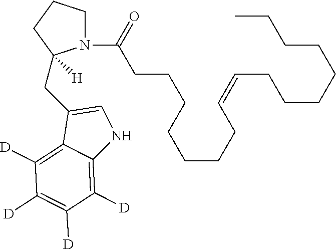 [SMILES: [2H]c1c([2H])c([2H])c2c(C[C@H]3CCCN3C(=O)CCCCCCC/C=C\CCCCCCCC)c[nH]c2c1[2H]] |
| (R)-I-69 | 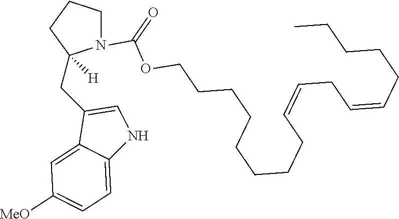 [SMILES: CCCCC/C=C\C/C=C\CCCCCCCCOC(=O)N1CCC[C@@H]1Cc1c[nH]c2ccc(OC)cc12] |
| (R)-I-70 | 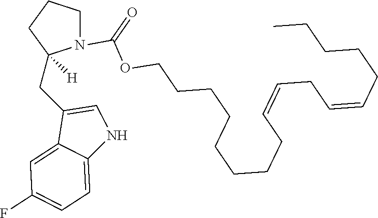 [SMILES: CCCCC/C=C\C/C=C\CCCCCCCCOC(=O)N1CCC[C@@H]1Cc1c[nH]c2ccc(F)cc12] |
| (R)-I-71 | 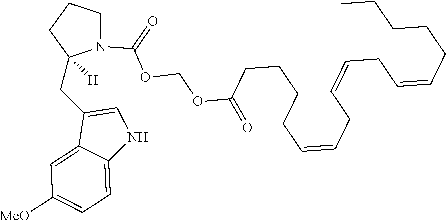 [SMILES: CCCCC/C=C\C/C=C\C/C=C\CCCCC(=O)OCOC(=O)N1CCC[C@@H]1Cc1c[nH]c2ccc(OC)cc12] |
| (R)-I-72 | 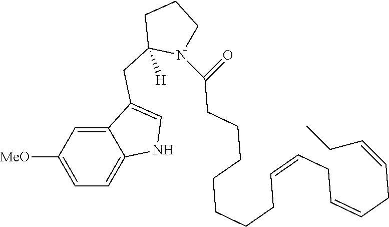 [SMILES: CC/C=C\C/C=C\C/C=C\CCCCCCCC(=O)N1CCC[C@@H]1Cc1c[nH]c2ccc(OC)cc12] |
| (R)-I-73 | 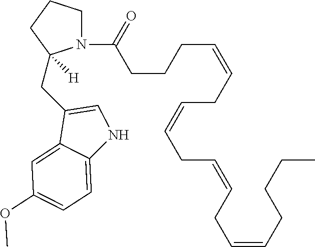 [SMILES: CCCC/C=C\C/C=C\C/C=C\C/C=C\CCCC(=O)N1CCC[C@@H]1Cc1c[nH]c2ccc(OC)cc12] |
| (R)-I-74 | 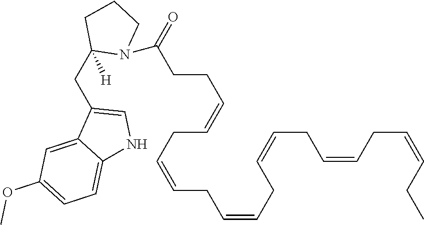 [SMILES: CC/C=C\C/C=C\C/C=C\C/C=C\C/C=C\C/C=C\CCC(=O)N1CCC[C@@H]1Cc1c[nH]c2ccc(OC)cc12] |
| I-75 | 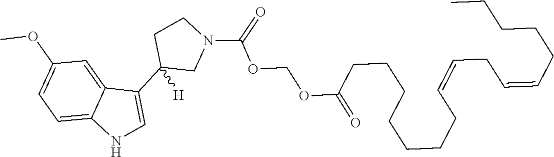 [SMILES: CCCCC/C=C\C/C=C\CCCCCCCC(=O)OCOC(=O)N1CCC(c2c[nH]c3ccc(OC)cc23)C1] |
| (R)-I-76 | 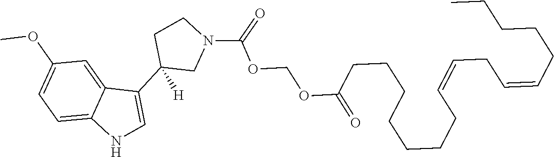 [SMILES: CCCCC/C=C\C/C=C\CCCCCCCC(=O)OCOC(=O)N1CC[C@H](c2c[nH]c3ccc(OC)cc23)C1] |
| (R)-I-77 | 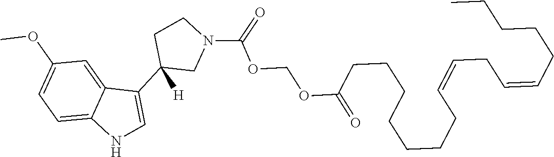 [SMILES: CCCCC/C=C\C/C=C\CCCCCCCC(=O)OCOC(=O)N1CC[C@@H](c2c[nH]c3ccc(OC)cc23)C1] |
| (R)-I-78 | 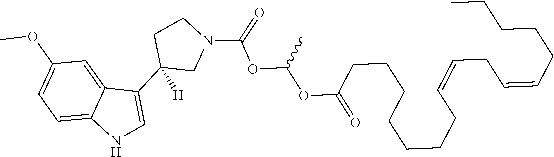 [SMILES: CCCCC/C=C\C/C=C\CCCCCCCC(=O)OC(C)OC(=O)N1CC[C@H](c2c[nH]c3ccc(OC)cc23)C1] |
| (R)-I-79 | 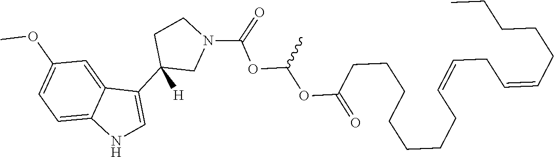 [SMILES: CCCCC/C=C\C/C=C\CCCCCCCC(=O)OC(C)OC(=O)N1CC[C@@H](c2c[nH]c3ccc(OC)cc23)C1] |
| I-80 | 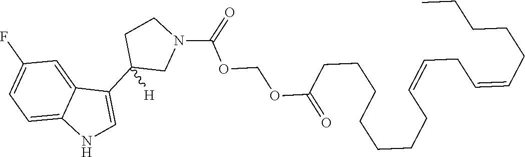 [SMILES: CCCCC/C=C\C/C=C\CCCCCCCC(=O)OCOC(=O)N1CCC(c2c[nH]c3ccc(F)cc23)C1] |
| I-81 | 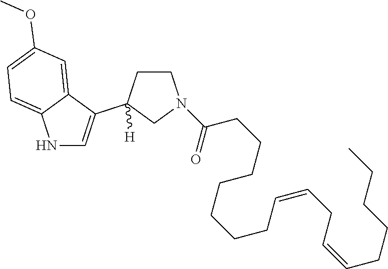 [SMILES: CCCCC/C=C\C/C=C\CCCCCCCC(=O)N1CCC(c2c[nH]c3ccc(OC)cc23)C1] |
| (R)-I-82 | 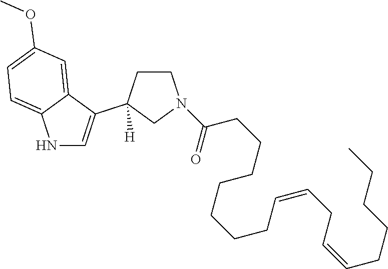 [SMILES: CCCCC/C=C\C/C=C\CCCCCCCC(=O)N1CC[C@H](c2c[nH]c3ccc(OC)cc23)C1] |
| (R)-I-83 | 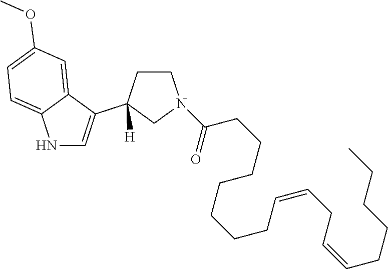 [SMILES: CCCCC/C=C\C/C=C\CCCCCCCC(=O)N1CC[C@@H](c2c[nH]c3ccc(OC)cc23)C1] |
| I-84 | 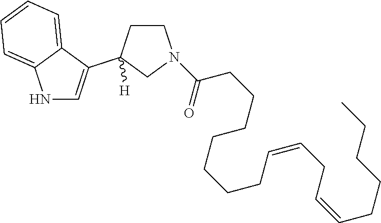 [SMILES: CCCCC/C=C\C/C=C\CCCCCCCC(=O)N1CCC(c2c[nH]c3ccccc23)C1] |
| (R)-I-85 | 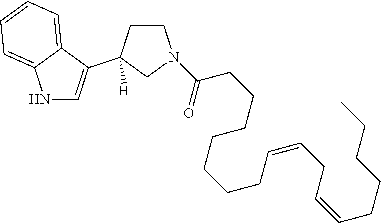 [SMILES: CCCCC/C=C\C/C=C\CCCCCCCC(=O)N1CC[C@H](c2c[nH]c3ccccc23)C1] |
| (R)-I-86 | 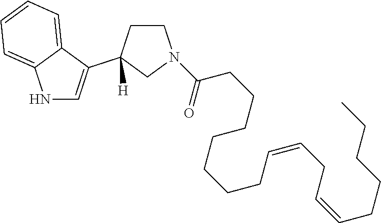 [SMILES: CCCCC/C=C\C/C=C\CCCCCCCC(=O)N1CC[C@@H](c2c[nH]c3ccccc23)C1] |
| I-87 | 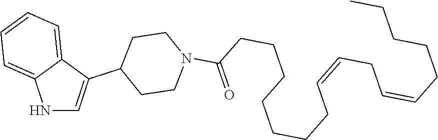 [SMILES: CCCCC/C=C\C/C=C\CCCCCCCC(=O)N1CCC(c2c[nH]c3ccccc23)CC1] |
| I-88 | 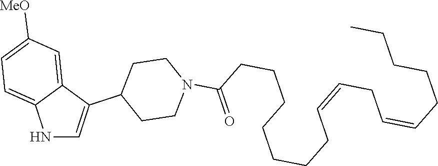 [SMILES: CCCCC/C=C\C/C=C\CCCCCCCC(=O)N1CCC(c2c[nH]c3ccc(OC)cc23)CC1] |
| I-89 | 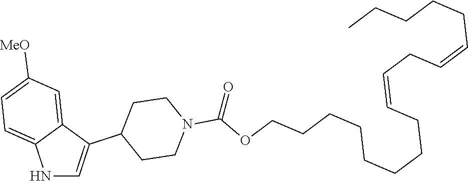 [SMILES: CCCCC/C=C\C/C=C\CCCCCCCCOC(=O)N1CCC(c2c[nH]c3ccc(OC)cc23)CC1] |
| I-90 | 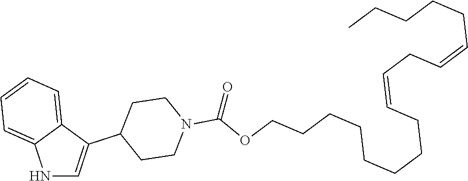 [SMILES: CCCCC/C=C\C/C=C\CCCCCCCCOC(=O)N1CCC(c2c[nH]c3ccccc23)CC1] |
| I-91 | 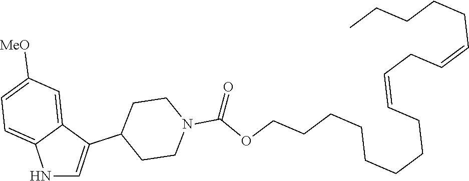 [SMILES: CCCCC/C=C\C/C=C\CCCCCCCCOC(=O)N1CCC(c2c[nH]c3ccc(OC)cc23)CC1] |
| I-92 | 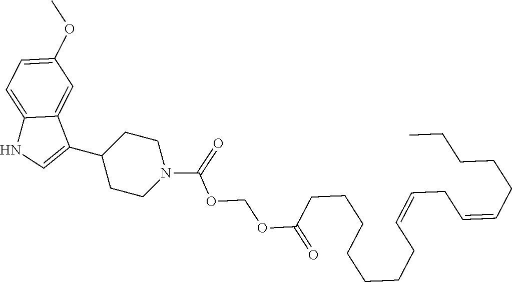 [SMILES: CCCCC/C=C\C/C=C\CCCCCCCC(=O)OCOC(=O)N1CCC(c2c[nH]c3ccc(OC)cc23)CC1] |
| I-93 | 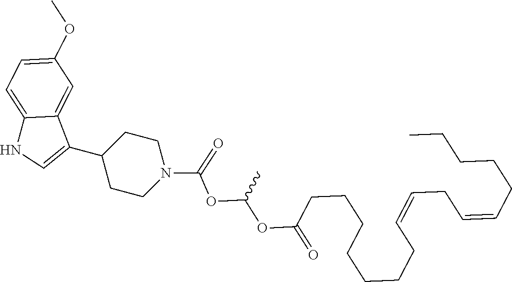 [SMILES: CCCCC/C=C\C/C=C\CCCCCCCC(=O)OC(C)OC(=O)N1CCC(c2c[nH]c3ccc(OC)cc23)CC1] |
| I-94 | 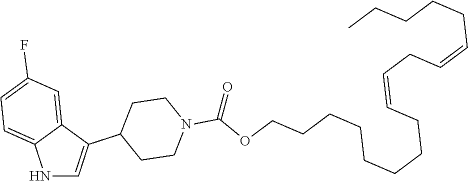 [SMILES: CCCCC/C=C\C/C=C\CCCCCCCCOC(=O)N1CCC(c2c[nH]c3ccc(F)cc23)CC1] |
| I-95 | 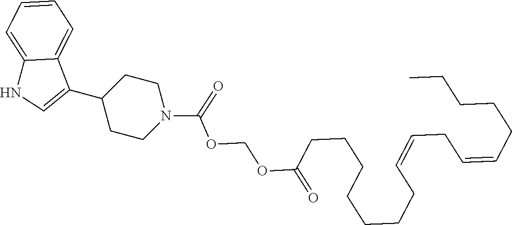 [SMILES: CCCCC/C=C\C/C=C\CCCCCCCC(=O)OCOC(=O)N1CCC(c2c[nH]c3ccccc23)CC1] |
| I-96 | 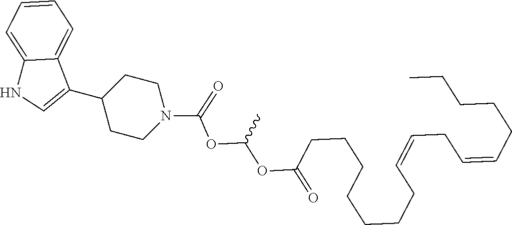 [SMILES: CCCCC/C=C\C/C=C\CCCCCCCC(=O)OC(C)OC(=O)N1CCC(c2c[nH]c3ccccc23)CC1] |
| I-97 | 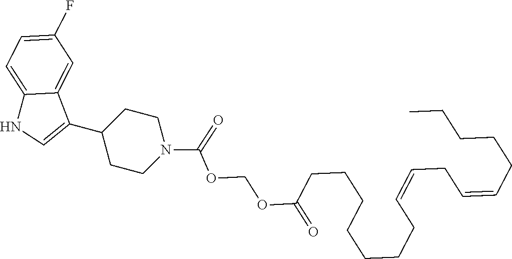 [SMILES: CCCCC/C=C\C/C=C\CCCCCCCC(=O)OCOC(=O)N1CCC(c2c[nH]c3ccc(F)cc23)CC1] |
| I-98 | 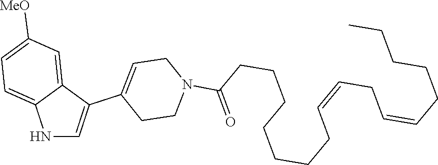 [SMILES: CCCCC/C=C\C/C=C\CCCCCCCC(=O)N1CC=C(c2c[nH]c3ccc(OC)cc23)CC1] |
| (S)-I-99 | 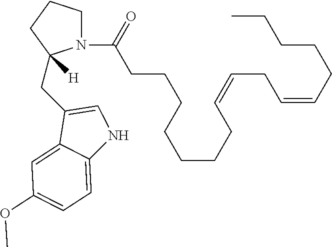 [SMILES: CCCCC/C=C\C/C=C\CCCCCCCC(=O)N1CCC[C@H]1Cc1c[nH]c2ccc(OC)cc12] |
| (S)-I-100 | 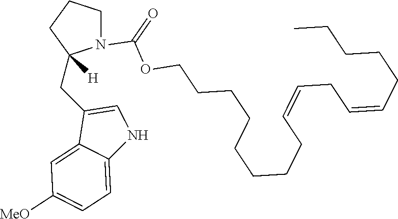 [SMILES: CCCCC/C=C\C/C=C\CCCCCCCCOC(=O)N1CCC[C@H]1Cc1c[nH]c2ccc(OC)cc12] |
| (S)-I-101 | 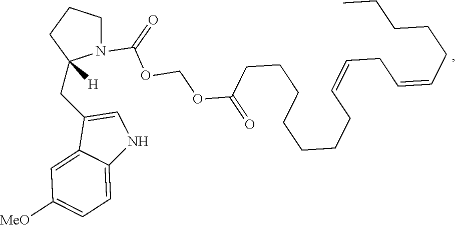 [SMILES: CCCCC/C=C\C/C=C\CCCCCCCC(=O)OCOC(=O)N1CCC[C@H]1Cc1c[nH]c2ccc(OC)cc12] |
| (R)-I-102 | 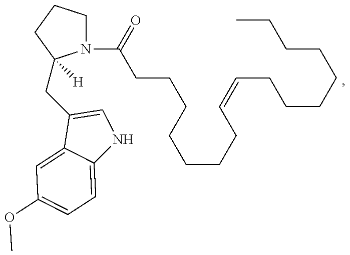 [SMILES: CCCCCCCC/C=C\CCCCCCCC(=O)N1CCC[C@@H]1Cc1c[nH]c2ccc(OC)cc12] |
| | and |
| (S)-I-103 | 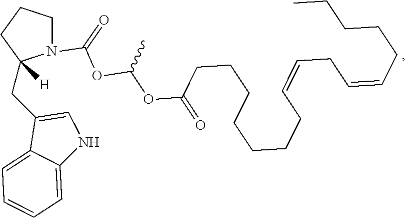 [SMILES: CCCCC/C=C\C/C=C\CCCCCCCC(=O)OC(C)OC(=O)N1CCC[C@H]1Cc1c[nH]c2ccccc12] |

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the present application include, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. Therefore, the compounds of the application also include those in which one or more radioactive atoms are incorporated within their structure.

### III. Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, com starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried com starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application are particularly amenable to administration with the air of nano-carrier systems, such as liposomes, micelles, nanoparticles, nano-emulsions, lipidic nano-systems and the like (see for example, Bhat, M. et al. Chem. and Phys. of Lipids, 2021, 236, 105053). Accordingly the present application includes a composition comprising one or more compounds of the application and one or more components of a nano-carrier system.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient and from about 1 wt % to about 99.95 wt % or about 30 wt % to about 99.90 wt % of a pharmaceutically acceptable carrier, all percentages by weight being based on the total composition.

In some embodiments, the compounds of the application including pharmaceutically acceptable salts and/or solvates thereof are used are administered in a composition comprising an additional therapeutic agent. Therefore the present application also includes a pharmaceutical composition comprising one of more compounds of the application, or pharmaceutically acceptable salts and/or solvates thereof thereof and an additional therapeutic agent, and optionally one or more pharmaceutically acceptable excipients. In some embodiments, the additional therapeutic agent is another known agent useful for treatment of a disease, disorder or condition by activation of a serotonin receptor, for example those listed in the Methods and Uses section below. In some embodiments, the additional therapeutic agent is a psychoactive drug. In some embodiments, the additional therapeutic agent is another known agent useful for treatment of a disease, disorder or condition by modulation of a serotonin receptor, including activating, inhibiting, or antagonizing. In some embodiments, the additional therapeutic agent is a psychoactive drug that modifies release of serotonin, prevents breakdown of serotonin, or activates serotonin receptors. In some embodiments, the additional therapeutic agent is a psychoactive drug that modifies release of serotonin or activates serotonin receptors.

To be clear, in the above, the term “a compound” also includes embodiments wherein one or more compounds are referenced.

### IV. Methods and Uses of the Application

Compounds of the application are useful for treating diseases, disorders or conditions by activating a serotonin receptor. Therefore, the compounds of the present application are useful as medicaments. Accordingly, the application also includes a compound of the application for use as a medicament.

The present application also includes a method of treating a disease, disorder or condition by activation of a serotonin receptor comprising administering a therapeutically effective amount of one or more compounds of the application to a subject in need thereof.

The present application also includes a use of one or more compounds of the application for treatment of a disease, disorder or condition by activation of a serotonin receptor as well as a use of one or more compounds of the application for the preparation of a medicament for treatment of a disease, disorder or condition by activation of a serotonin receptor. The application further includes one or more compounds of the application for use in treating a disease, disorder or condition by activation of a serotonin receptor.

In some embodiments, the serotonin receptor is 5-HT_{2A}. Accordingly, the present application includes a method for activating 5-HT_{2A} in a cell, either in a biological sample or in a patient, comprising administering an effective amount of one or more compounds of the application to the cell. The application also includes a use of one or more compounds of the application for activating 5-HT_{2A} in a cell as well as a use of one or more compounds of the application for the preparation of a medicament for activating 5-HT_{2A} in a cell. The application further includes one or more compounds of the application for use in activating 5-HT_{2A} in a cell.

In some embodiments, the serotonin receptor is 5-HT_{1A}. Accordingly, the present application includes a method for activating 5-HT_{1A} in a cell, either in a biological sample or in a patient, comprising administering an effective amount of one or more compounds of the application to the cell. The application also includes a use of one or more compounds of the application for activating 5-HT_{2A} in a cell as well as a use of one or more compounds of the application for the preparation of a medicament for activating 5-HT_{1A} in a cell. The application further includes one or more compounds of the application for use in activating 5-HT_{1A} in a cell.

The present application also includes a method of treating a disease, disorder or condition by activation of 5-HT_{2A} comprising administering a therapeutically effective amount of one or more compounds of the application to a subject in need thereof. The present application also includes a use of one or more compounds of the application for treatment of a disease, disorder or condition by activation of 5-HT_{2A} as well as a use of one or more compounds of the application for the preparation of a medicament for treatment of a disease, disorder or condition by activation of 5-HT_{2A}. The application further includes one or more compounds of the application for use in treating a disease, disorder or condition by activation of 5-HT_{2A}.

The present application also includes a method of treating a disease, disorder or condition by activation of 5-HT_{1A} comprising administering a therapeutically effective amount of one or more compounds of the application to a subject in need thereof. The present application also includes a use of one or more compounds of the application for treatment of a disease, disorder or condition by activation of 5-HT_{1A} as well as a use of one or more compounds of the application for the preparation of a medicament for treatment of a disease, disorder or condition by activation of 5-HT_{1A}. The application further includes one or more compounds of the application for use in treating a disease, disorder or condition by activation of 5-HT_{1A}.

In some embodiments, the treatment methods and uses of the present application comprise a decreased or lower risk of the subject experiencing or having serotonin syndrome. In some embodiments, the decreased or lower risk is in comparison to a compound corresponding to a compound of the application except that the indole nitrogen is unsubstituted. In some embodiments, the treatment methods and uses comprise any detectable decrease or reduction in the incidences of serotonin syndrome, for example, compared to the incidences of serotonin syndrome after administration or use of a compound corresponding to a compound of the application except that the indole nitrogen is unsubstituted, such as 5-methoxytryptamine or tryptamine.

The disease, disorder or condition may also be treated or treatable via alternative mechanisms, for example by modulation, deactivation, antagonism or reverse agonism of a serotonin receptor, including 5-HT_{2A} and/or 5-HT_{1A}.

In some embodiments, the compounds of the application are useful for preventing, treating and/or reducing the severity of a mental illness disorder and/or condition in a subject. Therefore, in some embodiments, the disease, disorder or condition that is treated by activation of a serotonin receptor is a mental illness. Accordingly, the present application also includes a method of treating a mental illness comprising administering a therapeutically effective amount of one or more compounds of the application to a subject in need thereof. The present application also includes a use of one or more compounds of the application for treatment a mental illness, as well as a use of one or more compounds of the application for the preparation of a medicament for treatment of a mental illness. The application further includes one or more compounds of the application for use in treating a mental illness.

In some embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, schizophrenia; impulse control and addiction disorders, such as pyromania (starting fires), kleptomania (stealing) and compulsive gambling; alcohol addiction; drug addiction, such as opioid addiction; personality disorders, such as antisocial personality disorder, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilia's; somatic symptom disorders, formerly known as a psychosomatic disorder or somatoform disorder; and combinations thereof.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the mental illness is selected from hallucinations and delusions and a combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering a therapeutically effective amount of one or more compounds of the application to a subject in need thereof.

The present application also includes a use of one or more compounds of the application for treatment of psychosis or psychotic symptoms, as well as a use of one or more compounds of the application for the preparation of a medicament for treatment of psychosis or psychotic symptoms. The application further includes one or more compounds of the application for use in treating psychosis or psychotic symptoms.

In some embodiments, administering to said subject in need thereof a therapeutically effective amount of the compounds of the application does not result in a worsening of psychosis or psychotic symptoms such as, but not limited to, hallucinations and delusions. In some embodiments, administering to said subject in need thereof a therapeutically effective amount of the compounds of the application results in an improvement of psychosis or psychotic symptoms such as, but not limited to, hallucinations and delusions. In some embodiments, administering to said subject in need thereof a therapeutically effective amount of the compounds of the application results in an improvement of psychosis or psychotic symptoms.

In some embodiments, the compounds of the application are useful for treating a central nervous system (CNS) disorder in a subject in need of therapy, comprising administering a therapeutically effective amount of a compound of general formula (I), or a pharmaceutically acceptable salt thereof to the subject.

Therefore, in some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition comprising administering a therapeutically effective amount of one or more compounds of the application to a subject in need thereof. The present application also includes a use of one or more compounds of the application for treatment a CNS disease, disorder or condition and/or a neurological disease, disorder or condition, as well as a use of one or more compounds of the application for the preparation of a medicament for treatment of a CNS disease, disorder or condition and/or a neurological disease, disorder or condition. The application further includes one or more compounds of the application for use in treating a CNS disease, disorder or condition and/or a neurological disease, disorder or condition. In some embodiments the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

The present application also includes a method of treating a neurological disease, disorder or condition comprising administering a therapeutically effective amount of one or more compounds of the application to a subject in need thereof, as well as a use of one or more compounds of the application to treat a neurological disease, disorder or condition, a use of one or more compounds of the application to prepare a medicament to treat a neurological disease, disorder or condition and one or more compounds of the application for use to treat a neurological disease, disorder or condition.

In some embodiments, the subject is a mammal. In another embodiment, the subject is human. In some embodiments, the subject is a non-human animal. In some embodiments, the subject is canine. In some embodiments, the subject is feline. Accordingly, the compounds, methods and uses of the present application are directed to both human and veterinary diseases, disorders and conditions.

In some embodiments, the compounds of the application are useful for treating behavioral problems in subjects that are felines or canines.

Therefore, in some embodiments, the disease, disorder or condition that is treated by activation of a serotonin receptor is behavioral problems in subjects that are felines or canines. Accordingly, the present application also includes a method of treating a behavioral problem comprising administering a therapeutically effective amount of one or more compounds of the application to a non-human subject in need thereof. The present application also includes a use of one or more compounds of the application for treatment a behavioral problem in a non-human subject, as well as a use of one or more compounds of the application for the preparation of a medicament for treatment of a behavioral problem in a non-human subject. The application further includes one or more compounds of the application for use in treating a behavioral problem in a non-human subject.

In some embodiments, the behavioral problems are selected from, but are not limited to, anxiety, fear, stress, sleep disturbances, cognitive dysfunction, aggression, excessive noise making, scratching, biting and a combination thereof.

In some embodiments, the non-human subject is canine. In some embodiments, the non-human subject is feline.

The present application also includes a method of treating a disease, disorder or condition by activation of a serotonin receptor comprising administering a therapeutically effective amount of one or more compounds of the application in combination with another known agent useful for treatment of a disease, disorder or condition by activation of a serotonin receptor to a subject in need thereof. The present application also includes a use of one or more compounds of the application in combination with another known agent useful for treatment of a disease, disorder or condition by activation of a serotonin receptor for treatment of a disease, disorder or condition by activation of a serotonin receptor, as well as a use of one or more compounds of the application in combination with another known agent useful for treatment of a disease, disorder or condition by activation of a serotonin receptor for the preparation of a medicament for treatment of a disease, disorder or condition by activation of a serotonin receptor. The application further includes one or more compounds of the application in combination with another known agent useful for treatment of a disease, disorder or condition by activation of a serotonin receptor for use in treating a disease, disorder or condition by activation of a serotonin receptor.

In some embodiments, the disease, disorder or condition that is treated by activation of a serotonin receptor is a mental illness. In some embodiments, the mental illness is selected from hallucinations and delusions and a combination thereof. In some embodiments, the disease, disorder or condition that is treated by activation of a serotonin receptor is a central nervous system (CNS) disorder. In some embodiments, the disease, disorder or condition that is treated by activation of a serotonin receptor is psychosis or psychotic symptoms. In some embodiments, the disease, disorder or condition that is treated by activation of a serotonin receptor is behavioral problems in a non-human subject.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; mood stabilizers such as lithium and anticonvulsants such carbamazepine, divalproex (valproic acid), lamotrigine, gabapentin and topiramate.

In some embodiments, the disease, disorder or condition that is treated by activation of a serotonin receptor is selected from attention deficit hyperactivity disorder and attention deficit disorder and a combination thereof. In some embodiments, the disease, disorder or condition that is treated by activation of a serotonin receptor is attention deficit hyperactivity disorder and/or attention deficit disorder and a combination thereof and the one or more compounds of the application are administered in combination with one or more additional treatments for attention deficit hyperactivity disorder and/or attention deficit disorder and a combination thereof. In some embodiments, the additional treatments for attention deficit hyperactivity disorder and/or attention deficit disorder and a combination thereof are selected from methylphenidate, atomoxetine and amphetamine and a combination thereof.

In some embodiments, the disease, disorder or condition that is treated by activation of a serotonin receptor is dementia or Alzheimer's disease and the one or more compounds of the application are administered in combination with one or more additional treatments for dementia or Alzheimer's disease. In some embodiments, the additional treatments for dementia and Alzheimer's disease are selected acetylcholinesterase inhibitors, NMDA antagonists and muscarinic agonists and antagonists, and nicotinic agonists.

In some embodiments, the acetylcholinesterase inhibitors are selected from donepezil, galantamine, rivastigmine, and phenserine, and combinations thereof.

In some embodiments, the NMDA antagonists are selected from MK-801, ketamine, phencyclidine, and memantine, and combinations thereof.

In some embodiments, the nicotinic agonists is nicotine, nicotinic acid, nicotinic alpha7 agonists or alpha2 beta4 agonists or combinations thereof.

In some embodiments, the muscarinic agonists is a muscarinic M1 agonist or a muscarinic M4 agonist, or combinations thereof.

In some embodiments, the muscarinic antagonist is a muscarinic M2 antagonist.

In some embodiments, the disease, disorder or condition that is treated by activation of a serotonin receptor is psychosis or psychotic symptoms and the one or more compounds of the application are administered in combination with one or more additional treatments for psychosis or psychotic symptoms. In some embodiments, the additional treatments for psychosis or psychotic symptom are selected typical antipsychotics and atypical antipsychotics.

In some embodiments, the typical antipsychotics are selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, Ioxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof.

In some embodiments, the atypical antipsychotics are selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected typical antipsychotics and atypical antipsychotics.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiment, the compounds of the application are administered one, two, three or four times a year. In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of the application is either used alone or in combination with other known agents useful for treating diseases, disorders or conditions by activation of a serotonin receptor, such as the compounds of the application. When used in combination with other known agents useful in treating diseases, disorders by activation of a serotonin receptor, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. One of skill in the art can determine the appropriate dosage based on the above factors. In some embodiments, one or more compounds of the application are administered initially in a suitable dosage that is adjusted as required, depending on the clinical response. Dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of the application will range between about 10 μg per day to about 1000 mg per day for an adult, suitably about 10 μg per day to about 500 mg per day, more suitably about 10 μg per day to about 200 mg per day. For parenteral administration, a representative amount is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered. For oral administration, a representative amount is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative amount is from about 0.1 mg/kg to about 10 mg/kg or about 0.1 mg/kg to about 1 mg/kg. In some embodiments of the application, compositions are formulated for oral administration and the one or more compounds are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient (one or more compounds of the application) per tablet. In some embodiments of the application the one or more compounds of the application are administered in a single daily, weekly or monthly dose or the total daily dose is divided into two, three or four daily doses.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

To be clear, in the above, the term “a compound” also includes embodiments wherein one or more compounds are referenced. Likewise, the term “compounds of the application” also includes embodiments wherein only one compound is referenced.

### Preparation of Compounds

Compounds of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources or may be extracted from cells, plants, animals or fungi. Other starting materials, for example as described below, are readily prepared from available precursors using straightforward transformations that are well known in the art. In the Schemes below showing some embodiments of methods of preparation of compounds of the application, all variables are as defined in Formula I, unless otherwise stated.

In some embodiments, when Q is

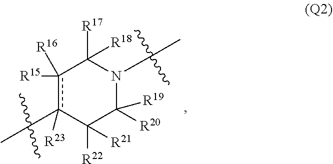

[symbol]

is a single bond, and R¹⁵—R²³ are as defined in Formula I, the compounds of Formula I are prepared as shown In Scheme 1:

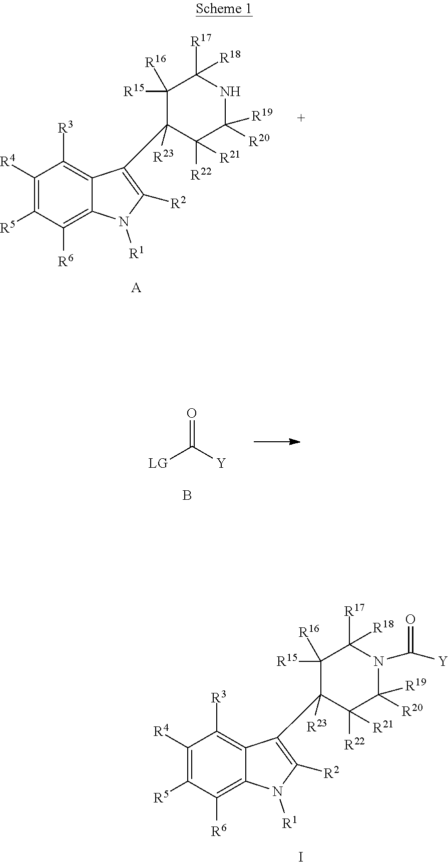

Therefore, in some embodiments, compounds of formula A, wherein R¹—R⁶ are as defined in formula I, are reacted under basic conditions using for example, triethyl amine with compounds of formula B, wherein Y is as defined in Formula I and LG is a suitable leaving group, such as chloro, to provide compounds of formula I. A person skilled in the art would appreciate that a similar reaction sequence can be used to prepare compounds of formula I, wherein Q is

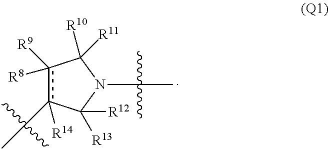

In some embodiments, when Q is

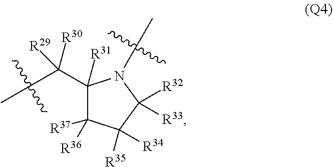

R²⁹ and R³⁰ are either H or D and R³¹—R³⁷ are as defined in Formula I, the compounds of Formula I are prepared as shown In Scheme 2:

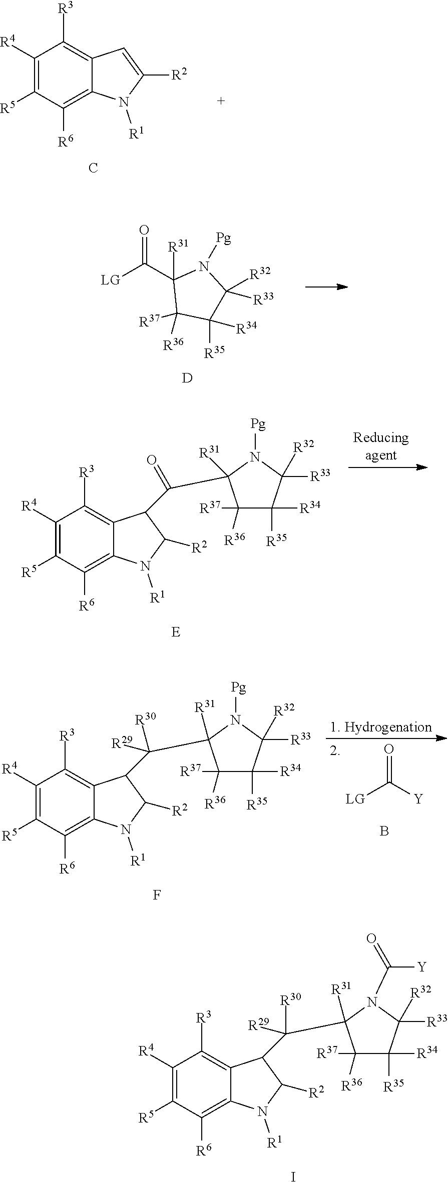

Therefore, in some embodiments, compounds of Formula C, wherein R¹—R⁶ are as defined in Formula I, are reacted under basic conditions using for example, ethyl magnesium bromide with compounds of formula D, wherein R³¹—R³⁷ are as defined in Formula I and LG is a suitable leaving group, such as chloro to form compounds of formula E, wherein Pg is a suitable protecting group such as benzyl group. Keto group is reduced using for example, LiBH₄ LiBH₄ or LiAlD₄ to form compounds of formula F, wherein R²⁹ and R³⁰ are as defined in formula I. Then compounds of formula F are hydrogenated in the presence of a palladium catalyst, and are reacted with compound of formula B, wherein Y is as defined in compound of Formula I and LG is a suitable leaving group, such as chloro, to form compounds of formula I. A person skilled in the art would appreciate that a similar reaction sequence can be used to prepare compounds of formula I, wherein Q is

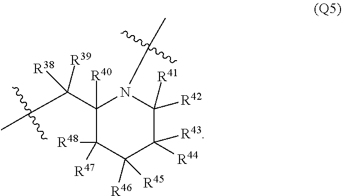

In some embodiments, when Q is

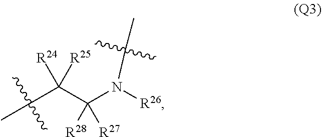

R²⁴, R²⁵, R²⁷ and R²⁸ are either H or D, and R²⁶ is as defined in formula I, the compounds of formula I are prepared as shown in Scheme 3:

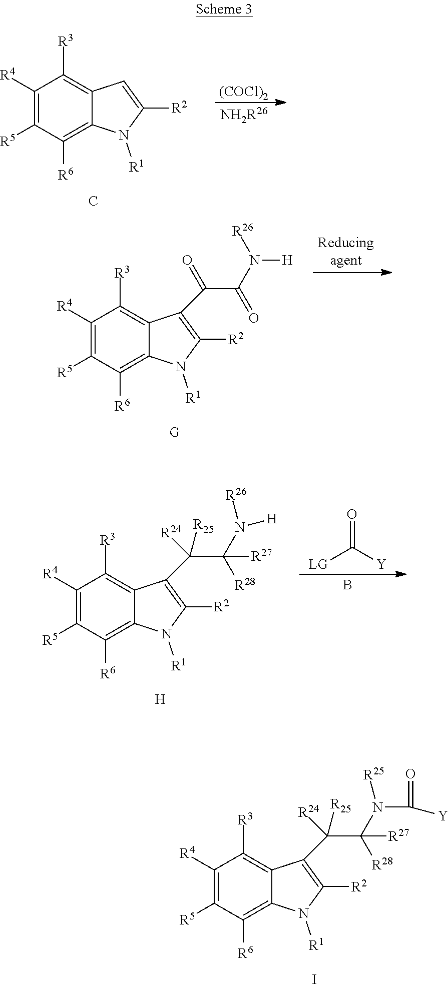

Therefore, in some embodiments, compounds of formula C, wherein R¹—R⁶ are as defined in Formula I, are reacted with oxalyl chloride followed by an amine NH₂R²⁶, wherein R²⁶ are as defined in Formula I, to provide compounds of formula G. Then compound G is reduced using for example LiAlH₄, LiBH₄ or LiAlD₄ to form compound H, and is reacted with compound of formula B, in which LG is a suitable leaving group such as chloro and Y is as defined in Formula I, to form compound of formula I.

In some embodiments, when Q is

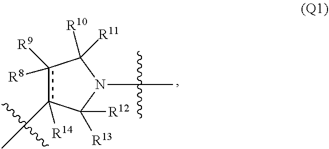

[symbol]

is a single bond, R¹⁰—R¹³ are either H or D and R⁸, R⁹ and R¹⁴ are as defined in Formula I, the compounds of Formula I are prepared as shown in Scheme 4:

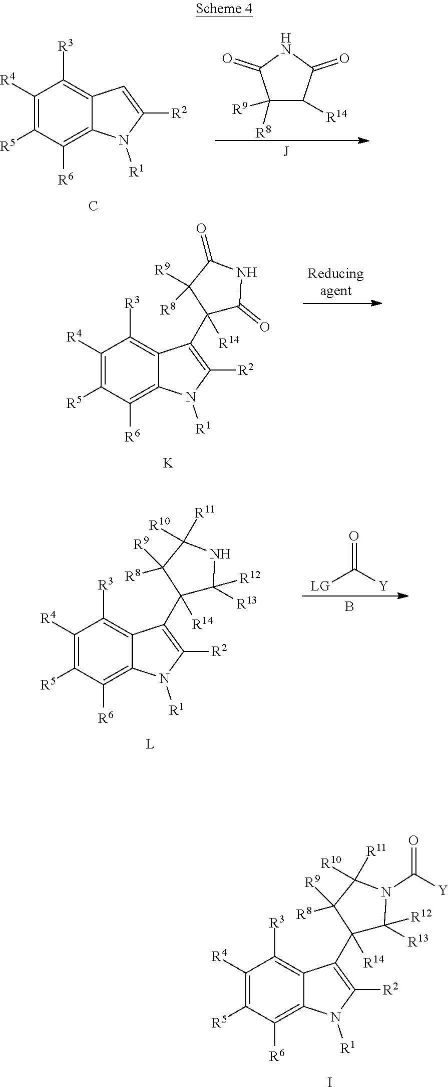

Therefore, in some embodiments, compounds of formula C, wherein R¹—R⁶ are as defined in Formula I, are coupled with compounds of Formula J, wherein R⁸, R⁹ and R¹⁴ are as defined in Formula I, in a suitable solvent such as acetic acid, to provide compounds of Formula K. Keto group is reduced using for example LiAlH₄, LiBH₄ or LiAlD₄ to form compounds of formula L, wherein R¹⁰—R¹³ are either H or D. The resulting compounds of formula L is then reacts with compounds of formula B, wherein Y is as defined in Formula I and LG is a suitable leaving group such as chloro to provide compounds of formula I. A person skilled in the art would appreciate that a similar reaction sequence can be used to prepare compounds of Formula A, wherein Q is

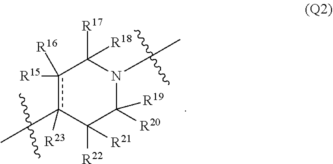

Compounds of Formula I, wherein one or more of R², R³—R⁶ and/or one or more of R²³—R²⁷ are deuterium are available, for example, using a hydrogen-deuterium exchange reaction on a suitable starting substrate, wherein this exchange reaction is catalyzed by Pd/C in D₂O as described in Esaki, H. et al. Tetrahedron, 2006, 62:10954-10961, and modifications thereof known to a person skilled in the art.

Compounds of Formula I wherein R⁴ is OCD₃ are available, for example, using methods as described in Xu, Y—Z and Chen, C. J. Label Compd. Radiopharm. (2006) 49:897-902, and modifications thereof and modifications thereof known to a person skilled in the art.

Compounds of Formula B, C, D and J as well as other reagents used in the above Schemes, are available from commercial sources or can be readily prepared using methods known in the art.

A person skilled in the art would appreciate that further manipulation of the substituent groups using known chemistry can be performed on the intermediates and final compounds in the Schemes above to provide alternative compounds of the application.

Salts of compounds of the application may be formed by methods known to those of ordinary skill in the art, for example, by reacting a compound of the application with an amount of acid or base, such as an equivalent amount, in a medium such as one in which the salt precipitates or in aqueous medium followed by lyophilization.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

Isotopically-enriched compounds of the application and pharmaceutically acceptable salts and/or solvates thereof, can be prepared without undue experimentation by conventional techniques well known to those skilled in the art or by processes analogous to those described in the Schemes and Examples herein using suitable isotopically-enriched reagents and/or intermediates.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

### Examples

### A. Chemical Synthesis

### Example 1: (9Z,12Z)-1-(4-(5-Methoxy-1H-indol-3-yl)piperidin-1-yl)octadeca-9,12-dien-1-one (I-88)

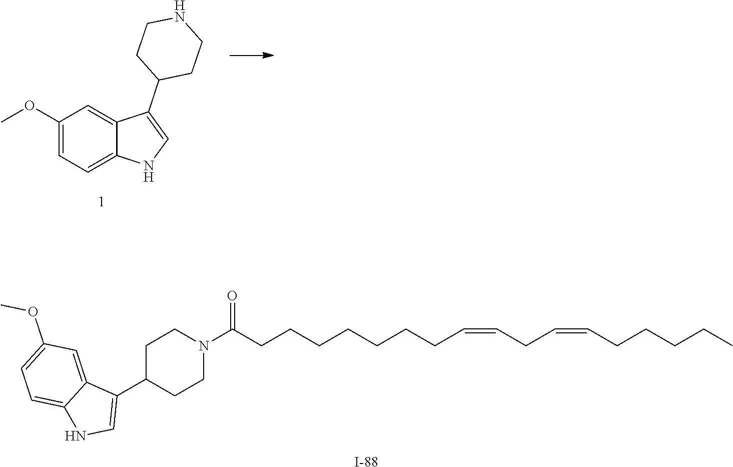

### Synthesis of (9Z,12Z)-1-(4-(5-methoxy-1H-indol-3-yl)piperidin-1-yl)octadeca-9,12-dien-1-one (1-88)

A solution of linoleic acid (3.65 g, 13.02 mmol) in dry CH₂Cl₂ (20 mL) was treated with oxalyl chloride (1.47 mL, 17.36 mmol) followed by catalytic DMF (1 drop) at room temperature and stirred for additional 2 h. Solvent was evaporated to obtain crude acid chloride as a brown oil.

A solution of 5-methoxy-3-(piperidin-4-yl)-1H-indole (1.0 g, 4.34 mmol) in dry DMF (15 mL) was treated with Et₃N (2.42 mL, 17.36 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-88 (1.8 g, 84%) as an off-white semi-solid. ¹H NMR (CDCl₃): δ 7.95 (s, 1H), 7.29 (d, 1H, J=6.0 Hz), 7.07 (d, 1H, J=3.0 Hz), 6.96 (s, 1H), 6.90 (dd, 1H, J=3.0, 6.0 Hz), 5.44-5.32 (m, 4H), 4.81 (d, 1H, J=9.0 Hz), 4.02-3.98 (m, 1H), 3.98 (s, 3H), 3.28-3.24 (m, 1H), 3.10-3.02 (m, 1H), 2.81-2.73 (m, 3H), 2.40)t, 2H, J=6.0 Hz), 2.18-2.05 (m, 6H), 1.74-1.60 (m, 5H), 1.42-1.28 (m, 13H), 0.90 (t, 3H, J=6.0 Hz); ESI-MS (m/z, %): 515 (M+Na, 100), 493 (MM+).

### Example 2: (9Z,12Z)-1-(4-(1H-Indol-3-yl)piperidin-1-yl)octadeca-9,12-dien-1-one (1-87)

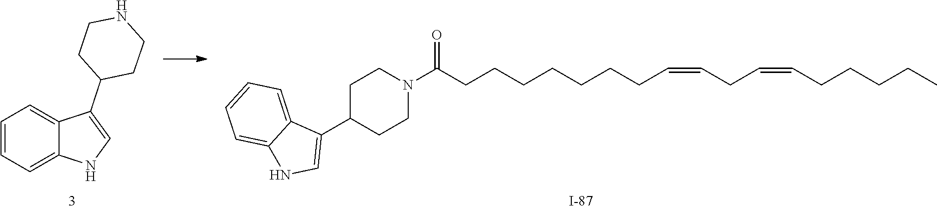

### Synthesis of (9Z,12Z)-1-(4-(1H-indol-3-yl)piperidin-1-yl)octadeca-9,12-dien-1-one (I-80)

A solution of linoleic acid (4.20 g, 14.98 mmol) in dry CH₂Cl₂ (20 mL) was treated with oxalyl chloride (1.69 mL, 19.97 mmol) followed by catalytic DMF (1 drop) at room temperature and stirred for additional 2 h. Solvent was evaporated to obtain crude acid chloride as a brown oil.

A solution of 3-(piperidin-4-yl)-1H-indole (1.0 g, 4.99 mmol) in dry DMF (15 mL) was treated with Et₃N (2.78 mL, 19.97 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-87 (2.17 g, 94%) as a light brown glue. ¹H NMR (CDCl₃): δ 8.09 (s, 1H), 7.65 (d, 1H, J=6.0 Hz), 7.40 (d, 1H, J=6.0 Hz), 7.25-7.20 (m, 1H), 7.16-7.12 (m, 1H), 6.98 (s, 1H), 5.42-5.34 (m, 4H), 4.83-4.79 (m, 1H), 4.01-3.98 (m, 1H), 3.24-3.20 (m, 1H), 3.11-3.01 (m, 1H), 2.82-2.76 (m, 3H), 2.40 (t, 2H, J=6.0 Hz), 2.19-2.05 (m, 6H), 1.74-1.63 (m, 5H), 1.42-1.28 (m, 13H), 0.92 (t, 3H, J=3.0 Hz); ESI-MS (m/z, %): 485 (M+Na, 100), 463 (MH⁺).

### Example 3: (9Z,12Z)-1-((R)-2-((5-Methoxy-1H-indol-3-yl)methyl)pyrrolidin-1-yl)octadeca-9,12-dien-1-one (1-55)

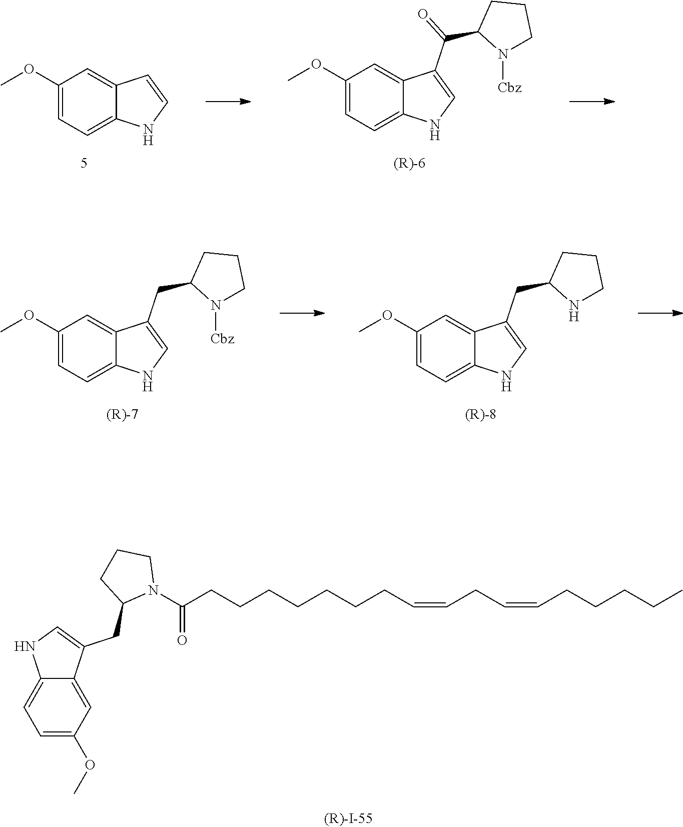 [SMILES: COc1ccc2[nH]ccc2c1 ; COc1ccc2[nH]cc(C(=O)[C@H]3CCCN3C(=O)OCc3ccccc3)c2c1 ; COc1ccc2[nH]cc(C[C@H]3CCCN3C(=O)OCc3ccccc3)c2c1 ; COc1ccc2[nH]cc(C[C@H]3CCCN3)c2c1 ; CCCCC/C=C\C/C=C\CCCCCCCC(=O)N1CCC[C@@H]1Cc1c[nH]c2ccc(OC)cc12]

### Synthesis of (R)-2-(2-(5-methoxy-1H-indole-3-carbonyl)pyrrolidin-1-yl)-1-phenyl-2λ²-ethan-1-one ((R)-6)

A solution of (2-oxo-2-phenyl-1λ²-ethyl)-D-proline (17.4 g, 69.806 mmol) in dry THF (100 mL) was treated with thionyl chloride (10.2 mL, 139.613 mmol) at 0° C. The reaction was brought to room temperature, then refluxed for 2 h. The reaction was brought to room temperature, solvent was evaporated and crude was dried under vacuum to obtain the corresponding acid chloride.

A solution of 5-methoxy-1H-indole (10.2 g, 69.806 mmol) in dry CH₂Cl₂ (100 mL) at 5-10° C. was treated with above crude acid chloride in dry CH₂Cl₂ (40 mL) and ethyl magnesium bromide (46.5 mL, 139.61 mmol, 3 M in THF) simultaneously over a period of 15 min. and stirred at same temperature for further 15 min. The reaction was quenched with con. HCl (10 mL) followed by water (150 mL) and product was extracted into CH₂Cl₂ (2×150 mL). CH₂Cl₂ layer was washed with sat. NaHCO₃ solution (50 mL), brine (25 mL) and dried (Na₂SO₄). Solvent was evaporated and crude was purified by flash column chromatography (CH₂Cl₂ to EtOAc:CH₂Cl₂, 1:4) on silica gel to obtain the title compound (R)-6 (18.9 g, 71.5%) as a yellow solid.; ESI-MS (m/z, %): 401 (M+Na, 100), 379 (MH⁺).

### Synthesis of (R)-2-(2-((5-methoxy-1H-indol-3-yl)methyl)pyrrolidin-1-yl)-1-phenyl-2λ²-ethan-1-one ((R)-7)

A solution of (R)-2-(2-(5-methoxy-1H-indole-3-carbonyl)pyrrolidin-1-yl)-1-phenyl-2λ²-ethan-1-one (1.6 g, 4.227 mmol) in dry THF (25 mL) was treated with a solution of lithium borohydride (8.45 mL, 16.911 mmol, 2 M solution in THF) at room temperature over a period of 5 min. and the reaction was refluxed for additional 4 hours. The reaction was cooled to 0° C., quenched with the careful addition of methanol (10 mL) over a period of 15 min. The reaction was brought to room temperature and stirred for additional 1 h. The reaction was treated with sat. NaHCO₃ solution (30 mL) and product was extracted into ethyl acetate (3×50 mL). Combined ethyl acetate layer was washed with brine (20 mL) and dried (Na₂SO₄). Solvent was evaporated and crude was purified by flash column chromatography (MeOH; CH₂Cl₂, 2:98) on silica gel to obtain the title compound (R)-7 (1.17 g, 76%) as a pale yellow semi-solid. ¹H NMR (CDCl₃): δ 7.91 (s, 1H), 7.46-7.18 (m, 6H), 7.05-6.85 (m, 3H), 5.27-5.20 (m, 2H), 3.70 (S, 3H), 3.51-3.44 (m, 2H), 3.48-3.44 (m, 1H), 3.35-3.30 (m, 1H), 2.82-2.71 (m, 1H), 1.70-1.60 (m, 4H); ESI-MS (m/z, %): 387 (M+Na, 100).

### Synthesis of (R)-5-methoxy-3-(pyrrolidin-2-ylmethyl)-1H-indole ((R)-8)

A suspension of (R)-2-(2-((5-methoxy-1H-indol-3-yl)methyl)pyrrolidin-1-yl)-1-phenyl-2λ2-ethan-1-one (3.0 g, 8.23 mmol) in methanol (50 mL) was treated with 10% palladium on carbon (3.0 g) and hydrogenated using Parr apparatus at 50 PSI for 20 h. The reaction was filtered through a pad celite and washed with methanol (3×25 mL). Combined methanol layer was evaporated and crude was purified by flash column chromatography (2 M NH₃ in MeOH:CH₂Cl₂, 1:9) on silica gel to obtain the title compound (R)-8 (1.23 g, 64.7%) as a beige foam. ¹H NMR (DMSO-d₆): δ 10.64 (s, 1H), 7.22 (s, 1H, J=6.0 Hz), 7.12 (s, 1H), 7.00 (s, 1H), 6.71 (dd, 1H, J=3.0, 7.5 Hz), 3.76 (s, 3H), 3.32-3.25 (m, 1H), 2.97-2.91 (m, 1H), 2.82-2.67 (m, 3H), 1.79-1.58 (m, 3H), 1.40-1.30 (m, 1H); ESI-MS (m/z, %): 231 (MH⁺, 100).

### Synthesis of (9Z,12Z)-1-((R)-2-((5-Methoxy-1H-indol-3-yl)methyl)pyrrolidin-1-yl)octadeca-9,12-dien-1-one ((R)—I-55)

A solution of linoleic acid (1.82 g, 6.51 mmol) in dry CH₂Cl₂ (20 mL) was treated with oxalyl chloride (0.73 mL, 8.68 mmol) followed by catalytic DMF (1 drop) at room temperature and stirred for additional 2 h. Solvent was evaporated to obtain crude acid chloride as a brown oil.

A solution of (R)-5-methoxy-3-(pyrrolidin-2-ylmethyl)-1H-indole (0.5 g, 2.17 mmol) in dry DMF (15 mL) was treated with Et₃N (1.21 mL, 8.68 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound (R)—I-55 (0.82 g, 76.6%) as a yellow glue. ¹H NMR (CDCl₃): δ 8.03, 7.96 (2s, 1H), 7.33-7.24 (m, 2H), 7.03-6.99 (m, 1H), 6.88-6.85 (m, 1H), 5.42-5.34 (m, 4H), 4.55-5.45, 4.25-4.40 (2m, 1H), 3.91, 3.89 (2s, 3H), 3.60-3.32 (m, 3H), 2.82-2.65 (M, 3H), 2.34-2.30 (m, 2H), 2.10-2.05 (m, 4H), 1.94-1.72 (m, 6H), 1.42-1.27 (m, 14H), 0.93-0.89 (m, 3H); ESI-MS (m/z, %): 515 (M+Na), 493 (MH⁺).

### Example 4: (9Z,12Z)-1-((R)-2-((1H-Indol-3-yl)methyl)pyrrolidin-1-yl)octadeca-9,12-dien-1-one (1-46)

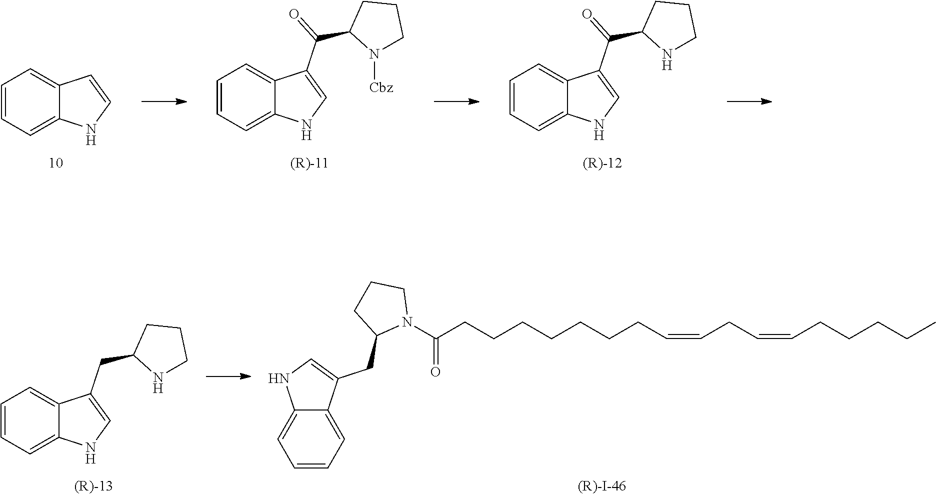 [SMILES: c1ccc2[nH]ccc2c1 ; O=C(c1c[nH]c2ccccc12)[C@H]1CCCN1C(=O)OCc1ccccc1 ; O=C(c1c[nH]c2ccccc12)[C@H]1CCCN1 ; c1ccc2c(C[C@H]3CCCN3)c[nH]c2c1 ; CCCCC/C=C\C/C=C\CCCCCCCC(=O)N1CCC[C@@H]1Cc1c[nH]c2ccccc12]

### Synthesis of (R)-2-(2-(1H-indole-3-carbonyl)pyrrolidin-1-yl)-1-phenyl-2λ²-ethan-1-one ((R)-11)

A solution of (2-oxo-2-phenyl-1λ²-ethyl)-D-proline (15.4 g, 61.782 mmol) in dry THF (70 mL) was treated with thionyl chloride (9.0 mL, 123.56 mmol) at 0° C. The reaction was brought to room temperature, then refluxed for 2 h. The reaction was brought to room temperature, solvent was evaporated and crude was dried under vacuum to obtain the corresponding acid chloride.

A solution of 1H-indole (7.23 g, 61.782 mmol) in dry CH₂Cl₂ (50 mL) at 5-10° C. was treated with above crude acid chloride in dry CH₂Cl₂ (50 mL) and ethyl magnesium bromide (41.2 mL, 123.56 mmol, 3 M in THF) simultaneously over a period of 15 min. and stirred at same temperature for further 15 min. The reaction was quenched with con. HCl (10 mL) followed by water (150 mL) and product was extracted into CH₂Cl₂ (2×150 mL). CH₂Cl₂ layer was washed with sat. NaHCO₃ solution (50 mL), brine (25 mL) and dried (Na₂SO₄). Solvent was evaporated and crude was purified by flash column chromatography (CH₂Cl₂ to EtOAc:CH₂Cl₂, 1:4) on silica gel to obtain the title compound (R)-11 (13.0 g, 60.4%) as a light brown solid. ¹H NMR (CDCl₃): δ 9.54, 9.24 (2s, 1H), 8.43, 8.20 (2d, 1H, J=3.0 Hz), 7.70-7.64 (m, 1H), 7.44-7.00 (m, 8H), 5.29-5.01 (m, 3H), 3.81-3.63 (m, 2H), 2.28-2.25 (m, 1H), 2.09-1.94 (m, 3H); ESI-MS (m/z, %): 371 (M+Na, 100), 349 (MH⁺).

### Synthesis of (R)-3-prolyl-1H-indole ((R)-12)

A suspension of (R)-2-(2-(1H-indole-3-carbonyl)pyrrolidin-1-yl)-1-phenyl-2λ²-ethan-1-one (1.2 g, 3.444 mmol) in methanol (25 mL) was treated with 10% palladium on carbon (1.2 g) and hydrogenated using Parr apparatus at 50 PSI for 50 min. The reaction was filtered through a pad celite and washed with methanol (3×25 mL). Combined methanol layer was evaporated and crude was purified by crystallization from a mixture of CH₂Cl₂:hexanes (1:1) to obtain the title compound (R)-12 (0.48 g, 65%) as an off-white solid. ¹H NMR (DMSO-d₆): δ 8.41 (d, 1H, J=3.0 Hz), 8.21 (dd, 1H, J=3.0 Hz), 7.50-7.46 (m, 1H), 7.25-7.18 (m, 2H), 4.42-4.39 (m, 1H), 3.10-3.04 (m, 1H), 2.79-2.75 (m, 1H), 2.20-2.18 (m, 1H), 1.76-1.62 (m, 3); ESI-MS (m/z, %): 215 (MH⁺, 100).

### Synthesis of (R)-3-(pyrrolidin-2-ylmethyl)-1H-indole ((R)-13)

A solution of (R)-3-prolyl-1H-indole (2.0 g, 9.33 mmol) in dry THF (40 mL) was treated with a solution of lithium borohydride (18.6 mL, 37.33 mmol, 2 M solution in THF) at room temperature over a period of 5 min. and the reaction was refluxed for additional 4 hours. The reaction was cooled to 0° C., quenched with the careful addition of methanol (25 mL) over a period of 15 min. The reaction was brought to room temperature and stirred for additional 1 h. The reaction was treated with sat. NaHCO₃ solution (30 mL) and product was extracted into ethyl acetate (3×50 mL). Combined ethyl acetate layer was washed with brine (20 mL) and dried (Na₂SO₄). Solvent was evaporated and crude was purified by flash column chromatography (MeOH; CH₂Cl₂, 1:9) on silica gel to obtain the title compound (R)-13 (0.63 g, 33.7%) as an off-white solid. ¹H NMR (DMSO-d₆): δ 10.83 (s, 1H), 7.54 (d, 1H, J=6.0 Hz), 7.34 (d, 1H, J=6.0 Hz), 7.18 (d, 1H, J=3.0 Hz), 7.08-7.04 (m, 1H), 6-99-6.95 (m, 1H), 3.37-3.34 (m, 1H), 2.99-2.75 (m, 4H), 1.80-1.63 (m, 3H), 1.42-1.37 (m, 1H); ESI-MS (m/z, %): 201 (MH⁺, 100).

### Synthesis of (9Z,12Z)-1-((R)-2-((1H-Indol-3-yl)methyl)pyrrolidin-1-yl)octadeca-9,12-dien-1-one (1-46)

A solution of linoleic acid (2.6 g, 9.28 mmol) in dry CH₂Cl₂ (20 mL) was treated with oxalyl chloride (1.04 mL, 12.38 mmol) followed by catalytic DMF (1 drop) at room temperature and stirred for additional 2 h. Solvent was evaporated to obtain crude acid chloride as a brown oil.

A solution of (R)-3-(pyrrolidin-2-ylmethyl)-1H-indole (0.62 g, 3.09 mmol) in dry DMF (15 mL) was treated with Et₃N (1.73 mL, 12.38 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound (R)—I-42 (1.1 g, 77%) as a light brown oil. ¹H NMR (CDCl₃): δ 8.15, 8.06 (2s, 1H), 7.88, 7.60 (2d, 1H, J=6.0 Hz), 7.40-7.28 (m, 1H), 7.27-7.14 (m, 2H), 7.03-7.01 (m, 1H), 5.43-5.34 (m, 4H), 4.52-4.45, 4.25-4.20 (2m, 1H), 3.60-3.30 (m, 3H), 3.05-2.20 (m, 3H), 2.30-2.15 (m, 2H), 2.10-2.00 (m, 4H), 1.95-1.73 (m, 5H), 1.42-1.27 (m, 15H), 0.93-0.89 (m, 3H); ESI-MS (m/z, %): 485 (M+Na), 463 (MH⁺, 100).

### Example 5: (9Z,12Z)—N-(2-(5-Methoxy-1H-indol-3-yl)ethyl)-N-methyloctadeca-9,12-dienamide (1-21)

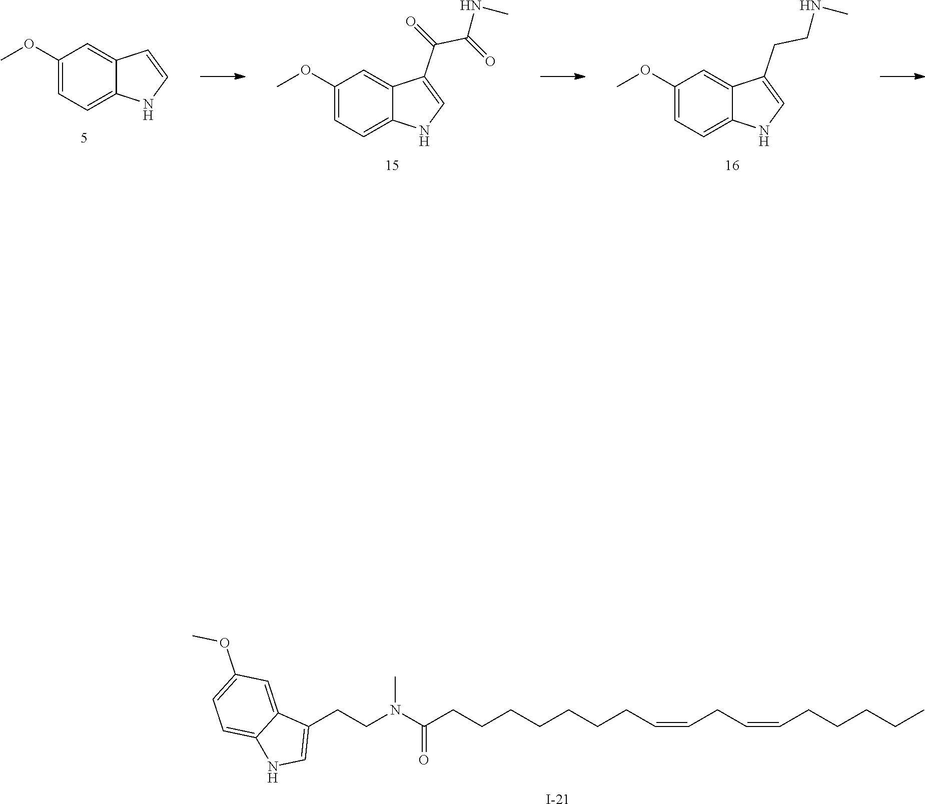

### Synthesis of 2-(5-methoxy-1H-indol-3-yl)-N-methyl-2-oxoacetamide (15)

A solution of 5-methoxy-1H-indole (3.0 g, 20.38 mmol) in dry THF (50 mL) was treated with oxalyl chloride (1.72 mL, 20.38 mmol) at 0° C. The reaction was brought to room temperature and stirred for additional 3 h. The reaction was cooled to 0° C., treated with methylamine solution (41 mL, 81.54 mmol, 2 M in THF) over a period of 5 min. The reaction was brought to room temperature and stirred for 24 h. The reaction was quenched with water (200 mL) and product was extracted into ethyl acetate (2×100 mL). Combined ethyl acetate layer was washed with brine (25 mL) and dried (Na₂SO₄). Solvent was evaporated and crude was treated with water (50 mL) and stirred for 15 min. Solid was filtered off, washed with water (2×15 mL) and dried on high vacuum to obtain the title compound 15 (4.2 g, 88.8%) as a yellow solid. ¹H NMR (DMSO-d₆): δ 12.10 (s, 1H), 8.70 (s, 1H), 8.64 (d, 1H, J=3.0 Hz), 7.74 (s, 1H), 7.42 (dd, 1H, J=3.0, 6.0 Hz), 6.89 (dd, 1H, J=1.5, 6.0 Hz), 3.80 (s, 1H), 2.74 (d, 3H, J=3.0 Hz), ESI-MS (m/z, %): 255 (M+Na, 100), 233 (MH⁺).

### Synthesis of 2-(5-methoxy-1H-indol-3-yl)-N-methylethan-1-amine (16)

A suspension of Lithium aluminum hydride (3.27 g, 86.12 mmol) in dry THF (50 mL) was treated with 2-(5-methoxy-1H-indol-3-yl)-N-methyl-2-oxoacetamide (2.5 g, 10.76 mmol) in dry THF (50 mL) at 0° C. over a period of 10 min. The reaction was brought to room temperature, then refluxed for additional 16 hours. The reaction was cooled to 0° C., then quenched with sequential addition of water (3.2 mL), 2 N NaOH solution (3.2 mL) and water (3.2 mL) over a period of 15 min. The reaction was brought to room temperature, stirred for additional 30 min. Solid was filtered off and washed with THF (2×50 mL). Combined THF layer was evaporated and crude was purified by column chromatography (2 M NH₃ in MeOH: CH₂Cl₂, 1:9) on silica gel to obtain the title compound 16 (0.81 g, 36.8%) as a yellow solid. ¹H NMR (DMSO-d₆): δ 10.62 (s, 1H), 7.22 (d, 1H, J=6.0 Hz), 7.09 (d, 1H, J=3.0 Hz), 6.99 (d, 1H, J=3.0 Hz), 6.71 (dd, 1H, J=1.5, 6.0 Hz), 3.76 (s, 3H), 2.81-2.72 (m, 4H), 2.33 (s, 3H); ESI-MS (m/z, %): 205 (MH⁺, 100).

### Synthesis of (9Z,12Z)—N-(2-(5-methoxy-1H-indol-3-yl)ethyl)-N-methyloctadeca-9,12-dienamide (1-21)

A solution of linoleic acid (3.29 g, 11.75 mmol) in dry CH₂Cl₂ (20 mL) was treated with oxalyl chloride (1.32 mL, 15.66 mmol) followed by catalytic DMF (1 drop) at room temperature and stirred for additional 2 h. Solvent was evaporated to obtain crude acid chloride as a brown oil.

A solution of 2-(5-methoxy-1H-indol-3-yl)-N-methylethan-1-amine (0.8 g, 3.92 mmol) in dry DMF (15 mL) was treated with Et₃N (2.18 mL, 15.66 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-21 (1.3 g, 71.2%) as a light-yellow glue. ¹H NMR (CDCl₃): δ 8.05, 8.00 (2s, 1H), 7.30-7.28 (m, 1H), 7.26-7.01 (m, 2H), 6.97-6.87 (m, 1H), 5.41-5.33 (m, 4H), 3.90 (s, 3H), 3.70 (t, 1H, J=6.0 Hz), 3.60 (t, 1H, J=6.0 Hz), 3.02-2.78 (m, 5H), 2.70-2.62 (m, 2H), 2.32 (t, 1H, J=6.0 Hz), 2.11-2.04 (m, 4H), 1.65-1.60 (m, 1H), 1.49-1.17 (m, 14H), 0.92 (t, 3H, J=6.0 Hz); ESI-MS (m/z, %): 489 (M+Na), 467 (MH⁺, 100).

### Example 6: (9Z,12Z)—N-(2-(1H-Indol-3-yl)ethyl)-N-methyloctadeca-9,12-dienamide (I-1)

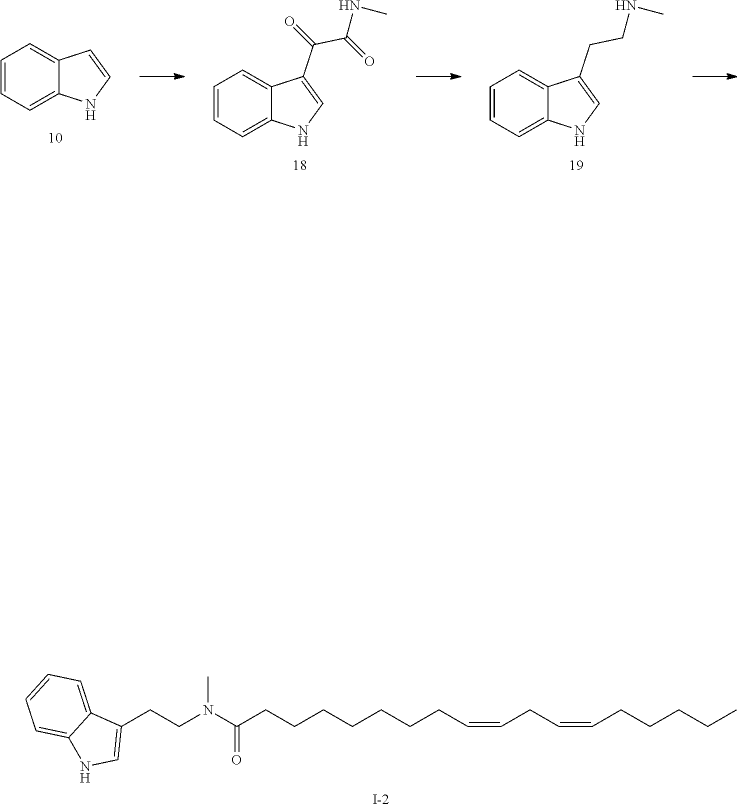

### Synthesis of 2-(1H-indol-3-yl)-N-methyl-2-oxoacetamide (18)

A solution of 1H-indole (3.0 g, 25.62 mmol) in dry THF (50 mL) was treated with oxalyl chloride (2.16 mL, 25.62 mmol) at 0° C. The reaction was brought to room temperature and stirred for additional 3 h. The reaction was cooled to 0° C., treated with methylamine solution (51.2 mL, 102.47 mmol, 2 M in THF) over a period of 5 min. The reaction was brought to room temperature and stirred for 24 h. The reaction was quenched with water (200 mL) and product was extracted into ethyl acetate (2×100 mL). Combined ethyl acetate layer was washed with brine (25 mL) and dried (Na₂SO₄). Solvent was evaporated and crude was treated with water (50 mL) and stirred for 15 min. Solid was filtered off, washed with water (2×15 mL) and dried on high vacuum to obtain the title compound 18 (4.4 g, 85%) as a yellow solid. ESI-MS (m/z, %): 203 (MH⁺, 100).

### Synthesis of 2-(1H-indol-3-yl)-N-methylethan-1-amine (19)

A suspension of Lithium aluminum hydride (3.75 g, 98.90 mmol) in dry THF (50 mL) was treated with 2-(1H-indol-3-yl)-N-methyl-2-oxoacetamide (2.5 g, 12.36 mmol) in dry THF (50 mL) at 0° C. over a period of 10 min. The reaction was brought to room temperature, then refluxed for additional 16 hours. The reaction was cooled to 0° C., then quenched with sequential addition of water (3.7 mL), 2 N NaOH solution (3.7 mL) and water (3.7 mL) over a period of 15 min. The reaction was brought to room temperature, stirred for additional 30 min. Solid was filtered off and washed with THF (2×50 mL). Combined THF layer was evaporated and crude was purified by column chromatography (2 M NH₃ in MeOH: CH₂Cl₂, 1:9) on silica gel to obtain the title compound 19 (0.64 g, 30%) as a yellow solid. ¹H NMR (CDCl₃): δ 8.26 (brs, 1H), 7.67 (dd, 1H, J=3.0, 6.0 Hz), 7.39 (dd, 1H, J=1.5, 6.0 Hz), 7.23 (t, 1H, J=6.0 Hz), 7.15 (t, 1H, J=6.0 Hz), 7.06 (s, 1H), 3.05-2.94 (m, 4H), 2.48 (s, 3H); ESI-MS (m/z, %): 175 (MH⁺, 100).

### Synthesis of (9Z,12Z)—N-(2-(1H-indol-3-yl)ethyl)-N-methyloctadeca-9,12-dienamide (I-1)

A solution of linoleic acid (3.08 g, 11.02 mmol) in dry CH₂Cl₂ (20 mL) was treated with oxalyl chloride (1.24 mL, 14.69 mmol) followed by catalytic DMF (1 drop) at room temperature and stirred for additional 2 h. Solvent was evaporated to obtain crude acid chloride as a brown oil.

A solution of 2-(1H-indol-3-yl)-N-methylethan-1-amine (0.64 g, 3.67 mmol) in dry DMF (15 mL) was treated with Et₃N (2.04 mL, 14.69 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-1 (1.26 g, 78.7%) as a brown glue. ¹H NMR (CDCl₃): δ 8.20, 8.15 (2s, 1H), 7.70, 7.60 (2d, 1H, J=6.0 Hz), 7.39 (t, 1H, J=6.0 Hz), 7.24-7.00 (m, 3H), 5.43-5.34 (m, 4H), 3.71 (t, 1H, J=6.0 Hz), 3.61 (t, 1H, J=6.0 Hz), 3.06-2.95 (m, 5H), 2.82-2.78 (m, 2H), 2.30 (t, 1H, J=6.0 Hz), 2.12-2.05 (m, 5H), 1.70-1-60 (m, 1H), 1.49-1.17 (m, 15H), 0.92 (t, 3H, J=6.0 Hz); ESI-MS (m/z, %): 459 (M+Na, 100), 437 (MH⁺).

### Example 7: (9Z,12Z)-1-(3-(5-Methoxy-1H-indol-3-yl)pyrrolidin-1-yl)octadeca-9,12-dien-1-one (1-81)

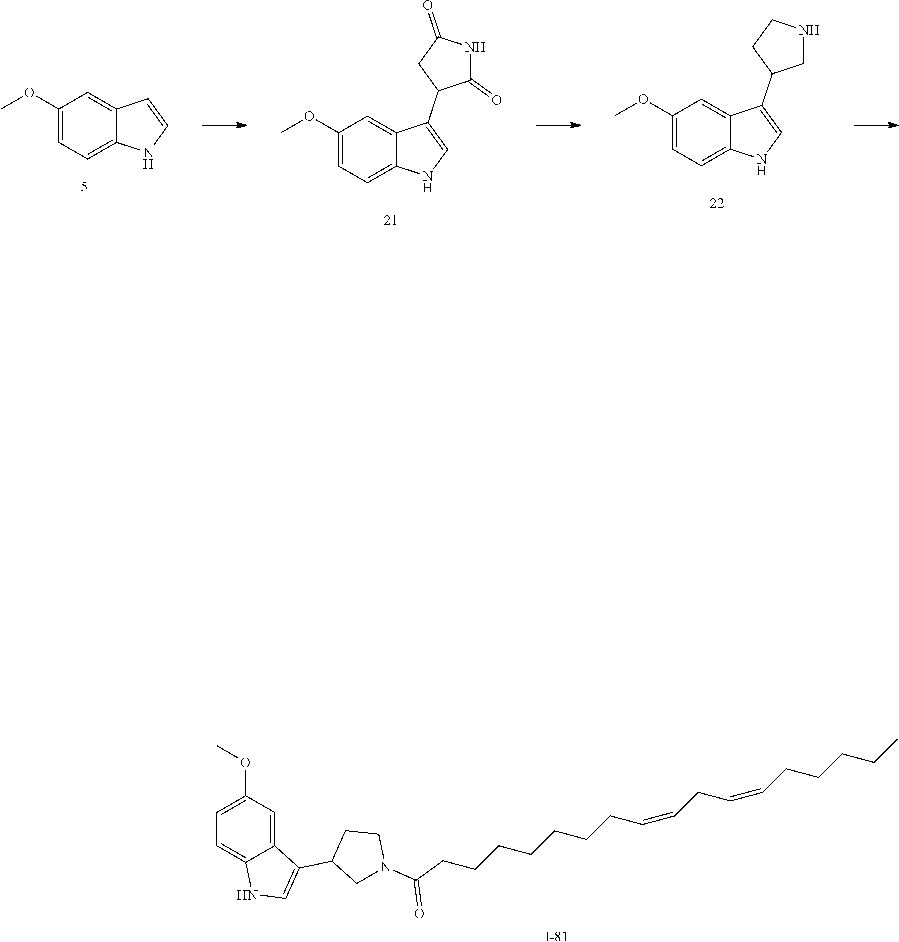

### Synthesis of 3-(5-methoxy-1H-indol-3-yl)pyrrolidine-2,5-dione (21)

A solution of 5-methoxy-1H-indole (7.73 g, 52.51 mmol), maleimide (5.6 g, 57.76 mmol) in acetic acid (50 mL) was refluxed for 3 days. The reaction was brought to room temperature, solvent was evaporated and crude was crystallized from a mixture of EtOAc:CH₂Cl₂, (1:9) to obtain the title compound 21 (12 g, 94%) as a yellow solid.

### Synthesis of 5-methoxy-3-(pyrrolidin-3-yl)-1H-indole (22)

A suspension of Lithium aluminum hydride (3.10 g, 81.88 mmol) in dry THF (50 mL) was treated with 3-(5-methoxy-1H-indol-3-yl)pyrrolidine-2,5-dione (2.5 g, 10.23 mmol) in dry THF (50 mL) at 0° C. over a period of 10 min. The reaction was brought to room temperature and then refluxed for additional 16 hours. The reaction was cooled to 0° C., then quenched with sequential addition of water (3.1 mL), 2 N NaOH solution (3.1 mL) and water (3.1 mL) over a period of 15 min. The reaction was brought to room temperature, stirred for additional 30 min. Solid was filtered off and washed with THF (2×50 mL). Combined THF layer was evaporated and crude was purified by column chromatography (2 M NH₃ in MeOH: CH₂Cl₂, 1:9) on silica gel to obtain the title compound 22 (1.0 g, 45.2%) as a yellow foam. ¹H NMR (DMSO-d₆): δ 10.63 (s, 1H), 7.22 (d, 1H, J=6.0 Hz), 7.11 (d, 1H, J=3.0 Hz), 7.02 (d, 1H, J=3.0 Hz), 6.72 (dd, 1H, J=3.0, 6.0 Hz), 3.76 (s, 3H), 3.36-3.24 (m, 2H), 3.03-2.91 (m, 2H), 2.76-2.71 (m, 1H), 2.20-2.12 (m, 1H), 1.84-1.75 (m, 1H).

### Synthesis of (9Z,12Z)-1-(3-(5-methoxy-1H-indol-3-yl)pyrrolidin-1-yl)octadeca-9,12-dien-1-one (1-81)

A solution of linoleic acid (1.94 g, 6.93 mmol) in dry CH₂Cl₂ (20 mL) was treated with oxalyl chloride (0.8 mL, 9.24 mmol) followed by catalytic DMF (1 drop) at room temperature and stirred for additional 2 h. Solvent was evaporated to obtain crude acid chloride as a brown oil.

A solution of 5-methoxy-3-(pyrrolidin-3-yl)-1H-indole (0.5 g, 2.31 mmol) in dry DMF (15 mL) was treated with Et₃N (1.3 mL, 9.24 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-81 (1.0 g, 91%) as a brown glue. ¹H NMR (CDCl₃): δ 8.20, 8.15 (2s, H), 7.32-7.28 (m, 1H), 7.06-7.00 (m, 2H), 6.93-6.88 (m, 1H), 5.42-5.34 (m, 4H), 4.20-3.52 (m, 9H), 2.82-2.77 (m, 2H), 2.52-2.31 (m, 3H), 2.10-2.04 (m, 5H), 1.71-1.67 (m, 2H), 1.42-1.29 (m, 13H), 0.93-0.89 (m, 3H); ESI-MS (m/z, %): 501 (M+Na, 100), 479 (MH⁺).

### Example 8: (9Z,12Z)-1-(3-(1H-Indol-3-yl)pyrrolidin-1-yl)octadeca-9,12-dien-1-one (I-84)

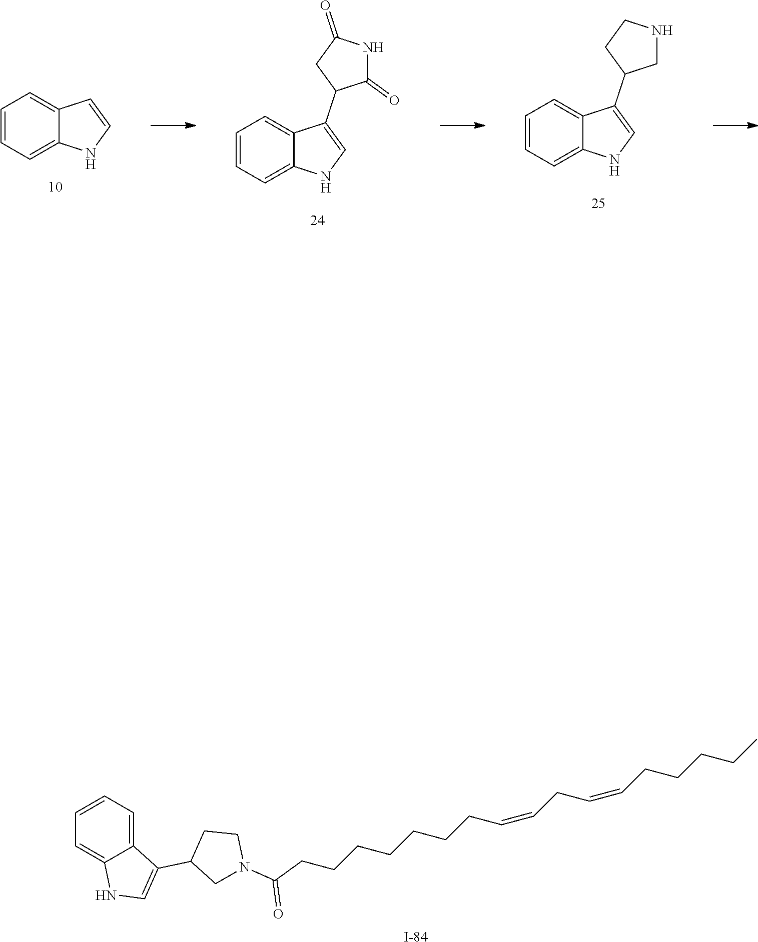

### Synthesis of 3-(1H-indol-3-yl)pyrrolidine-2,5-dione (24)

A solution of 1H-indole (12.5 g, 106.70 mmol), maleimide (11.4 g, 117.37 mmol) in acetic acid (75 mL) was refluxed for 3 days. The reaction was brought to room temperature, solvent was evaporated and crude was crystallized from a mixture of EtOAc: CH₂Cl₂, (1:9) to obtain the title compound 24 (14.8 g, 64.8%) as a yellow solid. ¹H NMR (DMSO-d₆): δ 11.31 (s, 1H), 11.04 (s, 1H), 7.44 (d, 1H, J=6.0 Hz), 7.40 (d, 1H, J=3.0 Hz), 7.34 (s, 1H), 7.14-7.10 (m, 1H), 7.03-6.99 (m, 1H), 4.37-4.33 (m, 1H), 3.24-3.16 (m, 1H), 2.79 (dd, 1H, J=3.0, 13.5 Hz).

### Synthesis of 3-(pyrrolidin-3-yl)-1H-indole (25)

A suspension of Lithium aluminum hydride (2.8 g, 74.68 mmol) in dry THF (50 mL) was treated with 3-(1H-indol-3-yl)pyrrolidine-2,5-dione (2.0 g, 9.33 mmol) in dry THF (50 mL) at 0° C. over a period of 10 min. The reaction was brought to room temperature and then refluxed for additional 16 hours. The reaction was cooled to 0° C., then quenched with sequential addition of water (2.8 mL), 2 N NaOH solution (2.8 mL) and water (2.8 mL) over a period of 15 min. The reaction was brought to room temperature, stirred for additional 30 min. Solid was filtered off and washed with THF (2×50 mL). Combined THF layer was evaporated and crude was purified by column chromatography (2 M NH₃ in MeOH:CH₂Cl₂, 1:9) on silica gel to obtain the title compound 25 (0.875 g, 50.3%) as a brown glue. ¹H NMR (DMSO-d₆): δ 10.78 (s, 1H), 7.55 (d, 1H, J=6.0 Hz), 7.33 (d, 1H, J=6.0 Hz), 7.14 (d, 1H, J=3.0 Hz), 7.08-7.04 (m, 1H), 6.98-6.94 (m, 1H), 3.38-3.23 (m, 3H), 3.02-2.90 (m, 2H), 2.76-2.72 (m, 1H), 2.19-2.11 (m, 1H), 1.85-1.76 (m, 1H).

### Synthesis of (9Z,12Z)-1-(3-(1H-indol-3-yl)pyrrolidin-1-yl)octadeca-9,12-dien-1-one (I-84)

A solution of linoleic acid (3.95 g, 14.09 mmol) in dry CH₂Cl₂ (20 mL) was treated with oxalyl chloride (1.59 mL, 18.79 mmol) followed by catalytic DMF (1 drop) at room temperature and stirred for additional 2 h. Solvent was evaporated to obtain crude acid chloride as a brown oil.

A solution of 3-(pyrrolidin-3-yl)-1H-indole (0.87 g, 4.69 mmol) in dry DMF (15 mL) was treated with Et₃N (2.6 mL, 18.79 mmol), followed by above crude acid chloride in dry DMF (15 mL) at 0° C. Over a period of 5 min. The reaction was brought to room temperature and stirred for additional 24 h. The reaction was quenched with water (200 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-84 (1.9 g, 91%) as a brown glue. ¹H NMR (CDCl₃): δ 8.25, 8.20 (2s, 1H), 7.65 (d, 1H, J=6.0 Hz), 7.41 (t, 1H, J=6.0 Hz), 7.26-7.23 (m, 1H), 7.19-7.15 (m, 1H), 7.04 (dd, 1H, J=3.0, 9.0 Hz), 5.42-5.33 (m, 4H), 4.10-3.52 (m, 5H), 2.82-2.78 (m, 2H), 2.50-2.04 (m, 9H), 1.72-1.66 (m, 2H), 1.41-1.28 (m, 13H), 0.93-0.89 (in, 3H); ESI-MS (m/z, %): 471 (M+Na), 449 (MH⁺).

### B. Biology

### Example 9: FLIPR Assay: Human 5-HT_{2A}

I. Assessment of the Activated Effect of Exemplary Compounds of Formula I Targeting on Human 5-HT_{2A} (h5-HT_{2A}) Receptor Under Agonist Mode:

### Compound Preparation and Assay Controls

I.a. Reagent and Materials:

| Regents | Vendor | Cat# |
|---|---|---|
| DMEM | Gibco | 10569010 |
| FBS | Hyclone | SH30406 |
| Penicillin-Streptomycin | Invitrogen | 15140 |
| Hygromycin B | Invivogen | Ant-hg-5 |
| G418 | Invitrogen | 11811031 |
| Tetracycline | Abcam | ab141223 |
| hydrochloride | | |
| DPBS | Gibco | 14190250 |
| DMSO | Millipore | 1029312500 |
| Probenecid | Sigma | P8761 |
| FLIPR Calcium 6 | Molecular | R8191 |
| Assay Kit | Device | |
| HEPES | Invitrogen | 15630 |
| Hank's Buffered | Invitrogen | 14025 |
| Saline Solution | | |
| Serotonin HCl | Selleck | S4244 |

I.b. Instrumentation and Consumables:

| Item | Supplier | Cat# |
|---|---|---|
| Fluorometric Imaging | Molecular | Tetra |
| Plate Reader (FLIPR) | Device | |
| Countess Automated | Invitrogen | Countess |
| Cell Counter | | |
| Cell Counting | Invitrogen | C10312 |
| Chamber Slides | | |
| STERI-CYCLE CO₂ | Thermo | 371 |
| Incubator | | |
| 1300 Series Class II | Thermo | 1389 |
| Biological Safety Cabinet | | |
| Table-type Large | Cence | L550 |
| Capacity Low Speed | | |
| Centrifuge | | |
| Centrifuge | Eppendorf | 5702 |
| Echo | Labcyte | 550 |
| Echo | Labcyte | 655 |
| Electro-thermal | Shanghai | DHP-9031 |
| incubator | Yiheng | |
| plate shaker | IKA | MS3 digital |
| Water Purification System | ULUPURE | UPH-III-20T |
| Versatile and Universal | Mettler | S220 |
| pH and Conductivity | Toledo | |
| Meters | | |
| 384-Well plate | Corning | 356663 |
| 384-Well LDV Clear | LABCYTE | LP-0200 |
| microplate | | |
| 384-Well Polypropylene | LABCYTE | PP-0200 |
| microplate | | |
| 384-well compound plate | Corning | 3657 |
| T25 cell culture flask | Corning | 430639 |
| 50 mL Polypropylene | JET | CFT011500 |
| Centrifuge Tube | | |
| 15 mL Polypropylene | JET | CFT011150 |
| Centrifuge Tube | | |

I.c. Experimental Methods and Procedures:
1. Cells are cultured in cell culture medium (DMEM containing 10% FBS, 1×penicillin-streptomycin 300 μg/ml G418 and 100 μg/ml hygromycin B) at 37° C., 5% (v/v) CO2.
2. One day before the assays, the cells are detached using TrypLE™ Express and cells are counted using cell counter. Only cells with >85% viability were used for the assay.
3. 20000 cells/well are seeded in 30 μl/well culture medium to a 384-well cell plate and cells are incubated overnight at 37° C., 5% (v/v) CO2.
4. On the assay day, 2×dye solution is prepared following the manual of the FLIPR® Calcium 6 Assay Kit: i. The dye is diluted with assay buffer (20 mM HEPES in 1×HBSS, PH7.4); ii. Probenecid was added to the final concentration of 5 mM; iii. Vortexed vigorously for 1-2 minutes.
5. Medium is removed from cell plate by flicking the cell plate on towel papers.
6. 10 μl of assay buffer and 10 μl of 2×dye solution is added to each well of the cell plate.
7. The cell plate is placed on plate shaker, the plate was agitated at 600 rpm for 2 minutes. The plate is incubated at 37° C. for 2 hours followed by additional 15-minute incubation at 25° C.
8. 3×compound in assay buffer is prepared: a. Reference compounds are diluted to required concentration with DMSO. The compounds were added to a 384-well compound plate; b. Serial dilutions are performed; c. 10 mM test compounds are added to the compound plate, and 3-fold serial dilutions were performed. d. Transferred 60 nl/well of compounds from source plate to a 384-well compound plate (Corning, 3657) by using an Echo; e. Add 20 μl/well assay buffer to the compound plate; f. Mix the plate on plate shaker for 2 mins;
9. The cell plate, compound plate and tips are put into FLIPR, 10 μl of 3×compound is transferred to the cell plate per well with FLIPR.

### Data Analysis

i. The normalized fluorescence reading (RFU) is calculated as shown follow, while Fmax and Fmin stand for maximum and minimum of calcium signal during defined time window: RFU=Fmax−Fmin
ii. Calculate the percentage activation by using following equation:

$$\% Activation = \frac{( RFUcompound - RFU low control )}{( \begin{matrix}RFU top concentration of reference agonist - \\ RFU low con trol\end{matrix} )} \times 100 \%$$

iii. Calculate EC₅₀ by fitting % activation against log of compound concentrations with Hill equation using XLfit.

The compounds of the application will be found to be 5-HT_{2A} agonists. The results of representative compounds will be presented as EC50 provided in Table 1. The letter “A” indicates an EC50<10,000 nM; “B” indicates an EC50>10,000 nM but <30,000 nM; and “C” indicates an EC50>30,000 nM.

TABLE 1
Effect of exemplary compounds of Formula I targeting
on human 5-HT_{2A} (h5-HT_{2A}) receptor under agonist mode

| | h5-HT_{2A} |
|---|---|
| Compound ID# | EC₅₀ [nM] |
| 5-MeO-DMT | A |
| DMT | A |
| I-1 | C |
| I-21 | C |
| I-46 | C |
| I-55 | C |
| I-81 | C |
| I-84 | C |
| I-87 | C |
| I-88 | C |

### Results & Discussion

Exemplary compounds of Formula I are evaluated functionally using FLIPR assay for their effect on h5-HT_{2A} receptor under agonist mode. EC₅₀ (nM) concentrations are illustrated in Table 1. This assay will confirm that compounds of the application are not effective direct inhibitors of the target human 5-HT_{2A} receptors.

### II. Human 5-HT_{2A}: Radioligand Binding Assay:

### II.1. Materials and Instruments:

| Materials | Vendor | Cat# |
|---|---|---|
| Ketanserin Hydrochloride, | PerkinElmer | NET791250UC |
| [Ethylene-3H]- | | |
| Ketanserin | MedChemExpress | HY-10562 |
| Bovine Serum Albumin (BSA) | Sigma | A1933 |
| Calcium chloride | Sigma | C5670 |
| (CaCl₂) | | |
| Tris(hydroxymethyl)amino- | Alfa Aesar | A18494 |
| methane (Tris) | | |
| Polyethylenimine, | Sigma | 408727 |
| branched (PEI) | | |

### II.2. Instrumentation and Consumables:

| Item | Supplier | Cat# |
|---|---|---|
| Microbeta² Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene | Labcyte | PP-0200 |
| Microplate | | |
| 96 Round Well Plate | Corning | 3799 |
| 96 Round Deep | Axygen | P-DW-11-C |
| Well Plate | | |
| Echo | LABCYTE | 550 |

### II.3 Experiment Procedure:

i. Prepare the assay buffer following the table below;

| Reagent | Concentration |
|---|---|
| Tris | 50 mM |
| CaCl₂ | 4 mM |
| BSA | 0.1% (w/v) |

Adjust pH to 7.4 followed by 0.2 μM sterile filtration

ii. Preparation of 8 doses of reference and test compounds starting from 10 mM stock solution as requested by 5-fold serial dilutions with 100%;
iii. Prepare (v/v) DMSO: a. 50 μl/well of 0.5% (v/v) PEI is added to UniFilter-96 GF/B plates. The plates are sealed and incubated at 4° C. for 3 hrs; b. After incubation, the plates are washed 3 times with ice-cold wash buffer (50 mM Tris, pH7.4);
iv. Preparation of assay plates: a. Cell membrane are diluted with assay buffer and 330 μl/well is added to 96 round deep well plates to reach a concentration of 20 μg/well; b. 8 concentrations of reference or test compounds are prepared and 110 μl/well is added to 96 round deep well plates; c. [3H]-ketanserin is diluted with assay buffer to 5 nM (5× final concentration) and 110 μl/well is added to 96 round deep well plates.
v. The plate is centrifuged at 1000 rpm for 30 secs and then agitated at 600 rpm, R.T. for 5 min.
vi. The plates are sealed and incubates at 27° C. for 90 min.
vii. The incubation is stopped by vacuum filtration onto GF/B filter plates followed by 4 times washing with ice-cold wash buffer (50 mM Tris, pH7.4).
viii. The plates are dried at 37° C. for 45 min.
ix. The filter plates are sealed and 40 μl/well of scintillation cocktail is added.
x. The plate is read by using a Microbeta2 microplate counter.

### Data Analysis:

For reference and test compounds, the results are expressed as % Inhibition, using the normalization equation: N=100−100×(U−C2)/(C1−C2), where U is the unknown value, C1 is the average of high controls, and C2 is the average of low controls. The IC50 is determined by fitting percentage of inhibition as a function of compound concentrations with Hill equation using XLfit.

### Results and Discussion:

The results of potential competition binding properties of the representative compounds targeting on human 5-hydroxytryptamine receptors 2A (5-HT_{2A}) will be summarized in Table 2. The results of representative compounds will be presented as IC₅₀ provided in Table 2. The letter “A” indicates an EC50<10,000 nM; “B” indicates an IC50>10,000 nM but <30,000 nM; and “C” indicates an EC50>30,000 nM.

TABLE 2
Effect of exemplary compounds of Formula I using Radioligand
binding assay on human 5-HT_{2A} receptor

| Compound ID# | h5-HT_{2A} IC₅₀ [nM] |
|---|---|
| 5-MeO-DMT | A |
| DMT | A |
| I-1 | B |
| I-21 | B |
| I-46 | C |
| I-55 | C |
| I-81 | C |
| I-84 | B |
| I-87 | B |
| I-88 | B |

### Example 10: Psychedelic-Like Effect of Compounds of Formula I

The effect of different doses of representative compounds of Formula I was evaluated on head-twitch response (HTR) and other behavioural responses indicative of serotonin syndrome as behavior-based models of psychedelic activity.

### Protocols

### Mouse Head Twitch

Male, C57BL/6J mice (body weight range 20-30 g) were dosed with the appropriate dose of test article, and following a 1-minute pre-treatment time, placed in individual observation chambers. Animals were visually assessed for the incidence head twitches continuously over a 1 hr period. Head twitches were defined as a rapid jerk of the head which was not elicited by an external tactile stimulus (Come and Pickering, Psychopharmacologia, 1967, 11(1): 65-78). Each head twitch was individually counted by a trained observer, and the data expressed as the mean±SEM of 6-10 mice per group. Mice were used in a single experiment only.

### Results and Discussion

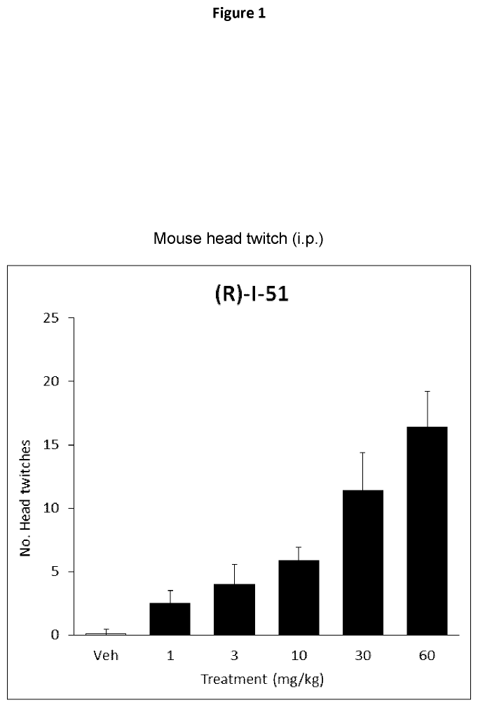

Exemplary compound (R)—I-51 (1-60 mg/kg SC) elicited a robust head twitch response expression that was clearer compared to 5-MeO DMT (Cpd. 32) (FIG. 1). The ED₅₀ for compound (R)—I-51 was 2.8 mg/kg compared to an ED₅₀ of 8 mg/kg SC for 5-MeO-DMT (Cpd. 32). The duration of HT expression at 10-30 mg/kg was slightly longer for compound (R)—I-51 compared to 5-MeO DMT (0-30 min vs. 0-20 min). In marked contrast to 5-MeO-DMT (Cpd. 32), 1-51 did not induce signs of 5-HT syndrome—i.e. whole body twitches, FPT, Straub tail—up to 60 mg/kg SC (FIG. 1). Although not formally measured, compound (R)—I-51 (1-30 mg/kg) treatment did not reduce motor activity (FIG. 1). If any thing, test subjects seemed more active.

While the present application has been described with reference to examples, it is to be understood that the scope of the claims should not be limited by the embodiments set forth in the examples, but should be given the broadest interpretation consistent with the description as a whole.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the application described and claimed herein.

1. A compound of Formula I: 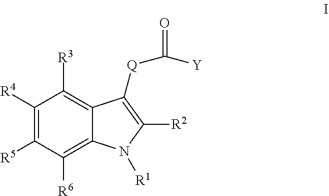
or a pharmaceutically acceptable salt, solvate and/or prodrug thereof,
wherein:
R¹ is selected from H and C₁₋₆alkyl;
Q is selected from Q1, Q2, Q3, Q4 and Q5: 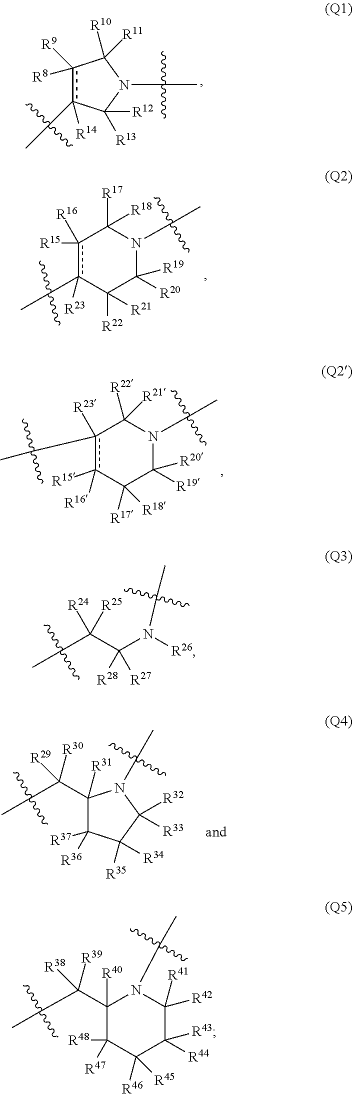 [symbol]
is a single bond or a double bond provided that when [symbol] in Q1 is a double bond then R⁸ and R¹⁴ are not present, when [symbol] in Q2 is a double bond then R¹⁶ and R²³ are not present and when [symbol] in Q2′ is a double bond R^{16′} and R^{23′} are not present; [symbol]
over a bond means that the bond is attached to a remaining portion of the compound, wherein the [symbol] over the bond attached to the N in Q1, Q2, Q3, Q4 and Q5 indicates the bond that is attached to C(O)—Y and the [symbol] over the bond attached to the C in Q1, Q2, Q3, Q4 and Q5 indicates the bond that is attached to the indole;
Y is selected from R⁴⁹, O—R⁴⁹ and O—C₁₋₄alkylene-O—C(O)—R⁴⁹;
R⁴⁹ is selected from C₇₋₃₀alkyl and C₇₋₃₀alkenyl;
R² is selected from H, halo, C₁₋₆alkyl and C₁₋₆alkoxy;
one of R³ and R⁴ is selected from H, OH, halo, C₁₋₆alkyl and C₁₋₆alkoxy and the other of R³ and R⁴ is selected from H, OH, halo, C₁₋₆alkyl, C₁₋₆alkoxy and OP(O)(OR⁵⁰)₂;
R⁵ and R⁶ are independently selected from H, OH, halo, C₁₋₆alkyl and C₁₋₆alkoxy;
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R¹⁵, R^{16′}, R^{17′}, R^{18′}, R^{19′}, R^{20′}, R^{21′}, R^{22′}, R^{23′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, halo and C₁₋₆alkyl;
R²⁶ is selected from H and C₁₋₆alkyl;
R⁵⁰ is selected from H and C₁₋₆alkyl; and
all available hydrogen atoms are optionally and independently replaced with a fluorine atom and/or all available atoms are optionally replaced with alternate isotope thereof,
provided that
(1) when Q is Q3, either (i) R²⁶ is C₁₋₆alkyl or (ii) Y is selected from O—R⁴⁹ and O—C₁₋₄alkylene-O—C(O)—R⁴⁹, or (iii) the compound of Formula I comprises at least one deuterium, and
(2) when Q is Q2, [symbol] is a single bond and R¹⁵, R¹⁶, R¹⁷, R¹⁸, R¹⁹, R²⁰, R²¹, R²² and R²³ are all H, then either (i) Y is selected from O—R⁴⁹ and O—C₁₋₄alkylene-O—C(O)—R⁴⁹, or (ii) the compound of Formula I comprises at least one deuterium.

2. The compound of claim 1, wherein Y is selected from R⁴⁹, O—R⁴⁹ and O—C₁alkylene-O—C(O)—R⁴⁹.

3. The compound of claim 1 or 2, wherein the alkyl or alkene group of R⁴⁹ is an alkyl or alkene group present in a fatty acid selected from:

| Common Name | Lipid Number | Chemical Name |
|---|---|---|
| linoleic acid (LA) | 18:2 (n-6) | all-cis-9,12- |
| | | octadecadienoic acid, |
| rumenic acid | 18:2 (n-6) | 9Z,11E-octadecadienoic |
| (conjugated linoleic acid) | | acid, |
| conjugated | 18:2 (n-6) | 10E,12Z-octadecadienoic |
| linoleic acid | | acid, |
| conjugated | 18:2 (n-6) | 9Z,12E-octadecadienoic |
| linoleic acid | | acid, |
| gamma-linolenic | 18:3 (n-6) | all-cis-6,9,12- |
| acid (GLA) | | octadecatrienoic acid, |
| calendic acid | 18:3 (n-6) | 8E,10E,12Z- |
| | | octadecatrienoic acid, |
| eicosadienoic acid | 20:2 (n-6) | all-cis-11,14- |
| | | eicosadienoic acid, |
| dihomo-gamma-linolenic | 20:3 (n-6) | all-cis-8,11,14- |
| acid (DGLA) | | eicosatrienoic acid, |
| arachidonic | 20:4 (n-6) | all-cis-5,8,11,14- |
| acid (AA, ARA) | | eicosatetraenoic acid |
| docosadienoic acid | 22:2 (n-6) | all-cis-13,16- |
| | | docosadienoic acid, |
| adrenic acid | 22:4 (n-6) | all-cis-7,10,13,16- |
| | | docosatetraenoic acid, |
| osbond acid | 22:5 (n-6) | all-cis-4,7,10,13,16- |
| | | docosapentaenoic acid, |
| tetracosatetraenoic | 24:4 (n-6) | all-cis-9,12,15,18- |
| acid | | tetracosatetraenoic acid, |
| tetracosapentaenoic | 24:5 (n-6) | all-cis-6,9,12,15,18- |
| acid | | tetracosapentaenoic acid, |
| α-linolenic | 18:3 (n-3) | all-cis-9,12,15- |
| acid (ALA) | | octadecatrienoic acid, |
| stearidonic | 18:4 (n-3) | all-cis-6,9,12,15- |
| acid (SDA) | | octadecatetraenoic acid, |
| hexadecatrienoic | 16:3 (n-3) | all-cis-7,10,13- |
| acid (HTA) | | hexadecatrienoic acid, |
| eicosatrienoic | 20:3 (n-3) | all-cis-11,14,17- |
| acid (ETE) | | eicosatrienoic acid, |
| eicosatetraenoic | 20:4 (n-3) | all-cis-8,11,14,17- |
| acid (ETA) | | eicosatetraenoic acid, |
| eicosapentaenoic | 20:5 (n-3) | all-cis-5,8,11,14,17- |
| acid (EPA) | | eicosapentaenoic acid, |
| heneicosapentaenoic | 21:5 (n-3) | all-cis-6,9,12,15,18- |
| acid (HPA) | | heneicosapentaenoic |
| | | acid, |
| docosapentaenoic | 22:5 (n-3) | all-cis-7,10,13,16,19- |
| acid (DPA) | | docosapentaenoic acid, |
| docosahexaenoic | 22:6 (n-3) | all-cis-4,7,10,13,16,19- |
| acid (DHA) | | docosahexaenoic acid, |
| tetracosapentaenoic | 24:5 (n-3) | all-cis-9,12,15,18,21- |
| acid | | tetracosapentaenoic acid, |
| tetracosahexaenoic | 24:6 (n-3) | all-cis-6,9,12,15,18,21- |
| acid (Nisinic acid) | | tetracosahexaenoic acid, |
| myristoleic acid | 14:1 (n-5) | 9Z-tetradecenoic acid, |
| palmitoleic acid | 16:1 (n-7) | (9Z)-hexadecenoic acid, |
| sapienic acid | 16:1 (n-10) | (6Z)-hexadecenoic acid, |
| oleic acid | 18-1 (n-9) | (9Z)-octadecenoic acid, |
| elaidic acid | 18:1 (n-9) | (E)-octadecenoic acid, |
| vaccenic acid | 18:1 (n-7) | (11E)-octadecenoic acid, |
| eruric acid | 22-1 (n-9) | (13Z)-Docosenoic acid, |
| caprylic acid | 8:0 | octanoic acid, |
| capric acid | 10:0 | decanoic acid, |
| lauric acid | 12:0 | dodecanoic acid, |
| myristic acid | 14:0 | tetradecanoic acid, |
| palmitic acid | 16:0 | hexadecenoic acid, |
| stearic acid | 18:0 | octadecanoic acid, |
| arachidic acid | 20:0 | Icosanoic acid, |
| behenic acid | 22:0 | docosanoic acid, |
| lignoceric acid | 24:0 | tetracosanoic acid, and |
| cerotic acid | 26:0 | hexacosanoic acid. |

4. The compound of claim 3, wherein R⁴⁹ is C₁₃₋₂₁alkyl, wherein all available hydrogen atoms are optionally and independently replaced with a fluorine atom or deuterium atom.

5. The compound of claim 1 or 2, wherein R⁴⁹ C₁₀₋₂₅alkenyl, wherein all available hydrogen atoms are optionally and independently replaced with a fluorine atom or deuterium atom.

6. The compound of claim 5, wherein R⁴⁹ is selected from (CH₂)₇CH═CH(CH₂)₇CH₃, (CH₂)₇CH═CHCH₂CH═CH(CH₂)₄CH₃, (CH₂)₈CH═CHCH₂CH═CH(CH₂)₄CH₃, (CH₂)₇CH═CHCH₂CH═CHCH₂CH═CHCH₂CH₃, (CH₂)₃CH═CHCH₂CH═CHCH₂CH═CHCH₂CH═CH(CH₂)₃CH₃, (CH₂)₂CH═CHCH₂CH═CHCH₂CH═CHCH₂CH═CHCH₂CH═CHCH₂CH═CH(CH₂)₁CH₃.

7. The compound of any one of claims 1 to 6, wherein Y is R⁴⁹.

8. The compound of any one of claims 1 to 6, wherein Y is OR⁴⁹ or O—C₁₋₄alkylene-O—C(O)—R⁴⁹.

9. The compound of claim 1, wherein the compound of Formula I has the following structure: 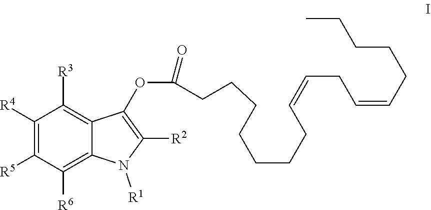
or a pharmaceutically acceptable salt and/or solvate thereof.

10. The compound of any one of claims 1 to 9, wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R^{15′}, R^{16′}, R^{17′}, R^{18′}, R^{19′}, R^{20′}, R^{21′}, R^{22′}, R^{23′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H, D, F, Cl, C₁₋₆alkyl, C₁₋₆fluoroalkyl and C₁₋₆deuteroalkyl.

11. The compound of claim 10, wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R^{15′}, R^{16′}, R^{17′}, R^{18′}, R^{19′}, R^{20′}, R^{21′}, R^{22′}, R^{23′}, R²⁴, R²⁵, R²⁷, R²⁸, R²⁹, R³⁰, R³¹, R³², R³³, R³⁴, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ are independently selected from H and D.

12. The compound of any one of claims 1 to 11, wherein Q is selected from 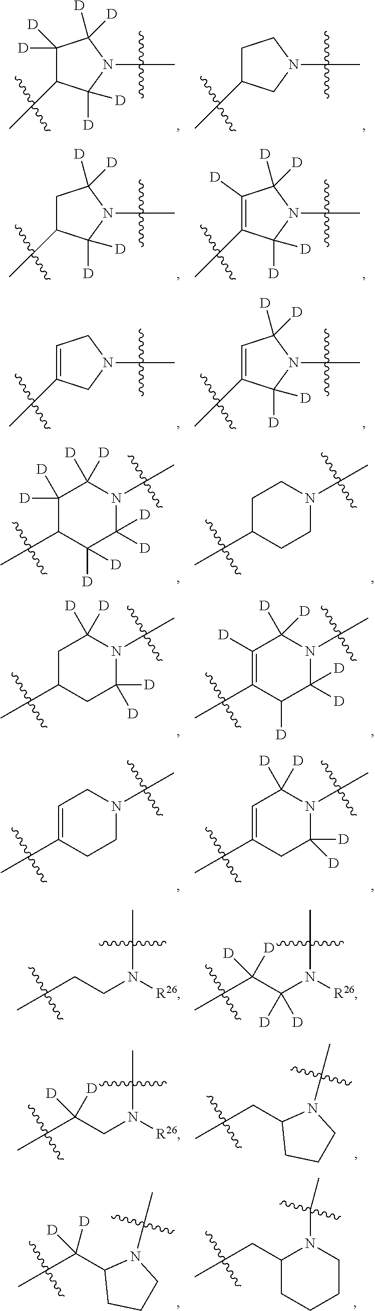 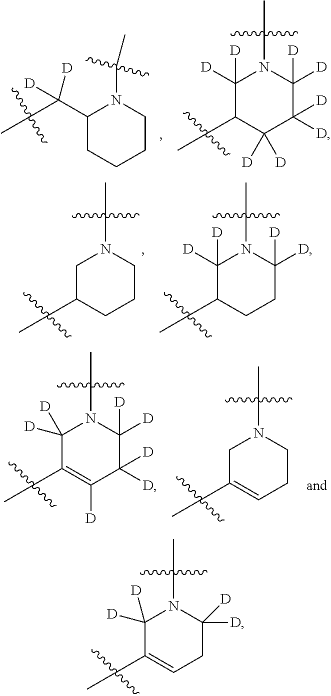
wherein R²⁶ is selected from H, D, C₁₋₆alkyl, C₁₋₆fluoroalkyl and C₁₋₆deuteroalkyl.

13. The compound of claim 12, wherein R²⁶ is selected from H and CH₃.

14. The compound of any one of claims 1 to 13, wherein R¹ is H.

15. The compound of any one of claims 1 to 14, wherein R², R⁵ and R⁶ are independently H, D or F.

16. The compound of claim 15, wherein R², R⁵ and R⁶ are H or D.

17. The compound of any one of claims 1 to 16, wherein R³ is selected from H, D, F, OH, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy.

18. The compound of claim 17, wherein R³ is H.

19. The compound of any one of claims 1 to 18, wherein R⁴ is selected from H, D, F, OH, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy.

20. The compound of claim 19, wherein R⁴ is selected from H, D, F, Cl, CH₃, CH₃CH₂, CH₃CH₂O, (CH₃)₂CH, (CH₃)₂CHO, CF₃, CF₂H, CD₃, CH₃O, CF₃O, CHF₂O, and CD₃O.

21. The compound of claim 20, wherein R⁴ is CH₃O.

22. The compound of claim 20, wherein R⁴ is H.

23. The compound of claim 1 selected from:

| Compound I.D | Chemical Structure |
|---|---|
| I-1 | 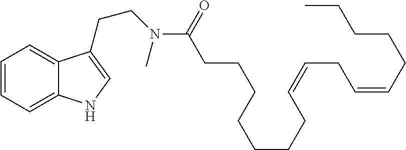 [SMILES: CCCCC/C=C\C/C=C\CCCCCCCC(=O)N(C)CCc1c[nH]c2ccccc12] |
| I-2 | 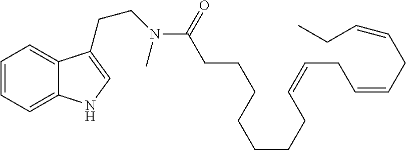 [SMILES: CC/C=C\C/C=C\C/C=C\CCCCCCCC(=O)N(C)CCc1c[nH]c2ccccc12] |
| I-3 | 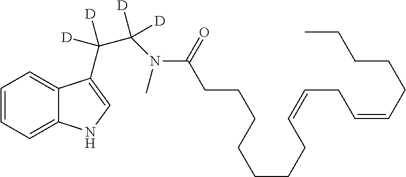 [SMILES: [2H]C([2H])(c1c[nH]c2ccccc12)C([2H])([2H])N(C)C(=O)CCCCCCC/C=C\C/C=C\CCCCC] |
| I-4 | 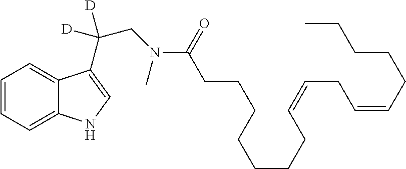 [SMILES: [2H]C([2H])(CN(C)C(=O)CCCCCCC/C=C\C/C=C\CCCCC)c1c[nH]c2ccccc12] |
| I-5 | 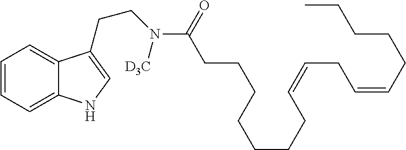 [SMILES: [2H]C([2H])([2H])N(CCc1c[nH]c2ccccc12)C(=O)CCCCCCC/C=C\C/C=C\CCCCC] |
| I-6 | 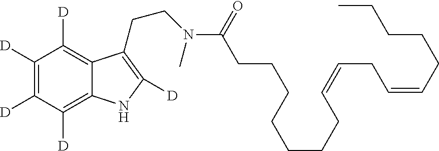 [SMILES: [2H]c1[nH]c2c([2H])c([2H])c([2H])c([2H])c2c1CCN(C)C(=O)CCCCCCC/C=C\C/C=C\CCCCC] |
| I-7 | 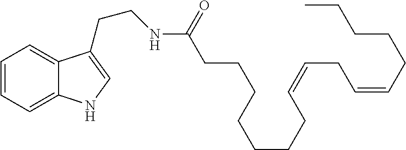 [SMILES: CCCCC/C=C\C/C=C\CCCCCCCC(=O)NCCc1c[nH]c2ccccc12] |
| I-8 | 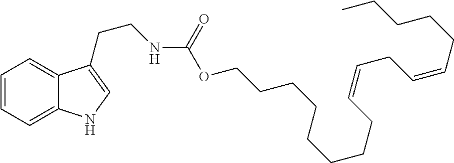 [SMILES: CCCCC/C=C\C/C=C\CCCCCCCCOC(=O)NCCc1c[nH]c2ccccc12] |
| I-9 | 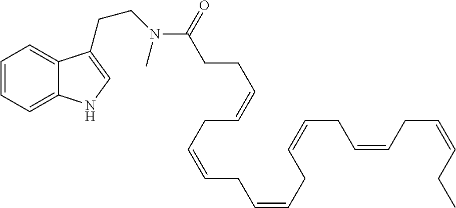 [SMILES: CC/C=C\C/C=C\C/C=C\C/C=C\C/C=C\C/C=C\CCC(=O)N(C)CCc1c[nH]c2ccccc12] |
| I-10 | 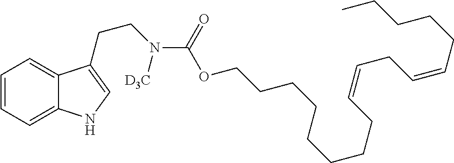 [SMILES: [2H]C([2H])([2H])N(CCc1c[nH]c2ccccc12)C(=O)OCCCCCCCC/C=C\C/C=C\CCCCC] |
| I-11 | 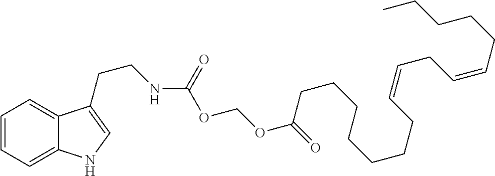 [SMILES: CCCCC/C=C\C/C=C\CCCCCCCC(=O)OCOC(=O)NCCc1c[nH]c2ccccc12] |
| I-12 | 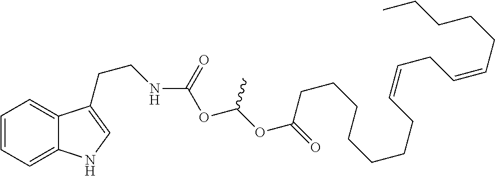 [SMILES: CCCCC/C=C\C/C=C\CCCCCCCC(=O)OC(C)OC(=O)NCCc1c[nH]c2ccccc12] |
| I-13 | 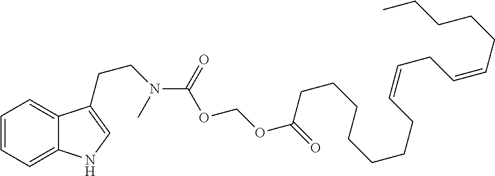 [SMILES: CCCCC/C=C\C/C=C\CCCCCCCC(=O)OCOC(=O)N(C)CCc1c[nH]c2ccccc12] |
| I-14 | 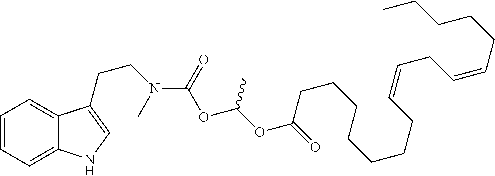 [SMILES: CCCCC/C=C\C/C=C\CCCCCCCC(=O)OC(C)OC(=O)N(C)CCc1c[nH]c2ccccc12] |
| I-15 | 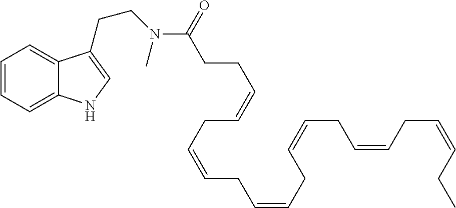 [SMILES: CC/C=C\C/C=C\C/C=C\C/C=C\C/C=C\C/C=C\CCC(=O)N(C)CCc1c[nH]c2ccccc12] |
| I-16 | 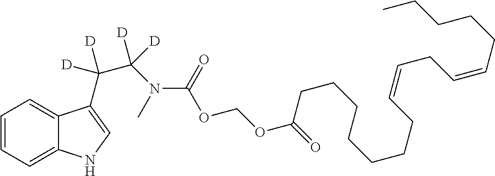 [SMILES: [2H]C([2H])(c1c[nH]c2ccccc12)C([2H])([2H])N(C)C(=O)OCOC(=O)CCCCCCC/C=C\C/C=C\CCCCC] |
| I-17 | 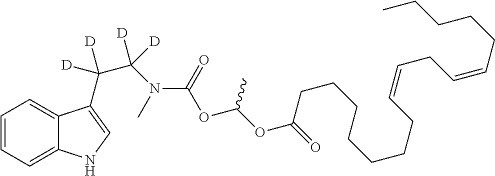 [SMILES: [2H]C([2H])(c1c[nH]c2ccccc12)C([2H])([2H])N(C)C(=O)OC(C)OC(=O)CCCCCCC/C=C\C/C=C\CCCCC] |
| I-18 | 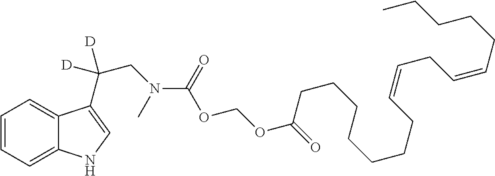 [SMILES: [2H]C([2H])(CN(C)C(=O)OCOC(=O)CCCCCCC/C=C\C/C=C\CCCCC)c1c[nH]c2ccccc12] |
| I-19 | 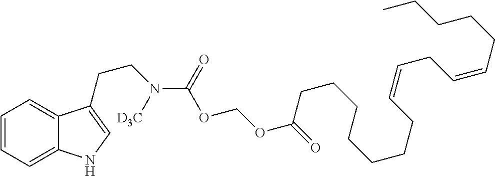 [SMILES: [2H]C([2H])([2H])N(CCc1c[nH]c2ccccc12)C(=O)OCOC(=O)CCCCCCC/C=C\C/C=C\CCCCC] |
| I-20 | 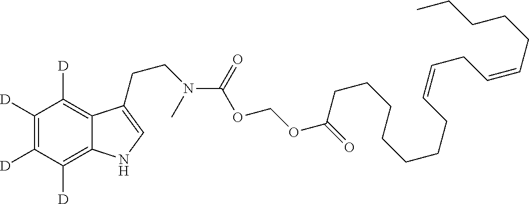 [SMILES: [2H]c1c([2H])c([2H])c2c(CCN(C)C(=O)OCOC(=O)CCCCCCC/C=C\C/C=C\CCCCC)c[nH]c2c1[2H]] |
| I-21 | 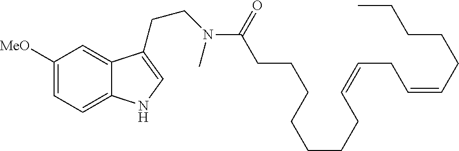 [SMILES: CCCCC/C=C\C/C=C\CCCCCCCC(=O)N(C)CCc1c[nH]c2ccc(OC)cc12] |
| I-22 | 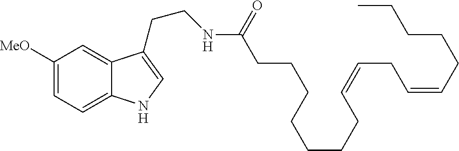 [SMILES: CCCCC/C=C\C/C=C\CCCCCCCC(=O)NCCc1c[nH]c2ccc(OC)cc12] |
| I-23 | 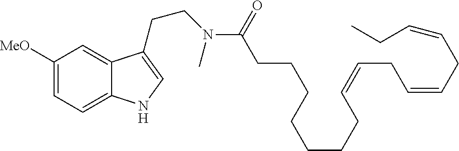 [SMILES: CC/C=C\C/C=C\C/C=C\CCCCCCCC(=O)N(C)CCc1c[nH]c2ccc(OC)cc12] |
| I-24 | 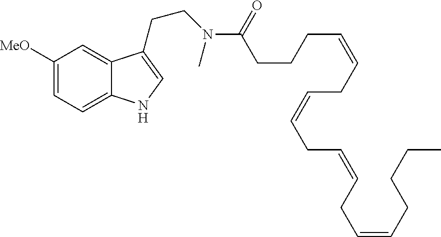 [SMILES: CCCC/C=C\C/C=C\C/C=C\C/C=C\CCCC(=O)N(C)CCc1c[nH]c2ccc(OC)cc12] |
| I-25 | 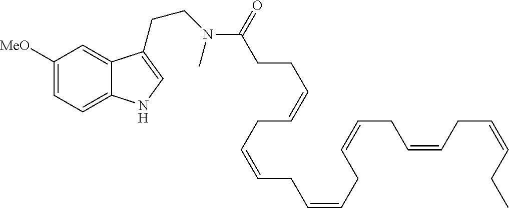 [SMILES: CC/C=C\C/C=C\C/C=C\C/C=C\C/C=C\C/C=C\CCC(=O)N(C)CCc1c[nH]c2ccc(OC)cc12] |
| I-26 | 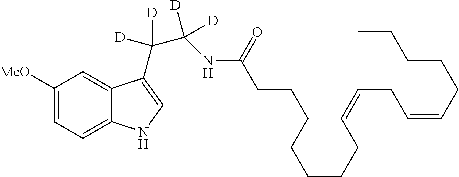 [SMILES: [2H]C([2H])(NC(=O)CCCCCCC/C=C\C/C=C\CCCCC)C([2H])([2H])c1c[nH]c2ccc(OC)cc12] |
| I-27 | 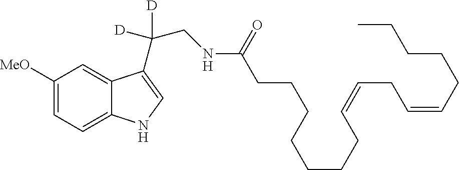 [SMILES: [2H]C([2H])(CNC(=O)CCCCCCC/C=C\C/C=C\CCCCC)c1c[nH]c2ccc(OC)cc12] |
| I-28 | 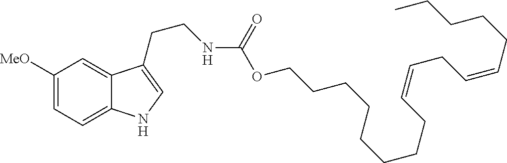 [SMILES: CCCCC/C=C\C/C=C\CCCCCCCCOC(=O)NCCc1c[nH]c2ccc(OC)cc12] |
| I-29 | 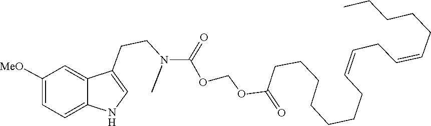 [SMILES: CCCCC/C=C\C/C=C\CCCCCCCC(=O)OCOC(=O)N(C)CCc1c[nH]c2ccc(OC)cc12] |
| I-30 | 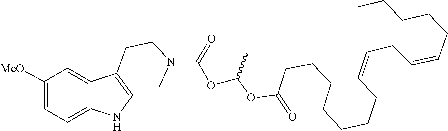 [SMILES: CCCCC/C=C\C/C=C\CCCCCCCC(=O)OC(C)OC(=O)N(C)CCc1c[nH]c2ccc(OC)cc12] |
| I-31 | 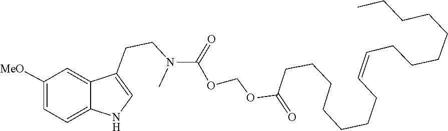 [SMILES: CCCCCCCC/C=C\CCCCCCCC(=O)OCOC(=O)N(C)CCc1c[nH]c2ccc(OC)cc12] |
| I-32 | 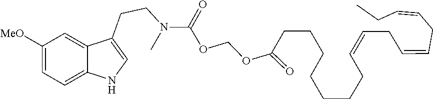 [SMILES: CC/C=C\C/C=C\C/C=C\CCCCCCCC(=O)OCOC(=O)N(C)CCc1c[nH]c2ccc(OC)cc12] |
| I-33 | 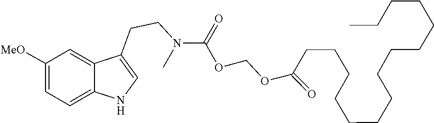 [SMILES: CCCCCCCCCCCCCCCC(=O)OCOC(=O)N(C)CCc1c[nH]c2ccc(OC)cc12] |
| I-34 | 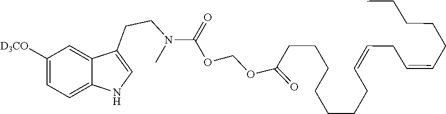 |
| I-35 | 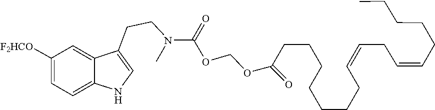 [SMILES: CCCCC/C=C\C/C=C\CCCCCCCC(=O)OCOC(=O)N(C)CCc1c[nH]c2ccc(OC(F)F)cc12] |
| I-36 | 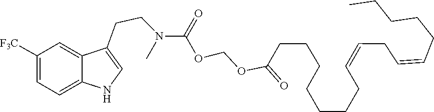 [SMILES: CCCCC/C=C\C/C=C\CCCCCCCC(=O)OCOC(=O)N(C)CCc1c[nH]c2ccc(C(F)(F)F)cc12] |
| I-37 | 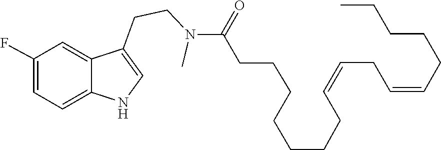 [SMILES: CCCCC/C=C\C/C=C\CCCCCCCC(=O)N(C)CCc1c[nH]c2ccc(F)cc12] |
| I-38 | 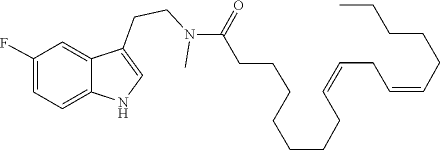 [SMILES: CCCCC/C=C\C/C=C\CCCCCCCC(=O)N(C)CCc1c[nH]c2ccc(F)cc12] |
| I-39 | 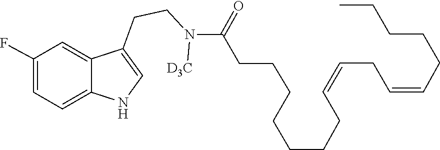 [SMILES: [2H]C([2H])([2H])N(CCc1c[nH]c2ccc(F)cc12)C(=O)CCCCCCC/C=C\C/C=C\CCCCC] |
| I-40 | 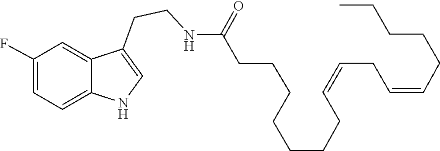 [SMILES: CCCCC/C=C\C/C=C\CCCCCCCC(=O)NCCc1c[nH]c2ccc(F)cc12] |
| I-41 | 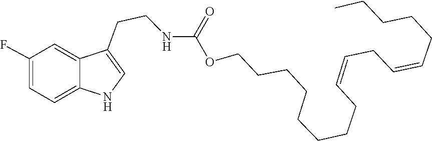 [SMILES: CCCCC/C=C\C/C=C\CCCCCCCCOC(=O)NCCc1c[nH]c2ccc(F)cc12] |
| I-42 | 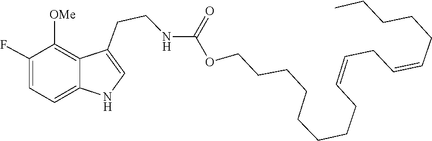 [SMILES: CCCCC/C=C\C/C=C\CCCCCCCCOC(=O)NCCc1c[nH]c2ccc(F)c(OC)c12] |
| I-43 | 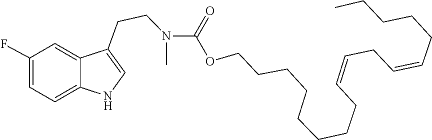 [SMILES: CCCCC/C=C\C/C=C\CCCCCCCCOC(=O)N(C)CCc1c[nH]c2ccc(F)cc12] |
| I-44 | 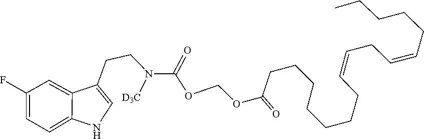 [SMILES: [2H]C([2H])([2H])N(CCc1c[nH]c2ccc(F)cc12)C(=O)OCOC(=O)CCCCCCC/C=C\C/C=C\CCCCC] |
| (R)-I-45 | 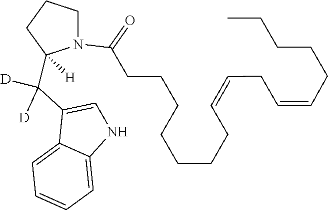 [SMILES: [2H]C([2H])(c1c[nH]c2ccccc12)[C@H]1CCCN1C(=O)CCCCCCC/C=C\C/C=C\CCCCC] |
| (R)-I-46 | 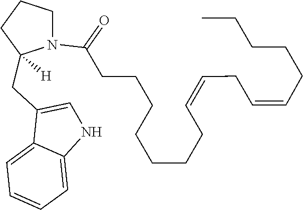 [SMILES: CCCCC/C=C\C/C=C\CCCCCCCC(=O)N1CCC[C@@H]1Cc1c[nH]c2ccccc12] |
| (R)-I-47 | 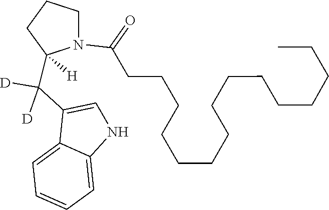 [SMILES: [2H]C([2H])(c1c[nH]c2ccccc12)[C@H]1CCCN1C(=O)CCCCCCCCCCCCCCC] |
| (R)-I-48 | 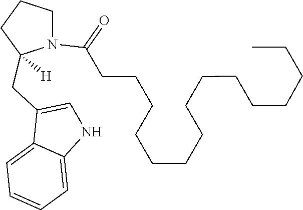 [SMILES: CCCCCCCCCCCCCCCC(=O)N1CCC[C@@H]1Cc1c[nH]c2ccccc12] |
| (R)-I-49 | 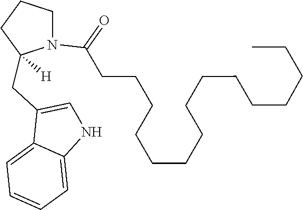 [SMILES: CCCCCCCCCCCCCCCC(=O)N1CCC[C@@H]1Cc1c[nH]c2ccccc12] |
| (R)-I-50 | 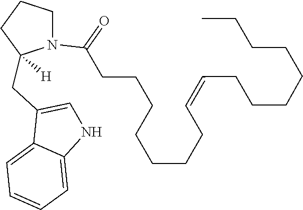 [SMILES: CCCCCCCC/C=C\CCCCCCCC(=O)N1CCC[C@@H]1Cc1c[nH]c2ccccc12] |
| (R)-I-51 | 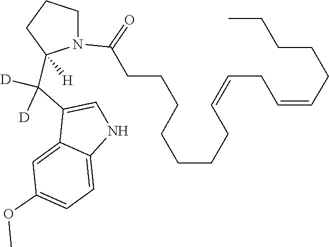 [SMILES: [2H]C([2H])(c1c[nH]c2ccc(OC)cc12)[C@H]1CCCN1C(=O)CCCCCCC/C=C\C/C=C\CCCCC] |
| (R)-I-52 | 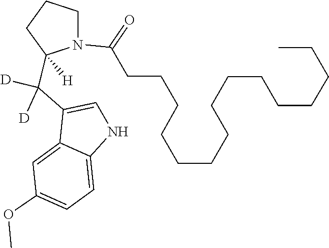 [SMILES: [2H]C([2H])(c1c[nH]c2ccc(OC)cc12)[C@H]1CCCN1C(=O)CCCCCCCCCCCCCCCC] |
| (R)-I-53 | 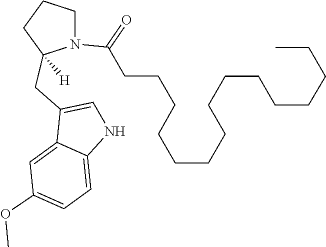 [SMILES: CCCCCCCCCCCCCCCC(=O)N1CCC[C@@H]1Cc1c[nH]c2ccc(OC)cc12] |
| (R)-I-54 | 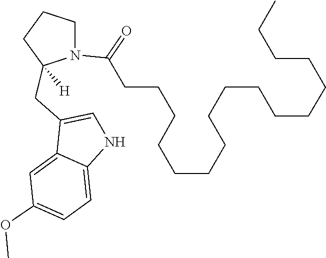 [SMILES: CCCCCCCCCCCCCCCCCCC(=O)N1CCC[C@@H]1Cc1c[nH]c2ccc(OC)cc12] |
| (R)-I-55 | 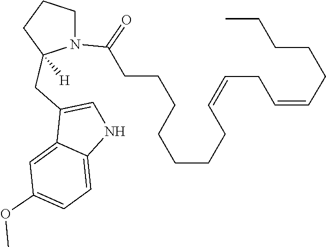 [SMILES: CCCCC/C=C\C/C=C\CCCCCCCC(=O)N1CCC[C@@H]1Cc1c[nH]c2ccc(OC)cc12] |
| (R)-I-56 | 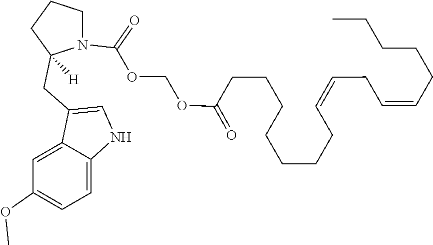 [SMILES: CCCCC/C=C\C/C=C\CCCCCCCC(=O)OCOC(=O)N1CCC[C@@H]1Cc1c[nH]c2ccc(OC)cc12] |
| (R)-I-57 | 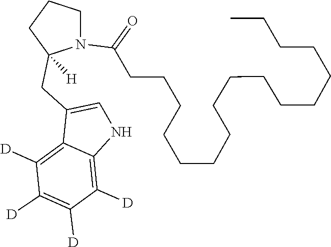 [SMILES: [2H]c1c([2H])c([2H])c2c(C[C@H]3CCCN3C(=O)CCCCCCCCCCCCCCCCC)c[nH]c2c1[2H]] |
| (R)-I-58 | 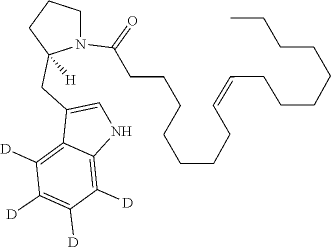 [SMILES: [2H]c1c([2H])c([2H])c2c(C[C@H]3CCCN3C(=O)CCCCCCC/C=C\CCCCCCCC)c[nH]c2c1[2H]] |
| (R)-I-59 | 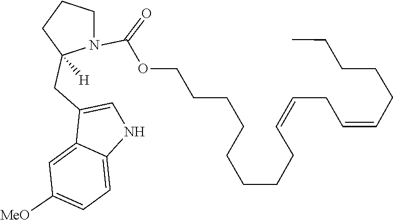 [SMILES: CCCCC/C=C\C/C=C\CCCCCCCCOC(=O)N1CCC[C@@H]1Cc1c[nH]c2ccc(OC)cc12] |
| (R)-I-60 | 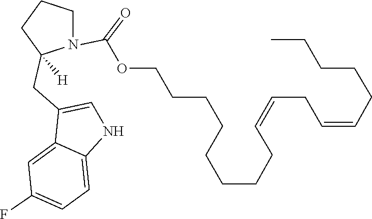 [SMILES: CCCCC/C=C\C/C=C\CCCCCCCCOC(=O)N1CCC[C@@H]1Cc1c[nH]c2ccc(F)cc12] |
| (R)-I-61 | 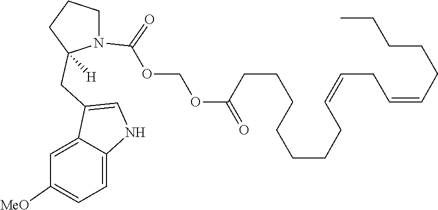 [SMILES: CCCCC/C=C\C/C=C\CCCCCCCC(=O)OCOC(=O)N1CCC[C@@H]1Cc1c[nH]c2ccc(OC)cc12] |
| (R)-I-62 | 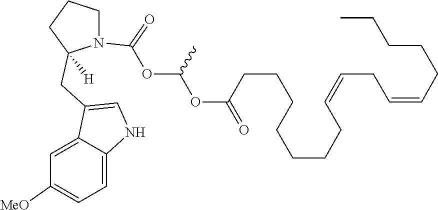 [SMILES: CCCCC/C=C\C/C=C\CCCCCCCC(=O)OC(C)OC(=O)N1CCC[C@@H]1Cc1c[nH]c2ccc(OC)cc12] |
| (R)-I-63 | 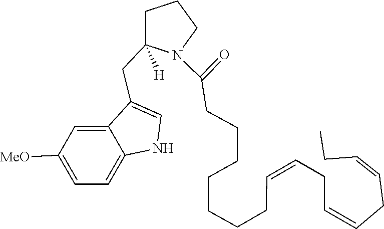 |
| (S)-I-64 | 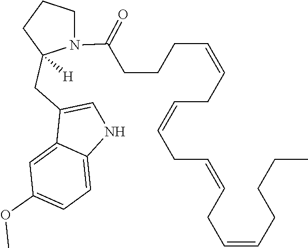 [SMILES: CCCC/C=C\C/C=C\C/C=C\C/C=C\CCCC(=O)N1CCC[C@@H]1Cc1c[nH]c2ccc(OC)cc12] |
| (R)-I-65 | 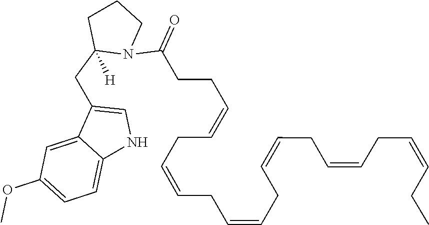 [SMILES: CC/C=C\C/C=C\C/C=C\C/C=C\C/C=C\C/C=C\CCC(=O)N1CCC[C@@H]1Cc1c[nH]c2ccc(OC)cc12] |
| (R)-I-66 | 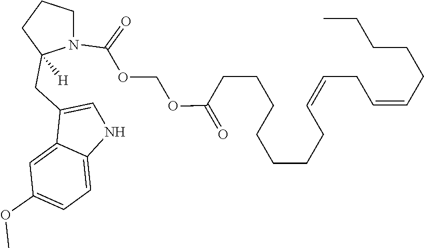 [SMILES: CCCCC/C=C\C/C=C\CCCCCCCC(=O)OCOC(=O)N1CCC[C@@H]1Cc1c[nH]c2ccc(OC)cc12] |
| (R)-I-67 | 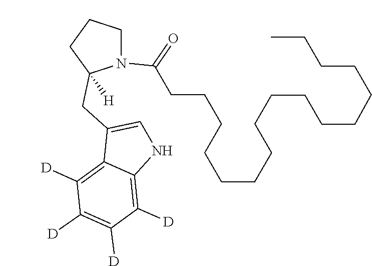 |
| (R)-I-68 | 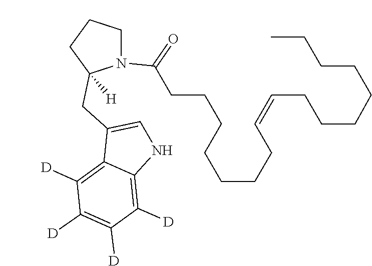 |
| (R)-I-69 | 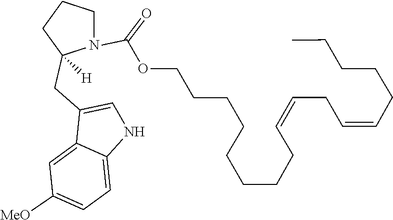 [SMILES: CCCCC/C=C\C/C=C\CCCCCCCCOC(=O)N1CCC[C@@H]1Cc1c[nH]c2ccc(OC)cc12] |
| (R)-I-70 | 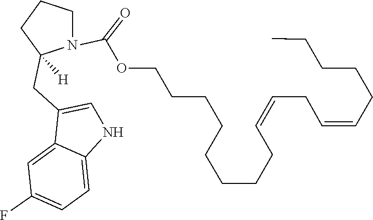 [SMILES: CCCCC/C=C\C/C=C\CCCCCCCCOC(=O)N1CCC[C@@H]1Cc1c[nH]c2ccc(F)cc12] |
| (R)-I-71 | 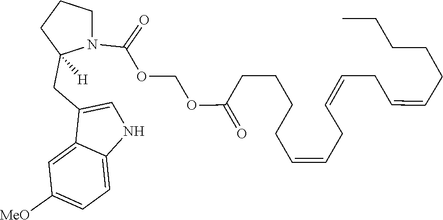 [SMILES: CCCCC/C=C\C/C=C\C/C=C\CCCCC(=O)OCOC(=O)N1CCC[C@@H]1Cc1c[nH]c2ccc(OC)cc12] |
| (R)-I-72 | 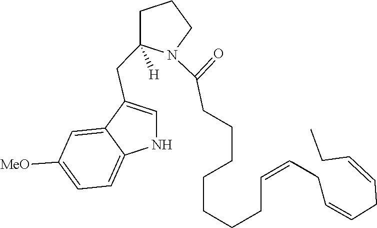 |
| (R)-I-73 | 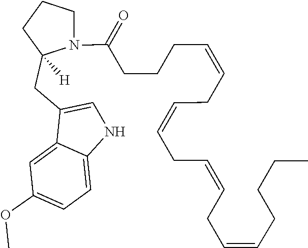 [SMILES: CCCC/C=C\C/C=C\C/C=C\C/C=C\CCCC(=O)N1CCC[C@@H]1Cc1c[nH]c2ccc(OC)cc12] |
| (R)-I-74 | 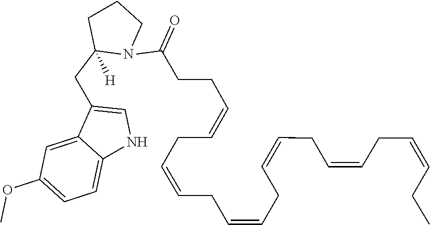 [SMILES: CC/C=C\C/C=C\C/C=C\C/C=C\C/C=C\C/C=C\CCC(=O)N1CCC[C@@H]1Cc1c[nH]c2ccc(OC)cc12] |
| I-75 | 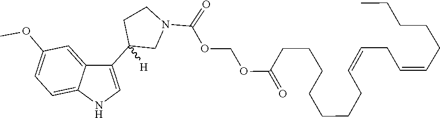 [SMILES: CCCCC/C=C\C/C=C\CCCCCCCC(=O)OCOC(=O)N1CCC(c2c[nH]c3ccc(OC)cc23)C1] |
| (R)-I-76 | 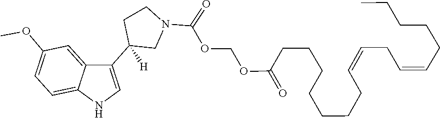 [SMILES: CCCCC/C=C\C/C=C\CCCCCCCC(=O)OCOC(=O)N1CC[C@H](c2c[nH]c3ccc(OC)cc23)C1] |
| (R)-I-77 | 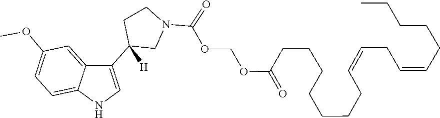 [SMILES: CCCCC/C=C\C/C=C\CCCCCCCC(=O)OCOC(=O)N1CC[C@@H](c2c[nH]c3ccc(OC)cc23)C1] |
| (R)-I-78 | 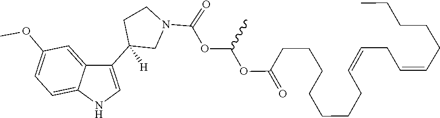 [SMILES: CCCCC/C=C\C/C=C\CCCCCCCC(=O)OC(C)OC(=O)N1CC[C@H](c2c[nH]c3ccc(OC)cc23)C1] |
| (R)-I-79 | 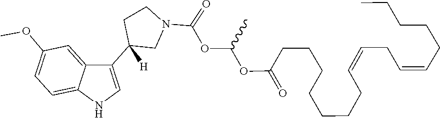 [SMILES: CCCCC/C=C\C/C=C\CCCCCCCC(=O)OC(C)OC(=O)N1CC[C@@H](c2c[nH]c3ccc(OC)cc23)C1] |
| I-80 | 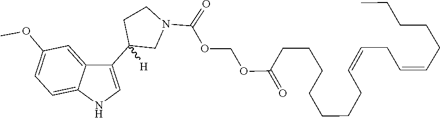 [SMILES: CCCCC/C=C\C/C=C\CCCCCCCC(=O)OCOC(=O)N1CCC(c2c[nH]c3ccc(OC)cc23)C1] |
| I-81 | 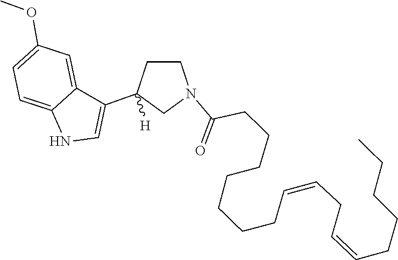 [SMILES: CCCCC/C=C\C/C=C\CCCCCCCC(=O)N1CCC(c2c[nH]c3ccc(OC)cc23)C1] |
| (R)-I-82 | 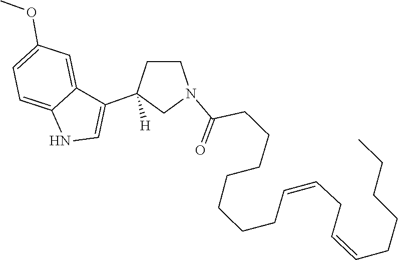 [SMILES: CCCCC/C=C\C/C=C\CCCCCCCC(=O)N1CC[C@H](c2c[nH]c3ccc(OC)cc23)C1] |
| (R)-I-83 | 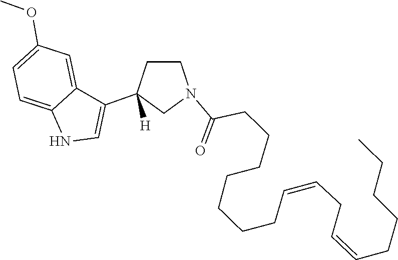 [SMILES: CCCCC/C=C\C/C=C\CCCCCCCC(=O)N1CC[C@@H](c2c[nH]c3ccc(OC)cc23)C1] |
| I-84 | 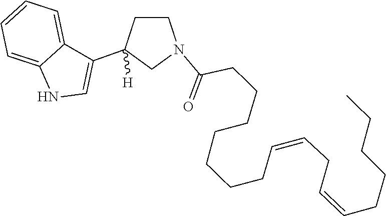 [SMILES: CCCCC/C=C\C/C=C\CCCCCCCC(=O)N1CCC(c2c[nH]c3ccccc23)C1] |
| (R)-I-85 | 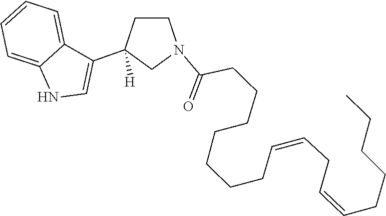 [SMILES: CCCCC/C=C\C/C=C\CCCCCCCC(=O)N1CC[C@H](c2c[nH]c3ccccc23)C1] |
| (R)-I-86 | 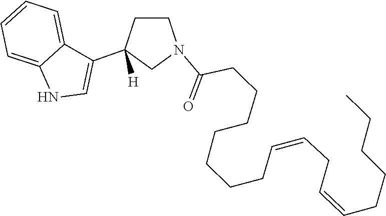 [SMILES: CCCCC/C=C\C/C=C\CCCCCCCC(=O)N1CC[C@@H](c2c[nH]c3ccccc23)C1] |
| I-87 | 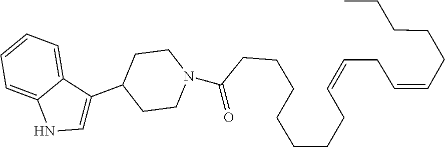 [SMILES: CCCCC/C=C\C/C=C\CCCCCCCC(=O)N1CCC(c2c[nH]c3ccccc23)CC1] |
| I-88 | 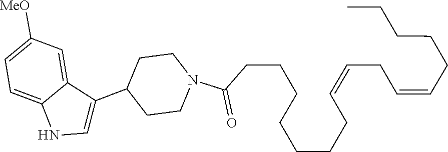 [SMILES: CCCCC/C=C\C/C=C\CCCCCCCC(=O)N1CCC(c2c[nH]c3ccc(OC)cc23)CC1] |
| I-89 | 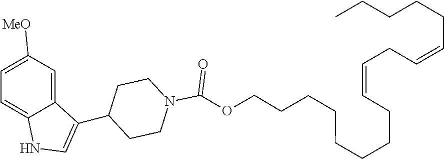 [SMILES: CCCCC/C=C\C/C=C\CCCCCCCCOC(=O)N1CCC(c2c[nH]c3ccc(OC)cc23)CC1] |
| I-90 | 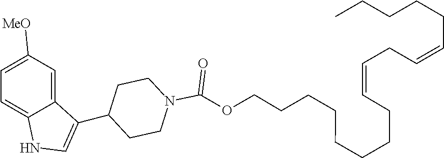 [SMILES: CCCCC/C=C\C/C=C\CCCCCCCCOC(=O)N1CCC(c2c[nH]c3ccc(OC)cc23)CC1] |
| I-91 | 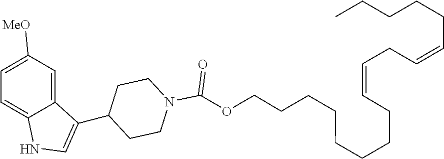 [SMILES: CCCCC/C=C\C/C=C\CCCCCCCCOC(=O)N1CCC(c2c[nH]c3ccc(OC)cc23)CC1] |
| I-92 | 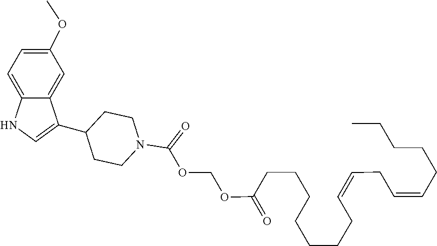 [SMILES: CCCCC/C=C\C/C=C\CCCCCCCC(=O)OCOC(=O)N1CCC(c2c[nH]c3ccc(OC)cc23)CC1] |
| I-93 | 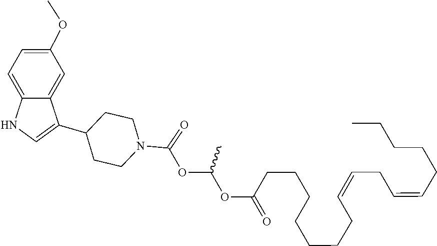 [SMILES: CCCCC/C=C\C/C=C\CCCCCCCC(=O)OC(C)OC(=O)N1CCC(c2c[nH]c3ccc(OC)cc23)CC1] |
| I-94 | 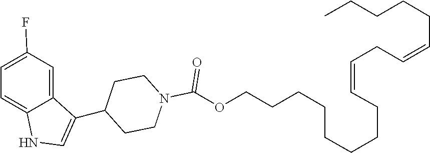 [SMILES: CCCCC/C=C\C/C=C\CCCCCCCCOC(=O)N1CCC(c2c[nH]c3ccc(F)cc23)CC1] |
| I-95 | 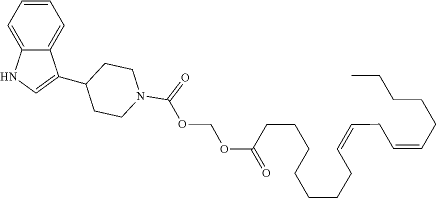 [SMILES: CCCCC/C=C\C/C=C\CCCCCCCC(=O)OCOC(=O)N1CCC(c2c[nH]c3ccccc23)CC1] |
| I-96 | 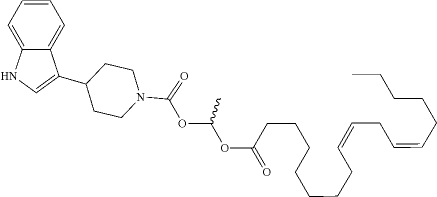 [SMILES: CCCCC/C=C\C/C=C\CCCCCCCC(=O)OC(C)OC(=O)N1CCC(c2c[nH]c3ccccc23)CC1] |
| I-97 | 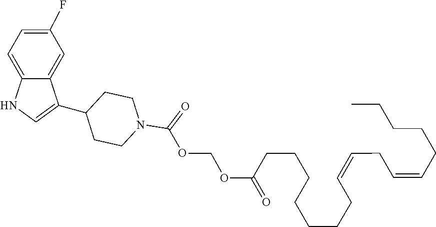 [SMILES: CCCCC/C=C\C/C=C\CCCCCCCC(=O)OCOC(=O)N1CCC(c2c[nH]c3ccc(F)cc23)CC1] |
| I-98 | 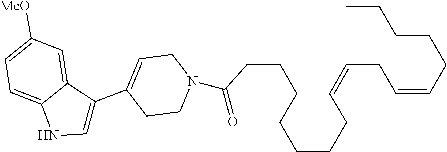 [SMILES: CCCCC/C=C\C/C=C\CCCCCCCC(=O)N1CC=C(c2c[nH]c3ccc(OC)cc23)CC1] |
| (S)-I-99 | 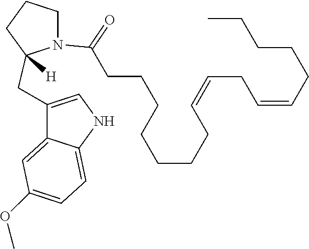 [SMILES: CCCCC/C=C\C/C=C\CCCCCCCC(=O)N1CCC[C@H]1Cc1c[nH]c2ccc(OC)cc12] |
| (S)-I-100 | 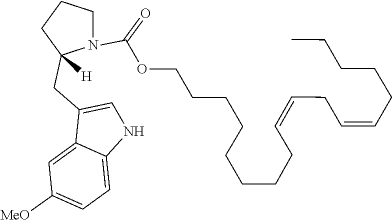 [SMILES: CCCCC/C=C\C/C=C\CCCCCCCCOC(=O)N1CCC[C@H]1Cc1c[nH]c2ccc(OC)cc12] |
| (S)-I-101 | 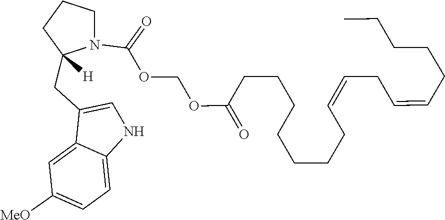 [SMILES: CCCCC/C=C\C/C=C\CCCCCCCC(=O)OCOC(=O)N1CCC[C@H]1Cc1c[nH]c2ccc(OC)cc12] |
| (R)-I-102 | 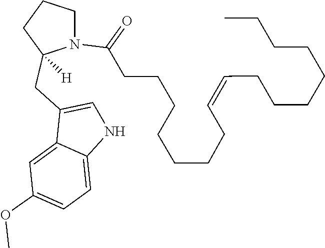 [SMILES: CCCCCCCC/C=C\CCCCCCCC(=O)N1CCC[C@@H]1Cc1c[nH]c2ccc(OC)cc12] |
| | and |
| (S)-I-103 | 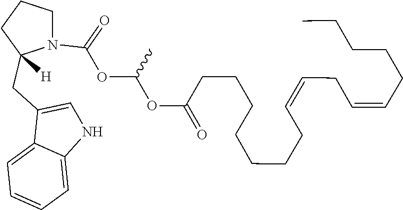 [SMILES: CCCCC/C=C\C/C=C\CCCCCCCC(=O)OC(C)OC(=O)N1CCC[C@H]1Cc1c[nH]c2ccccc12] |

or a pharmaceutically acceptable salt, solvate and/or prodrug thereof.

24. A composition comprising one or more compounds of any one of claims 1 to 23 and a carrier.

25. A pharmaceutical composition comprising one or more compounds of any one of claims 1 to 23 and pharmaceutically acceptable carrier.

26. A composition comprising one or more compounds of any one of claims 1 to 23 and one or more components of a nano-carrier system.

27. The composition of claim 26, wherein the nano-carrier system is selected from liposomes, micelles, nanoparticles, nano-emulsions and lipidic nano-systems.

28. A method of treating a disease, disorder or condition by activation of a serotonin receptor comprising administering a therapeutically effective amount of one or more compounds of any one of claims 1 to 23 to a subject in need thereof.

29. A method of treating a mental illness comprising administering a therapeutically effective amount of any one of claims 1 to 23 to a subject in need thereof.

30. The method of claim 29, wherein the mental illness is selected from hallucinations and delusions and a combination thereof.

31. The method of claim 29, wherein the mental illness is selected anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; and somatic symptom disorders and combinations thereof.

32. A method of treating psychosis or psychotic symptoms comprising administering a therapeutically effective amount of one or more compounds of any one of claims 1 to 23 to a subject in need thereof.

33. A method of treating a central nervous system (CNS) disease, disorder or condition and/or a neurological disease, disorder or condition comprising administering a therapeutically effective amount of one or more compounds of any one of claims 1 to 24 to a subject in need thereof.

34. The method of claim 33, wherein the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

35. A method of treating a behavioral problem comprising administering a therapeutically effective amount of one or more compounds of any one of claims 1 to 23 to a non-human subject in need thereof.

36. The method of claim 35, wherein the non-human subject is a canine or feline suffering from neurological diseases, behavioral problems, trainability problems and/or a combination thereof.

37. The method of claim 36, wherein and the neurological diseases, behavioral problems, trainability problems include, but are not limited to, anxiety, fear and stress, sleep disturbances, cognitive dysfunction, aggression, and/or a combination thereof.

38. A method of treating a disease, disorder or condition by activation of a serotonin receptor comprising administering a therapeutically effective amount of one or more compounds of any one of claims 1 to 23 in combination with another known agent useful for treatment of a disease, disorder or condition by activation of a serotonin receptor to a subject in need thereof.

39. The method of any one of claims 28 to 38, comprising a decreased or lower risk of the subject experiencing or having serotonin syndrome.

40. A pharmaceutical composition comprising a compound of any one of claims 1 to 23 and an additional therapeutic agent.

41. The composition of claim 40, wherein the additional therapeutic agent is a psychoactive drug.

42. The composition of claim 41, wherein the additional therapeutic agent is a psychoactive drug that modifies release of serotonin or activates serotonin receptors.
